(19) United States
(12) Patent Application Publication
(10) Pub. No.: US 2017/0128059 A1
(43) Pub. Date: May 11, 2017

(54) METHODS AND APPARATUS FOR PREVENTION OF SURGICAL SITE INFECTIONS

(71) Applicant: Prescient Surgical, Inc., San Carlos, CA (US)

(72) Inventors: Jonathan Coe, Menlo Park, CA (US); Jeremy Koehler, Menlo Park, CA (US); Nicholas James Spinelli, San Carlos, CA (US); Leah Chase, Stanford, CA (US)

(21) Appl. No.: 15/344,407

(22) Filed: Nov. 4, 2016

Related U.S. Application Data
(60) Provisional application No. 62/252,313, filed on Nov. 6, 2015.

(51) Int. Cl. A61B 17/02; A61M 31/00; A61B 17/34; A61K 9/00; A61K 31/7036

(52) U.S. Cl. CPC A61B 17/0218; A61K 9/0019; A61K 31/7036; A61B 17/0293; A61B 17/3423; A61B 17/3462; A61M 31/00; A61B 2017/0225; A61B 2217/005; A61B 2217/007; A61B 2017/3488; A61B 2017/3492

(57) ABSTRACT

A surgical access device and methods for facilitating access through an incision or wound to a surgical site in a patient's body comprising an inferior retention member, a superior retention member, and a pliable membrane therebetween. The pliable membrane includes a base layer, a permeable membrane attached to the base layer, and a fluid channel disposed between the layers. The fluid channel is fluidly coupled to a fluid source. The fluid is delivered to the surgical site via the permeable membrane. The pliable membrane may also provide for fluid removal from the surgical site. The delivered fluid may comprise an antimicrobial fluid chosen to selectively inactivate or prevent the growth of a target microorganism. Methods are provided for determining the risk of surgical site infection, therapeutic regimen, target microorganism likely to require therapeutics, and/or contributing risk factors.

### CROSS-REFERENCE TO RELATED APPLICATIONS

This application claims the benefit of U.S. Provisional Application No. 62/252,313, filed on Nov. 6, 2015, entitled “Methods and Apparatus for Prevention of Surgical Site Infections” [Attorney Docket No. 43270-710.101], the entire disclosure of which is incorporated herein by reference.

This application is related to U.S. patent application Ser. No. 13/736,904, filed on Jan. 8, 2013, entitled “Expandable Tissue Retraction Devices” [Attorney Docket No. 43270-703.201]; U.S. Pat. No. 9,393,005, issued Jul. 19, 2016, entitled “Systems for the Prevention of Surgical Site Infections” [Attorney Docket No. 43270-703.202]; U.S. Pat. No. 9,084,594, issued Jul. 21, 2015, entitled “Methods for the Prevention of Surgical Site Infections” [Attorney Docket No. 43270-703.203]; U.S. Pat. No. 9,402,612, issued Aug. 2, 2016, entitled “Methods and Devices for the Prevention of Incisional Surgical Site Infections” [Attorney Docket No. 43270-704.201]; U.S. patent application Ser. No. 14/220,928, filed Mar. 20, 2014, entitled “Methods and Apparatus for Reducing the Risk of Surgical Site Infections” [Attorney Docket No. 43270-705.201]; U.S. Patent Application No. 62/325,911, filed Apr. 21, 2016, entitled “Methods and Apparatus for the Prevention of Surgical Site Infection” [Attorney Docket No. 43270-706.103]; U.S. Patent Application No. 62/332,401, filed May 5, 2016, entitled “Methods and Devices for Preventing Infections During Vascular Access” [Attorney Docket No. 43270-707.103]; U.S. patent application Ser. No. 14/739,484, filed Jun. 15, 2015, entitled “Methods for the Prevention of Surgical Site Infections” [Attorney Docket No. 43270-703.301]; U.S. patent application Ser. No. 15/186,141, filed Jun. 17, 2016, entitled “Systems for the Prevention of Surgical Site Infections” [Attorney Docket No. 43270-703.302]; and U.S. patent application Ser. No. 15/194,787, filed Jun. 28, 2016, entitled “Methods and Devices for the Prevention of Incisional Surgical Site Infections” [Attorney Docket No. 43270-704.301], the entire disclosures of which are incorporated herein by reference.

### BACKGROUND OF THE INVENTION

Formerly known as “wound infection,” surgical site infection (S SI) is generally defined by the Centers for Disease Control and Prevention (CDC) as an infection in the area of the surgical incision that occurs within 30 days of an operation. The CDC further subdivides SSI into two groups. The first group includes superficial and deep “incisional” SSI (ISSI). The second group includes “organ/space” SSI. These two groups appear to be somewhat different phenomena with respect to etiology, physiology, pathogenesis, clinical presentation, and treatment. Of note, the term “wound infection,” as currently used in the medical colloquium, refers to and is more compatible with ISSI, as opposed to organ/space SSI.

ISSI affects approximately 3-4% of the more than 30 million operations performed in the U.S. each year. Although the state of current medical care has minimized the mortality associated with ISSI, the morbidity and associated costs to the healthcare system remain significant. On average, ISSI extends the length of an inpatient hospital stay by 9 days, as well as introduces the added necessity and costs of outpatient wound management, which can reach upwards of 10,000-45,000 U.S. dollars per patient. Estimates of the aggregate annual burden to the U.S. healthcare system exceed five billion U.S. dollars.

The diagnosis of SSI is usually made by a physician and is usually based on the clinical finding of various signs and symptoms of infection at the incisional site, such as pain, tenderness, swelling, redness, warmth, and purulent drainage. Various ancillary tests, such as microbial cultures or radiographic exams (e.g., computed tomography scans), can aid in the diagnosis. The length of treatment can extend for weeks or even months.

Obese patients are particularly vulnerable to developing wound infections, with a two to three fold increased risk relative to the overall population. This is at least partially due to the poor vascularization of subcutaneous fat, reducing the delivery of prophylactic intravenous (IV) antibiotics to the incision site. Furthermore, subcutaneous fat is an excellent media for the incubation of bacterial infection. With increasing rates of obesity worldwide, this will only further compound the problem of ISSI.

Another risk factor for the development of ISSI is the type of surgical procedure performed. For example, colorectal surgeries are associated with a baseline infection rate of 15-20%. This is a result of the contaminated nature of the procedure, as fecal contents are often released into the operative field when colon, small bowel, or rectum is cut. Furthermore, colorectal surgery involves the manipulation and removal of large organs (e.g. the colon), and consequently, large incisions are often required to perform the procedures. ISSI risk is directly correlated with the size of surgical incision used to perform the case. These risks are further compounded when combined with other risk factors such as obesity. For example, the rates of wound infections in obese patients undergoing colorectal surgery increase to upwards of 33%, representing a major burden to the healthcare system in terms of the quality and cost of services.

Furthermore, the bacteria (or fungi) which cause ISSI may differ significantly on a patient-to-patient basis. In many cases, the standard treatment for ISSI may be a broad spectrum antibiotic regimen which may or may not be informed by identification of the infectious species beforehand. Treatment is also typically only given after ISSI has been diagnosed, often many days or week after surgery. Further, standard identification methods may not be sensitive enough to also identify the most effective therapeutic course of action for a given patient. It would therefore be desirable to provide for determination of the likely infectious agent(s) prior to signs of infection (e.g. prior to, during, or after surgery), determination the risk of developing ISSI, and/or determination of a therapeutic regimen to pursue.

The risk of developing a surgical site infection may be influenced by a range of factors attributable to the patient. Even organic and inorganic foreign material can result in autoimmune responses of inflammation and fibrosis. Yet ultimately, surgical site infection is caused by contamination of the surgical site itself with pathogens, a fact supported by the reality that clean operations do not yield infections, while operations having wound classifications of “clean-contaminated” to “dirty” are associated with progressively higher infection rates. Stated simply, without the invading organism, there is no infection. For example, the colon is colonized with 10⁶ to 10¹² bacteria per gram of fecal content, and when the colon is divided (to remove a diseased portion, for example), fecal content is released into the surgical site, contaminating the surgical site and exposing patients to increased infection rates. Approximately ⅔ of surgical site infections are caused by enteric bacteria, and the threshold concentration for developing infection has been shown to be approximately 10⁴ bacteria per gram, depending on host and virulence factors. In fact, studies have shown that up to 50% of abdominal wound are contaminated during surgery, and 20-33% of these contaminated wounds will go on to develop infection.

Wound protection devices that will be familiar to one skilled in the art generally consist of a first retention member, disposed within the abdominal cavity, a second retention member, disposed outside of the abdomen. A cylindrical flexible polymer sheath may be attached to the first and second retention members about its first and second openings. Changing the configuration of the second retention member, for example by rolling the second retention member about its annular axis, may be effective to shorten the sheath, retracting the surgical incision and covering the incision with a barrier that is theoretically impermeable to contaminating organisms. In a bacteriology study, wound culture swabs of the protected incision edge were found to be positive for enteric bacterial contamination with a frequency of 26% and positive for any bacterial contamination with a frequency of 34%. Although the polymer sheath may be impermeable to contaminating organisms, bacteria still manage to reach the incision surface, presumably via routes traversing the first or second retention members, cross-contamination from surgeons' gloves, or tracked into the incision when the device is removed. In addition, the design of these retractors may be such that any bacteria that do breach the barrier are subsequently protected and incubated, and can therefore multiply rapidly. Furthermore, although wound protectors were shown to reduce overall infection rates, the use of a wound protector alone was still associated with a 10% risk of surgical site infection in even some of the most recent published studies.

Therefore, it is desirable to provide for a device and method that reduces contamination of the wound with foreign material and pathogens that are known to cause infection. It would be further desirable to limit contamination of the wound with enteric bacteria, previously shown to be the most common cause of surgical site infections.

Prior surgical instruments and methods have been developed with the aim of reducing wound infections. Some solutions have addressed the issue by implanting degradable sponges in the incision to combat the development of wound infections post-operatively. However, this approach led to increases in wound infection rates, as the immune system reacts poorly to the implant because the implant is a “foreign body.”

Surgeons have previously irrigated the incision or wound margins with fluids such as saline and/or antibiotics, but the practice has proved to be disruptive to surgical progress, difficult to implement and standardize in surgical practices, and consumes valuable time, increasing patient risk and increasing operative costs. It would therefore be desirable to provide for easier application and/or removal of fluids during the surgical procedure.

Barrier wound protectors have also been employed to prevent the egress of bacteria into the incision, but this is merely a passive approach, and considering the barrier protection must be removed to complete the operation, the incision is inevitably exposed to the infectious contents contained within the surgical field. Additionally, wound protectors may be difficult to manipulate, especially when positioned in the surgical field. A further drawback is that the barrier can also trap bacteria onto the wound surface, allowing bacteria to proliferate in the wound space.

Considering the significant morbidity and cost associated with SSI, it may be desirable to provide a way to reduce the occurrence of SSI that is superior to the limitations of currently available commercial devices.

In addition to the challenges mentioned previously, in select situations, a key aspect of surgery involves obtaining adequate surgical “exposure,” or alternatively, adequate visualization and access to target anatomical landmarks and structures to be operated upon. To achieve proper exposure, surgeons can use a variety of surgical retractors generally configured to maximize the opening of the incision and create space within the operative region (e.g. chest, abdomen, orbit, neck, and groin) to facilitate the completion of the surgical procedure.

One surgical retractor used in abdominal surgery involves a top ring, bottom ring, and flexible tubular sheath disposed between the top and bottom rings. In numerous embodiments, manipulation of the top ring in a variety of ways (e.g., by rolling the sheath around the top ring) is sometimes effective to shorten the sheath length and retract the edges of the incision. In many cases, such surgical retractors incorporate barrier wound protection, the potential disadvantages of which have already been described. Furthermore, rolling sheaths may put significant tension or compression on the tissue which may not be beneficial to wound healing. It would therefore be desirable to provide a device which has reduced tissue compression compared to current devices.

The drawbacks of surgical retractors described in currently available commercial devices are numerous. They can be difficult to use, requiring additional time and the manual application of forces that may be difficult for surgeons to apply in an operative setting. They may require more than one person to operate, decreasing focus on the operative field, increasing operative time and personnel costs. In addition, due to the unpredictable nature of a surgical operation, the initial incision size may not be ideal, thus requiring lengthening during the course of the procedure. Many commercially available surgical retractors do not allow for an increase in incision size with the device in situ. Moreover, currently available commercial surgical retractors may employ a design requiring a variety of sizes to accommodate the wide range of incision sizes encountered during surgery. As a result, hospitals may have to stock a range of device sizes, and often multiple devices are used in a single procedure as the size of the incision may be increased. Using multiple devices may result in increased healthcare costs, surgery duration, and infections. It would therefore be desirable to provide a device which is easily deployable and/or adjustable.

Therefore, it would be desirable to provide improved surgical devices, systems, and methods that address SSI. Such devices and methods of use preferably are easier to use, optimize fluid management within the surgical wound, and reduce manufacturing costs and complexity. At least some of these objectives will be met by the embodiments disclosed below.

### SUMMARY OF THE INVENTION

The present invention generally relates to medical devices, systems, and methods, and more particularly relates to methods and apparatus used to facilitate access to prevent, identify the risk of, and/or treat surgical site infections.

An aspect of the present disclosure provides for a surgical method for retracting a tissue comprising: providing a surgical device comprising an expandable superior retention member, an inferior retention member, and a pliable membrane coupled therebetween; inserting the inferior retention member into a wound in a body of a patient such that the superior retention member lies above the wound; and a single user expanding the superior retention member to tension the pliable membrane, thereby retracting the wound.

Optionally, the surgical method may comprise delivering a fluid to the wound with the surgical device. The fluid may comprise a therapeutic agent.

The method may optionally comprise collapsing the superior retention member and removing the surgical device from the wound thereby allowing the wound to close.

Optionally, expanding the superior retention member may be accomplished by the single user in about 10 seconds or less.

Optionally, expanding the superior retention member can be accomplished with a maximum of two hands.

Optionally, retracting the wound may comprise applying compression to the wound by the pliable membrane without causing tissue damage.

The method may optionally comprise warming the wound and hence prevent or reduce growth of one or more microorganism. Warming the wound may comprise delivering a warm fluid to the wound. Warming the wound may comprise delivering a warm fluid to the wound with the surgical device.

The method may optionally comprise preventing desiccation of the wound. Preventing desiccation of the wound may comprise delivering a fluid to the wound. Preventing desiccation of the wound may comprise delivering a fluid to the wound with the surgical device.

The method may optionally comprise removing a fluid from the wound.

An aspect of the present disclosure provides for a surgical method for reducing a risk of a patient developing a surgical site infection comprising: pre-determining an identity of one or more microorganisms in a wound of a patient about to undergo, undergoing, or who underwent a surgical procedure; determining a therapeutic regimen that provides a treatment against the one or more microorganisms based on the pre-determined identity; and delivering the therapeutic regimen to the wound to reduce the presence of the one or more microorganisms, thereby reducing or eliminating the risk of surgical site infection.

Delivering the therapeutic regimen may be optionally accomplished using a surgical device configured for insertion into the wound.

The method may comprise removing a fluid from the wound. Removing the fluid may be accomplished using a surgical device configured for insertion into the wound.

Determining the therapeutic regimen may optionally comprise receiving one or more patient data inputs including the identity of one or more microorganisms with a processor, transforming the data with the processor, and delivering a therapeutic regimen determination to a user based on the one or more patient data inputs. The one or more patient data inputs may comprise the identity of one or more microorganisms in the wound. The one or more patient data inputs may comprise information about a microbiome of the patient.

Pre-determining the identity of the one or more microorganisms may optionally comprise receiving one or more patient data inputs with a processor, transforming the one or more data inputs with the processor, and delivering the identity of the one or more microorganisms to a user. The one or more patient data inputs may comprise the identity of one or more microorganisms in the wound. The one or more patient data inputs may comprise information about a microbiome of the patient.

An aspect of the present disclosure provides for a surgical method for retracting a tissue comprising: providing a surgical device comprising a superior retention member, an inferior retention member, and a pliable membrane coupled therebetween; inserting the inferior retention member into an wound in a body of a patient such that the superior retention member lies in a plane above the wound; and delivering an irrigation fluid or an antibiotic to the wound using the surgical device.

Delivering an antibiotic may optionally comprise delivering the antibiotic at a constant concentration.

Delivering the antibiotic may optionally comprise delivering the antibiotic without the antibiotic passing through a circulatory system of the patient prior to delivery to the wound.

Delivering the antibiotic may optionally comprise generating a concentration of the antibiotic in a tissue of the wound which is greater than a concentration of the antibiotic in a bloodstream of the patient.

Delivering the antibiotic may optionally comprise delivering the antibiotic with minimal systemic absorption of the antibiotic. Delivering the antibiotic with minimal systemic absorption of the antibiotic may reduce the risk of negative side effects to the patient. Delivering the antibiotic with minimal systemic absorption of the antibiotic may reduce a risk of acquired resistance.

Delivering the antibiotic may optionally provide at least a minimum inhibitory concentration of the antibiotic in a tissue of the wound. The minimum inhibitory concentration in the tissue of the wound may be reached within about 3 minutes of antibiotic delivery. The minimum inhibitory concentration in the tissue of the wound may be maintained for about 4 hours. The minimum inhibitory concentration in the tissue of the wound may be reached faster than by systemic delivery of the antibiotic. Systemic delivery may comprise intravenous delivery.

Optionally, the method may comprise maintaining a concentration of the antibiotic in a tissue of the wound at a constant concentration while the antibiotic is being delivered to the tissue of the wound.

Optionally, the method may comprise maintaining a concentration of the antibiotic in a tissue of the wound at a constant concentration without using intravenous delivery.

Optionally, the method may comprise maintaining a concentration of the antibiotic in a tissue of the wound within a pre-determined range.

Optionally, the method may comprise maintaining a concentration of the antibiotic in a tissue of the wound within a pre-determined range of about 16 mg/L to about 25 mg/L.

Optionally, the method may comprise maintaining a concentration of the antibiotic in a tissue of the wound within a pre-determined range without using intravenous delivery.

Optionally, the method may comprise maintaining a concentration of the antibiotic in a tissue of the wound within about 1 mg/L of a minimum inhibitory concentration of the antibiotic to a target microorganism.

Optionally, the method may comprise a concentration of the antibiotic in a tissue of the wound within a pre-determined range without intervention by a user.

Optionally, the method may comprise maintaining a concentration of the antibiotic in a tissue of the wound within a pre-determined range independent of a surgical procedure of the patient.

Optionally, the method may comprise removing the fluid from the wound. Removing a fluid from the wound may clear one or more microorganisms or debris from the wound.

Optionally, the method may comprise reducing or preventing contamination at a surface of the wound due to enteric bacteria, skin flora, gram-positive bacteria, gram-negative bacteria, aerobic bacteria, or anaerobic bacteria with the delivered antibiotic.

Optionally, the method may comprise neutralizing enteric bacteria, skin flora, gram-positive bacteria, gram-negative bacteria, aerobic bacteria, or anaerobic bacteria at a surface of the wound with the delivered antibiotic.

Optionally, the method may comprise inactivating one or more microorganisms at a surface of the wound with the delivered antibiotic.

Optionally, the method may comprise targeting one or more microorganisms at a surface of the wound with the delivered antibiotic.

Optionally, the method may comprise preventing incubation of one or more microorganisms at a surface of the wound with the delivered antibiotic.

Optionally, the method may comprise delivering the irrigation fluid or antibiotic cleanses the wound.

Optionally, the method may comprise delivering the irrigation fluid or antibiotic clears one or more microorganisms or debris from the wound.

An aspect of the present disclosure provides for a system for treating a surgical site infection comprising a processor configured with instructions and configured to (a) receive one or more patient data inputs, (b) transform the data, and (c) deliver one or more outputs that provide one or more of the following: (1) a patient risk for developing a surgical site infection; (2) an indication of a therapeutic regimen; (3) an identification of a target microorganism likely to require therapeutics; or (4) an identification of risk factors which contribute to development of surgical site infection.

The system may optionally comprise a surgical device to deliver the indicated therapeutic regimen to a tissue of a patient.

The system may optionally comprise a display coupled to the processor, wherein the display is configured to deliver the one or more outputs to a user.

An aspect of the present disclosure provides for a platform comprising a processor configured to execute instructions from one or more software modules including but not limited to (a) a sample acquisition software module comprising instructions for collecting, storing, and processing sample data associated with a subject that has undergone a surgical procedure, (b) a sample analysis software module comprising instructions for receiving and analyzing the processed sample data from the sample acquisition software module, (c) a data storage module with instructions for (i) receiving, storing, and processing the analyzed sample data from the sample analysis software module and (ii) collecting, storing, and processing assessment information, patient data, and subject data, and (d) a defense optimization and treatment module with instructions for (i) receiving the analyzed sample data, assessment information, patient data, and subject data from the data storage module, (ii) extracting a set of features from the analyzed sample data, assessment information, patient data, and subject data, the set of features reflecting factors associated with an increased risk of surgical site infection for the subject, (iii) analyzing the extracted set of features using machine learning techniques, and (iv) providing one or more recommendations for a treatment regimen decision based on the analysis of the extracted features.

Optionally, the platform may have one or more software modules comprising risk assessment software module(s) with instructions for (i) receiving the analyzed sample data, assessment information, patient data, and subject data from the data storage module, (ii) extracting a set of features from the analyzed sample data, assessment information, patient data, and subject data, the set of features reflecting factors associated with an increased risk of surgical site infection for the subject; (iii) analyzing the extracted set of features using machine learning techniques, and (iv) determining a risk associated with the subject for developing a surgical site infection based on the analysis of the extracted features. The risk assessment software module may comprise instructions for determining a most likely source of infection in the subject based on the analysis of the extracted features.

Optionally, the platform may comprise assessment information that reflects factors related to a risk of the subject developing a surgical site infection. The patient data may be associated with each one of a plurality of patients who have each undergone a surgical procedure. The subject data may reflect factors related to a risk of the subject developing a surgical site infection.

An aspect of the present disclosure provides for a computer-implemented system to provide a defense optimization and treatment application that allows a user to provide one or more recommendations for a treatment regimen decision for a subject that has undergone a surgical procedure. The system may comprise a digital processing device with (a) at least one processor, (b) an operating system configured to perform executable instructions, (c) a memory, wherein the memory comprises storage for housing sample data, assessment information, patient data, and subject data, and (d) a computer program including instructions executable by the digital processing device for (i) receiving the sample data, assessment information, patient data, and subject data, (ii) extracting a set of features from the sample data, assessment information, patient data, and subject data, the set of features reflecting factors associated with an increased risk of surgical site infection for the subject, (iii) analyzing the extracted set of features using machine learning techniques, and (iv) providing one or more recommendations for a treatment regimen decision based on the analysis of the extracted features.

Optionally, the assessment information may reflect factors related to a risk of the subject developing a surgical site infection, the patient data may be associated with each one of a plurality of patients who have each undergone a surgical procedure, and the subject data may reflect factors related to a risk of the subject developing a surgical site infection.

Optionally, the computer program of the computer-implemented system may include instructions executable by the digital processing device for at least one of determining a risk associated with the subject for developing a surgical site infection based on the analysis of the extracted features and determining a most likely source of infection in the subject based on the analysis of the extracted features.

An aspect of the present disclosure provides a non-transitory computer-readable storage media encoded with a computer program including instructions executable by a processor to provide one or more recommendations for a treatment regimen decision for a subject that has undergone a surgical procedure that comprises (a) a database, the database comprising storage for housing sample data, assessment information, patient data, and subject data and (b) a defense optimization and treatment module with instructions for (i) receiving the sample data, assessment information, patient data, and subject data from the database, (ii) extracting a set of features from the sample data, assessment information, patient data, and subject data, the set of features reflecting factors associated with an increased risk of surgical site infection for the subject, (iii) analyzing the extracted set of features using machine learning techniques, and (iv) providing one or more recommendations for a treatment regimen decision based on the analysis of the extracted features.

Optionally, the assessment information of the computer-implemented system may reflect factors related to a risk of the subject developing a surgical site infection, the patient data may be associated with each one of a plurality of patients who have each undergone a surgical procedure, and the subject data may reflect factors related to a risk of the subject developing a surgical site infection.

Optionally, the defense optimization and treatment module of the computer-implemented system may comprise instructions for at least one of determining a risk associated with the subject for developing a surgical site infection based on the analysis of the extracted features and determining a most likely source of infection in the subject based on the analysis of the extracted features.

An aspect of the present disclosure provides for a computer-implemented method for providing a treatment regimen decision for a subject who has undergone a surgical procedure comprising (a) receiving assessment information reflecting factors related to a risk of the subject developing a surgical site infection, (b) receiving patient data associated with a patient who has undergone a surgical procedure, wherein the patient data is associated with each one of a plurality of patients who have each undergone the surgical procedure, (c) receiving subject data associated with the subject, the subject data reflecting factors related to a risk of the subject developing a surgical site infection, (d) providing an automated defense optimization and treatment engine, the defense optimization and treatment engine independently performing the steps of (i) extracting a set of features from the sample data, assessment information, patient data, and subject data, the set of features reflecting factors associated with an increased risk of surgical site infection for the subject, (ii) analyzing the extracted set of features using machine learning techniques, and (iii) providing one or more recommendations for a treatment regimen decision based on the analysis of the extracted features.

Optionally, the method may comprise preprocessing the received subject data by performing one or more preprocessing operations.

Optionally, the method may comprise displaying the one or more recommendations for a treatment regimen decision on a graphical user interface.

Optionally, the assessment information of the method may comprise patient-related factors for each one of the plurality of patients, a net rank of sources of surgical site infection, and hospital-related factors. The hospital-related factors may comprise an antibiogram of the hospital.

Optionally, the patient data of the method may comprise an enteric bacterial species, a patient microbiome, and a surgical outcome.

Optionally, the subject data of the method may comprise an enteric bacterial species and a subject microbiome.

Optionally, the method may comprise factors related to a risk of the subject developing a surgical site infection that comprise at least one of the following factors: microbial density, microbial surgery, a host immune response of the subject, quality of tissue of the subject, drug resistance of the subject, virulence factors of microbial species found in the subject, a characteristic associated with the subject's wound, a systemic factor of the subject, a composition of the subject's microbiome, a surgical factor associated with the subject's surgical procedure, an anesthetic factor, and a physical condition of the subject.

Optionally, analyzing the extracted set of features may comprise at least one of determining a risk associated with the subject for developing a surgical site infection and determining a most likely source of infection in the subject. The method may comprise displaying, on a graphical user interface, at least one of the risk associated with the subject for developing a surgical site infection and the most likely source of infection in the subject.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary surgical device that may be used to prevent SSI, in accordance with embodiments;

FIG. 2A shows an expandable ring in a collapsed configuration, in accordance with embodiments;

FIG. 2B shows an expandable ring in an expanded configuration, in accordance with embodiments;

FIG. 3A shows an isometric view of the top side of the expanding linkage structure in a collapsed configuration, in accordance with embodiments;

FIG. 3B shows an isometric view of the underside of expanding linkage structure in a collapsed configuration, in accordance with embodiments;

FIG. 3C shows an isometric view of the top side of an expanding linkage structure in an expanded configuration, in accordance with embodiments;

FIG. 3D shows an isometric view of the underside of expanding linkage structure in an expanded configuration, in accordance with embodiments;

FIG. 3E illustrates a post connection feature, in accordance with embodiments;

FIG. 3F illustrates a post-receiving connection feature, in accordance with embodiments;

FIG. 3G illustrates a cross-section of an assembled pivot connecting two linkages, in accordance with embodiments;

FIG. 3H illustrates a top portion of a pliable membrane, in accordance with embodiments;

FIG. 3I illustrates an isometric view of the top side of a portion of an expanding linkage structure, in accordance with embodiments;

FIG. 3J illustrates an isometric view of the underside of a portion of an expanding linkage structure, in accordance with embodiments;

FIG. 4A illustrates an exemplary linkage structure mechanism or locking mechanism that maintains an angle between two linkages or links, in accordance with embodiments;

FIG. 4B illustrates an exemplary linkage structure mechanism in a fully collapsed configuration, in accordance with embodiments;

FIG. 4C illustrates an exemplary linkage structure mechanism in an intermediate configuration, in accordance with embodiments;

FIG. 4D illustrates an exemplary linkage structure mechanism in a fully expanded configuration, in accordance with embodiments;

FIG. 5 illustrates an exploded view of the pliable membrane, in accordance with embodiments;

FIG. 6A illustrates a top view of a surgical device comprising a locking mechanism, in accordance with embodiments;

FIG. 6B shows an expanding linkage structure in an expanded configuration, in accordance with embodiments;

FIG. 6C shows an expanding linkage structure in a collapsed configuration, wherein the device is rotated clockwise to permit collapse, in accordance with embodiments;

FIG. 7 shows a graphical representation of the results listed in Table 2, in accordance with embodiments;

FIG. 8 shows a cross-section view of an exemplary prior art barrier wound protecting surgical retractor, in accordance with embodiments;

FIG. 9 shows a cross-sectional view of a prior art surgical retractor after retracting the wound, in accordance with embodiments;

FIG. 10 shows a cross-section of a prior art surgical retractor comprising a rolling ring and a sleeve and highlighting the angle between the sleeve and the skin, in accordance with embodiments;

FIG. 11 shows a cross-sectional view of a surgical device inserted in the tissue, in accordance with embodiments;

FIG. 12 shows a cross-sectional view of a surgical device following retraction of the tissue, in accordance with embodiments;

FIG. 13 shows a cross-sectional view of a surgical device following retraction of the tissue highlighting the angle between the pliable membrane and the skin, in accordance with embodiments;

FIG. 14A shows a cross-sectional view of a surgical device configured to deliver a heated fluid to the wound tissue, in accordance with embodiments;

FIG. 14B shows an exemplary fluid delivery path comprising a heating element, in accordance with embodiments;

FIG. 14C shows an exemplary fluid delivery path comprising a plurality of switchbacks in in thermal contact with the heating element, in accordance with embodiments;

FIG. 15 shows a cross-sectional view of a surgical device comprising one or more heating elements, in accordance with embodiments;

FIG. 16A shows a cross-sectional view of a surgical device comprising fluid delivery, in accordance with embodiments;

FIG. 16B shows a pliable membrane configured to actively deliver a fluid to a wound, in accordance with embodiments;

FIG. 16C shows a pliable membrane configured to passively deliver a fluid to a wound, in accordance with embodiments;

FIG. 17A shows intraoperative delivery of a therapeutic fluid to the wound tissue, in accordance with embodiments;

FIG. 17B shows post-operative action of the therapeutic agent to prevent microbial growth, in accordance with embodiments;

FIG. 18 shows a graph of the systemic concentration of gentamicin in a healthy patient over time, in accordance with embodiments;

FIG. 19 shows a schematic of systemic antibiotic delivery compared to local fluid delivery, in accordance with embodiments;

FIG. 20 shows a schematic representation of tissue antibiotic concentration with periodic intravenous infusions in accordance with embodiments;

FIG. 21 shows a difference in antibiotic concentration between the tissue and the bloodstream when locally delivered, in accordance with embodiments;

FIG. 22 shows a surgical device inserted into a wound created in a cadaver model, in accordance with embodiments;

FIG. 23 shows a three dimensional (3D) model of a retracted surgical wound with a 12 cm incision, in accordance with embodiments;

FIG. 24 shows the model of FIG. 23 comprising a concentration gradient mesh, in accordance with embodiments;

FIG. 25 shows the results of the simulation of antibiotic concentration within the tissue at 1 mm deep over time, in accordance with embodiments;

FIG. 26 shows the results of the simulation of antibiotic concentration within bloodstream over time, in accordance with embodiments;

FIG. 27 shows a surgical device placed in a wound and expanded to open and retract the wound edges, in accordance with embodiments;

FIG. 28A shows the “exposed” swab location taken in Experiment 3, in accordance with embodiments;

FIG. 28B shows the “protected” swab location taken in Experiment 3, in accordance with embodiments;

FIG. 29A shows results of comparison testing between a surgical device with and without gentamicin delivery, in accordance with embodiments;

FIG. 29B shows serum gentamicin concentrations at Time 1 and Time 2 for the E-F treatment group of Experiment 3, in accordance with embodiments;

FIG. 29C shows tissue concentrations at Time 1 and Time 2 for the E-F treatment group of Experiment 3, in accordance with embodiments;

FIG. 30 shows a hematoxylin and eosin-stained tissue section collected from a wound in order to assess the extent of tissue damage with fluid delivery, in accordance with embodiments;

FIG. 31 shows a flowchart of a method for delivering a therapeutic agent to a surgical site using a surgical device, in accordance with embodiments;

FIG. 32 shows a flowchart of a method for identifying the bacterial species present at the surgical site in order to direct therapy, in accordance with embodiments;

FIG. 33 shows a schematic diagram of an exemplary artificial neural network which may be used to provide a desired output, in accordance with embodiments;

FIG. 34 shows exemplary matrices which may be used to structure and train the neural network, in accordance with embodiments;

FIG. 35 shows a graphical representation of the relationship between input variables and the desired output, in accordance with embodiments;

FIG. 36 shows a schematic diagram of a computer system programmed to run an artificial neural network, in accordance with embodiments;

FIG. 37 shows an exemplary input user interface, in accordance with embodiments;

FIG. 38 shows an exemplary output user interface, in accordance with embodiments; and

FIG. 39 shows a flowchart of a method of for determining a patient's risk of developing a surgical site infection to inform prophylactic treatment, in accordance with embodiments.

### DETAILED DESCRIPTION OF THE INVENTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The present invention will be described in relation to the deployment of the device or treatment of a wound during abdominal surgery. However, one of skill in the art will appreciate that this is not intended to be limiting and the devices and methods disclosed herein may be used in other anatomical areas and in other surgical procedures. Anatomical areas may for example include joint compartments, limb compartments, the thoracic cavity, the stomach, the colon, the rectum, the small intestine, the pancreas, the abdominal cavity, superficial incisions, the skin, natural body orifices, the breast, the uterus, the brain calvarium, the neck, the back or spine, or any other anatomical area known to one or ordinary skill in the art. Procedures may for example include joint replacement, arthroplasty, bone fixation, coronary artery bypass grafting, lobectomy, colorectal surgery, small intestine surgery, bariatric surgery, stomach surgery, pancreatic surgery, skin cancer removal, diabetic ulcer treatment, pressure ulcer treatment, mastectomy, hysterectomy, C-section, thyroid surgery, or other surgical procedures which may leave a wound and thus the potential for developing a surgical site as known to one of ordinary skill in the art

As noted previously, it may be advantageous to incorporate the combined functions of fluid delivery and fluid removal into a retraction device configured to reduce the risk of surgical site infections. Proposed embodiments of such a device may provide fluidic functions that are generally disposed along or near a pliable membrane, and that are configured to provide barrier wound protection (preventing direct contamination of the wound edges) and retraction of the surgical wound to permit visualization and access to the surgical site. U.S. Pat. No. 9,393,005 and U.S. patent application Ser. Nos. 15/186,141 and 13/736,904 disclose further details about such a device, the entire contents of which are incorporated herein by reference. Methods of using such a device are also disclosed in U.S. Pat. No. 9,084,594 and U.S. patent application Ser. No. 14/739,484, the entire contents of which are incorporated herein by reference. Additional disclosure about various features which may be used in such a device are disclosed in U.S. Pat. No. 9,402,612 and U.S. patent application Ser. No. 15/194,787, the entire contents of which are incorporated herein by reference. While these embodiments are preferred due to their ability to accommodate a range of incision sizes, their ability to increase the size of the incision without removing the retraction device from the surgical field, and their speed of use, among other benefits, it may be beneficial to implement fluid delivery and optionally fluid evacuation with other commercially available retractors. One such exemplary commercial retractor includes a dual ring wound retractor design described in U.S. patent application Ser. Nos. 12/873,115, and 12/119,414; U.S. Pat. Nos. 5,524,464, 7,238,154, 6,254,533, 6,814,078, 6,382,211, 8,021,296, and 8,012,088, among others. Generally, these devices are comprised of a cylindrical sheath disposed between a top and bottom ring. Shortening of the cylindrical sheath is generally effective to retract the wound opening, thereby permitting completion of a surgical procedure therethrough. It may be beneficial to combine fluid delivery and optionally fluid evacuation features with these devices to provide the advantages previously discussed above.

A preferred embodiment of a surgical device utilizes an integrated pliable membrane design that provides a barrier for wound protection and that may directly incorporate fluid delivery and removal in a single assembly.

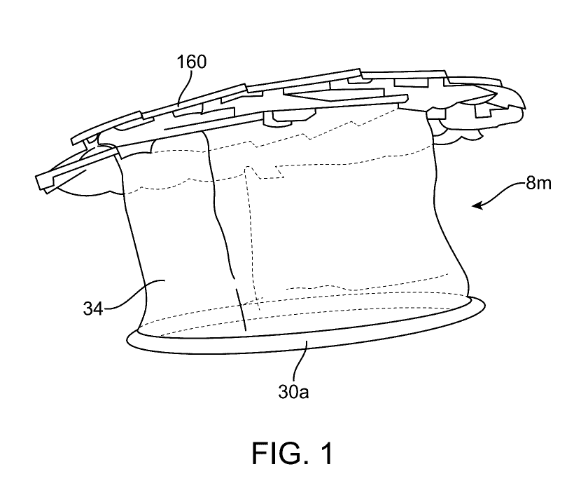

FIG. 1 shows an exemplary embodiment of a surgical device that may be used to prevent SSI. The surgical device 8m may comprise an expanding linkage structure 160 (also referred to as a retraction ring or superior retention member), a pliable membrane 34, and a retention ring 30a (also referred to as an inferior retention member). The surgical device 8m may be used to provide retraction of a surgical wound or incision 4 for surgical access as well as irrigation and suction.

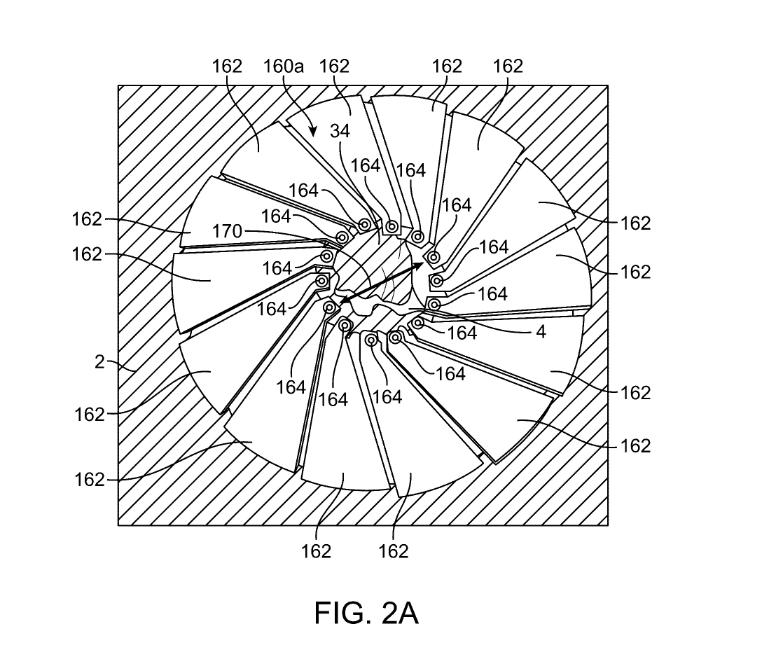

FIG. 2A shows the expandable ring 160 in a collapsed configuration 160a. FIG. 2B shows the expandable ring 160 in an expanded configuration 160b. Not all of the elements in FIG. 2B are labeled in order to make the illustration less cluttered and easier to see. For example, not all of the pivots 164, which are represented by circles, are labeled. Some of the pivots 164 are hidden by linkages 162. Not all of the linkages 162 are labeled. The patient's skin 2 in FIGS. 2A and 2B is indicated by cross hatching. Expansion of the expanding linkage 160 structure may apply tension to the pliable membrane, which may in turn engage and expand the incision as the tension structure is expanded and the tension is increased. A central channel 78 extends through the center of the pliable membrane 34 to provide access to the surgical site, which is target site 80 in FIG. 2B. The central channel 78 may remain patent (e.g. open) in the expanded configuration 160b and the collapsed configuration 160a, and in any intermediate state therebetween.

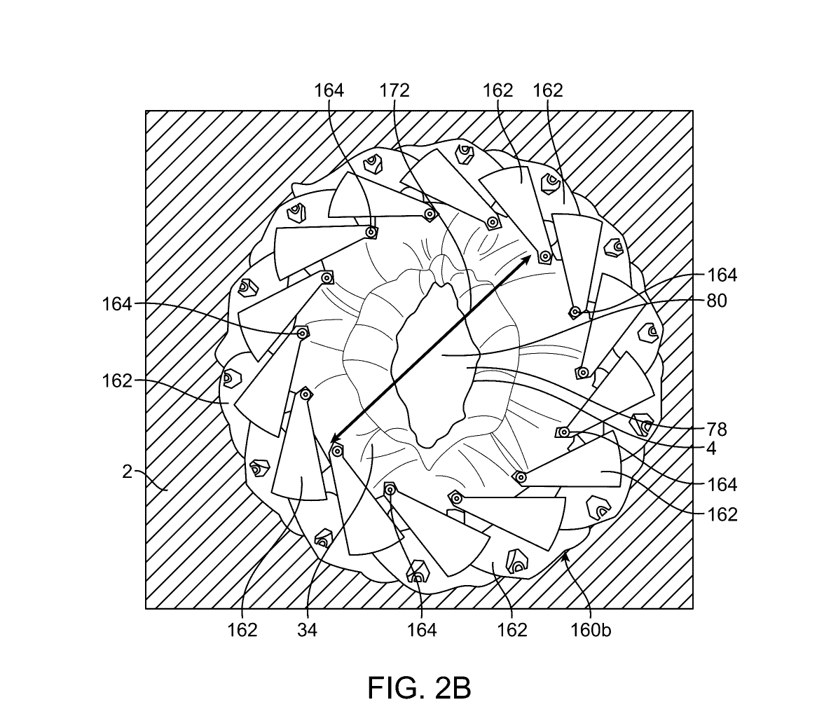

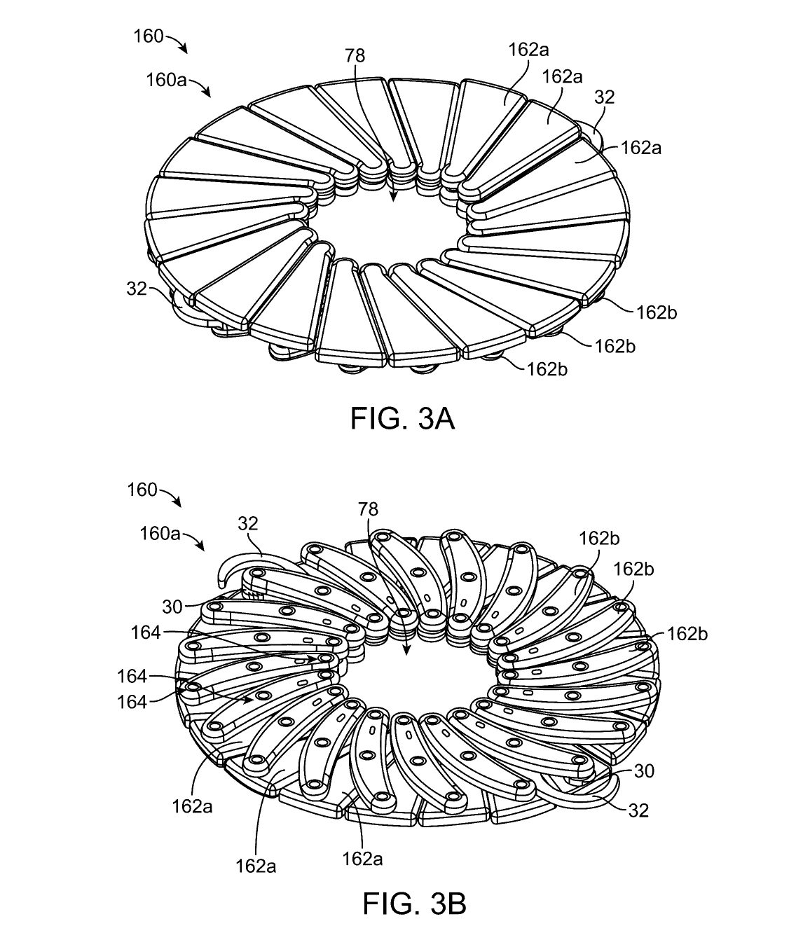

FIGS. 3A-3B illustrate an expandable ring or expanding linkage structure 160 in a collapsed configuration 160a. FIG. 3A shows an isometric view of the top side of the expanding linkage structure 160. FIG. 3B shows an isometric view of the underside of expanding linkage structure 160. The expanding linkage structure 160 may comprise a plurality of interconnected linkages 162 as in FIGS. 2A-2B. The linkages may for example comprise a plurality of top linkages 162a and a plurality of bottom linkages 162b. The long edges of the top linkages 162a may be configured to contact or engage one another such that the top side of the expanding linkage structure 160 is substantially flat when in the collapsed configuration 160a. The linkages 162a, 162b may be coupled to one another other by pivots 164. The linkages 162a, 162b may rotate about the pivots in order to radially expand or collapse the expanding linkage structure 160. The linkages 162a, 162b may be coupled to one another as shown in FIGS. 3I-4D in order to mechanically couple rotation of the top linkage 162a with rotation of the bottom linkages 162b. As shown here, each top linkage 162a is coupled to three bottom linkages 162b at pivots 164, and each bottom linkage 162b is coupled to three top linkages 162a at pivots 164 in an overlapping scissor-like pattern. Rotation of the linkages 162a, 162b about the pivots 164 may expand the expanding linkage structure 160 through circumferentially outward movement of the linkages as described herein. Actuation of the linkages 162a, 162b may cause the linkages 162a, 162b to pivot relative to one another thereby radially expanding or collapsing the expanding linkage structure 160. Actuation of the linkkages 162a, 162b may pivot radially outward in order to expand in a plane above the wound. The expanding linkage structure 160 may comprise a locking mechanism, for example a ratchet 30 and pawl 32 as described herein.

FIGS. 3C-3D illustrate an expandable ring or expanding linkage structure 160 in an expanded configuration 160a. FIG. 3C shows an isometric view of the top side of the expanding linkage structure 160. FIG. 3D shows an isometric view of the underside of expanding linkage structure 160. After expansion, the long edges of the top linkages 162a may be out of contact with one another such that the top side of the expanding linkage structure 160 comprises gaps. The expanding linkage structure 160 may comprise an inner perimeter (e.g. circumference) which defines the central channel 78 and an outer perimeter (e.g. circumference) which defines the outer edges of the expanding linkage structure 160. The inner and outer perimeters of the expanding linkage structure 160 may be circular (as shown), elliptical, triangular, rectangular, square, polygonal, or asymmetrical. The inner perimeter may be expanded from a first collapsed maximum dimension (e.g. diameter or effective diameter) or to a first expanded maximum dimension. The outer perimeter may be expanded from a second collapsed maximum dimension to a second expanded maximum dimension. The expanding linkage structure 160 may be configured such that the maximum dimensions of the inner and outer perimeters expand with a 1:1 ratio. The expanding linkage structure 160 may be configured such that the maximum dimensions of the inner and outer perimeters expand with a ratio greater than 1:1. The expanding linkage structure 160 may be configured such that the maximum dimensions of the inner and outer perimeters expand with a ratio less than 1:1. The linkages may be pivotably connected as shown. Alternatively or in combination, one or more of the linkages may be slideably connected.

FIGS. 3E-3G illustrate a pivot assembly 164 between a top linkage 162a and a bottom linkage 162b. The top linkage 162a may comprise a post 164a which is configured to be inserted into a hole 164b disposed in the bottom linkage 162b. The post 164a and hole 164b may be configured to couple together with a snap-fit such that the post 164a may not be disengaged from the hole 164b. Alternatively, the post 164a and hole 164b may be configured to removably coupled. The post 164a and hole 164b are just one possible mechanism for generating a pivot 164. It will be understood by one of ordinary skill in the art that any number of mechanisms may be used to pivotably couple the top linkage 164a and bottom linkage 164b.

FIG. 3H illustrates a top portion of a pliable membrane. The pliable membrane 24 may comprise a plurality of holes 45 near the superior (upper) perimeter 36 of the pliable membrane 34. The holes 45 may be configured to engage innermost pivots 164 to couple the pliable membrane 34 to the expanding linkage structure 160. The post 164a may for example be sized and shaped to fit within the hole 45. The bottom linkage 162b may then be coupled to the post 164a of the top linkage 162a by the hole 164a in order to create the pivot around the hole 45 of the pliable membrane. The perimeter 46 of the pliable membrane 34 may be scalloped (as shown), elliptical, triangular, rectangular, square, polygonal, or asymmetrical as desired or known to one of ordinary skill in the art. The pliable membrane 34 may be coupled to the expanding linkage structure 160 so as to avoid having or reduce the amount of pliable membrane 34 between the linkages which may impair movement of the expanding linkage structure 160. Attachment of the pliable membrane 34 at or near the inner perimeter of the expanding linkage structure 160 may apply symmetric (e.g. uniform) or near symmetric tension to the pliable membrane 34. Uniform tension along the pliable membrane 34 may allow the pliable membrane 34 to symmetrically radially expand as the expanding linkage structure 160 is symmetrically radially expanded. Symmetric radial expansion of the pliable membrane 34 may provide for uniform expansion of the wound.

FIG. 3I illustrates an isometric view of the top side of a portion of an expanding linkage structure 160 coupled a pliable membrane 34. FIG. 3J illustrates an isometric view of the underside of a portion of an expanding linkage structure 160 coupled to a pliable membrane 34. The pliable membrane 34 may be coupled to the expanding linkage structure 160 at the innermost pivots 164 as described herein. The pliable membrane 34 may comprise holes (not shown) which may be disposed about the innermost posts 164a of the top linkages 162a (along the inner perimeter of the expanding linkage structure 160). The bottom linkages 162b may comprise innermost holes 164b which may be coupled to the innermost posts 164a to form the pivots 164. The inner most pivots 164 may comprise a portion of the pliable membrane 34.

Not all of the elements in FIGS. 3A-3J are labeled in order to make the illustration less cluttered and easier to see. For example, not all of the pivots 164, which are represented by circles, are labeled. Some of the pivots 164 are hidden by linkages 162a, 162b. Not all of the linkages 162a, 162b are labeled. Not all of the posts 164a or holes 164b which make sure the pivots 164 are labeled or shown.

Additional details about the surgical device and how it may be used are disclosed in U.S. Pat. Nos. 9,393,005 and 9,084,594 and U.S. patent application Ser. Nos. 13/736,904; 15/186,141; and Ser. No. 14/739,484; the entire contents of which are incorporated herein by reference.

FIG. 4A illustrates an exemplary embodiment of a linkage structure mechanism or locking mechanism that maintains an angle 40 between two linkages or links in the expandable ring described above. A ratchet/pawl mechanism may be used with a ratchet 30 disposed about a post 38 on a first link 31 and a pawl 32 disposed about a post 39 on a second link 33. Third link 44 and fourth link 35 constrain the first 31 and the second 33 links to rotate in accordance with the full expanding structure. The ratchet 30 could be rotationally constrained by a post 38. A pawl tooth 36 may engage a tooth 37 on the ratchet 30. This engagement could prevent the links from rotating in a direction such that the angle 40 formed between lines connecting an outermost post 38 and middle post 41 of a first link 31 and an outermost hole 42 and a middle hole 43 of a third link 44 decreases. This decrease in angle 40 would be required for the effective inner diameter of the structure to decrease in order to collapse the structure as described herein. This mechanism would therefore selectively maintain an intermediate state.

FIG. 4B illustrates an exemplary linkage structure mechanism 160 in a fully collapsed configuration 160a. FIG. 4C illustrates an exemplary linkage structure mechanism 160 in an intermediate configuration 160c. FIG. 4D illustrates an exemplary linkage structure mechanism 160 in a fully expanded configuration 160b. As the expanding linkage structure 160 is expanded, the pawl tooth 36 may engage a plurality of teeth on the ratchet 30 to prevent the links from collapsing. In the collapsed configuration 160a, the pawl tooth 36 may engage a first tooth 37a on the ratchet 30. In an intermediate configuration 160c, rotation of the links may partially expand the expanding linkage structure 160 and the pawl tooth 36 may engage a second tooth 37b on the ratchet 30. In the expanded configuration 160b, rotation of the links may fully expand the expanding linkage structure 160 and the pawl tooth 36 may engage a third tooth 37c. It will be understood by one of ordinary skill in the art that the ratchet may comprise any number of teeth desired in order to secure the expanding linkage structure 160 in any number of intermediate configurations 160c.

Additional details about the expanding linkage structure mechanisms and other locking mechanisms are disclosed in U.S. Pat. Nos. 9,402,612, 9,393,005, and 9,084,594 and U.S. patent application Ser. Nos. 15/194,787; 13/736,904; 15/186,141; and Ser. No. 14/739,484; the entire contents of which are incorporated herein by reference.

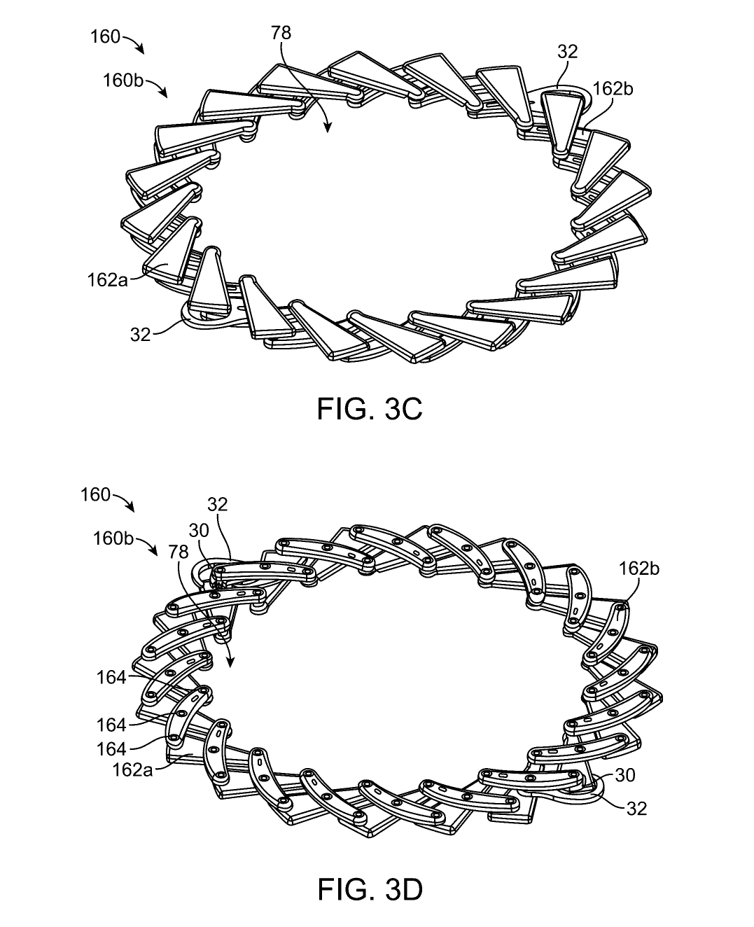

FIG. 5 illustrates an exploded view of a preferred embodiment of the pliable membrane 34. The pliable membrane 34 may comprise several layers of material laminated together. The pliable membrane 34 may include a base layer such as impermeable layer 21, a foam manifold 22, a foam manifold seal layer 23, and/or a semi-permeable layer 25. Suction windows 24 may be disposed in the semi-permeable layer 25 and/or the foam manifold seal layer 23. Assembly of the layers may form an integrated pliable membrane design. The pliable membrane 34 may be configured to provide for fluid delivery to and fluid removal from the wound space or surgical site. Fluid delivery and/or fluid removal may be provided by one or more perforations in the pliable membrane and one or more channels or spaces defined between the semi-permeable layer 25 and any of the other layers of the pliable membrane. The semi-permeable layer 25 may have perforations defining fluid exit locations to permit fluid delivery to the wound, delivered via connection to an externally pressured fluid source. Alternatively or in combination, the semi-permeable layer 25 may have perforations defining fluid egress locations to permit fluid removal from the wound, removed via connection to an external vacuum source. Alternatively or in combination, fluidicly separate channels (or chambers) may be defined by the semi-permeable layer and configured to provide a plurality of locations for fluid delivery and/or removal. Additional details about the pliable membrane and how it may be manufactured and used for fluid delivery, fluid removal, and wound retraction are disclosed in U.S. Pat. No. 9,402,612 and U.S. patent application Ser. No. 15/194,787; the entire contents of which are incorporated herein by reference.

The surgical device or any of its components may have any of the features described herein or in the following applications: U.S. Pat. Nos. 9,393,005, 9,084,594, 9,402,612 and U.S. patent application Ser. Nos. 13/736,904, 14/220,928, 62/325911, 62/332,401, 14/739,484, 15/186,141, and 15,194,787; the entire contents of which are incorporated herein by reference, in any combination of features.

Surgical Device Ease of Use

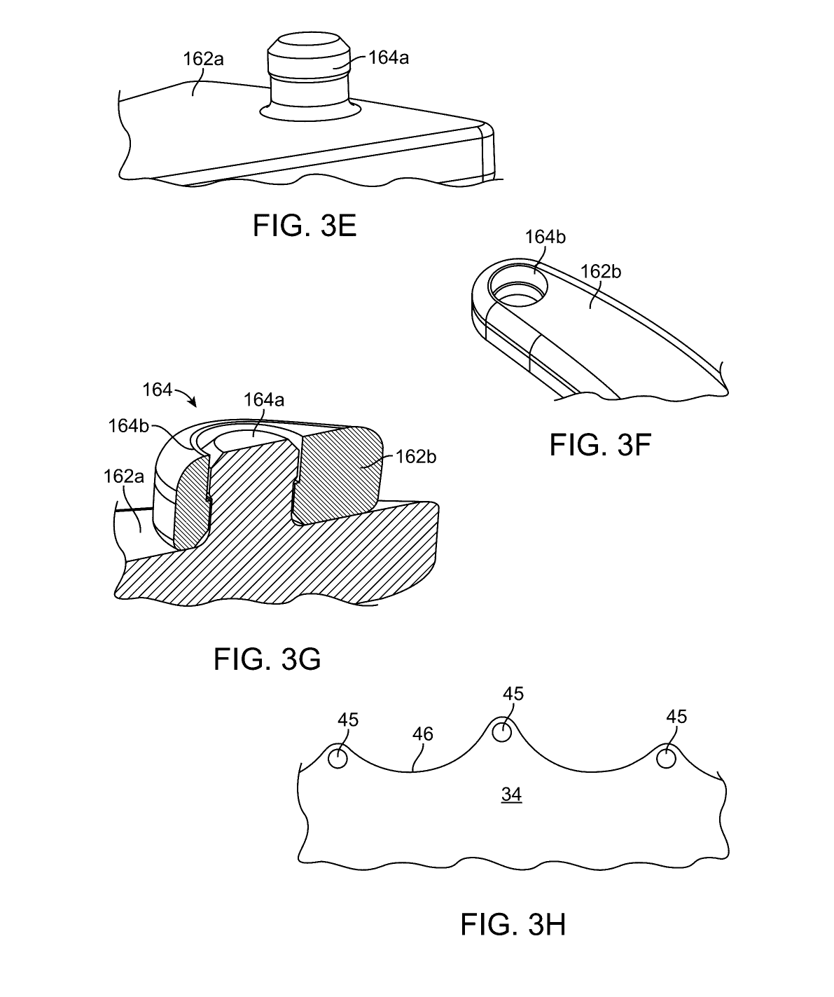

FIGS. 6A-6C illustrate a top view of a surgical device in use. The surgical device 8m may comprise an expanding linkage structure 160, a pliable membrane 34, and a retention ring (not shown) as described herein. The surgical device 8m may further comprise a locking mechanism 610 as shown in FIG. 6A, for example the ratchet/pawl mechanism described in FIG. 4. The surgical device 8m may further comprise fluid delivery and/or fluid removal as described herein (not shown). FIG. 6B shows the expanding linkage structure 160 in an expanded configuration 160b. FIG. 6C shows the expanding linkage structure in a collapsed configuration 160a. A user 600 may release the locking mechanism 610 to adjust the expanding linkage structure 160 form the expanded configuration 160b to the collapsed configuration 160a. The expanding linkage structure 160 may be collapsed radially inward so as to reduce tension on the pliable membrane 34 and allow the surgical device 8m to be removed or adjusted. Alternatively or in combination, rotation clockwise or counterclockwise of the expanding linkage structure 160 after release of the locking mechanism 610 as shown may affect release of wound retraction. Rotation of the expanding linkage structure 160 after release of the locking mechanism 610 may generate tension in the pliable membrane 34 which is directed radially inward. The inward tension in the pliable membrane 34 may collapse the central channel 78 of the surgical device 8m as shown in FIGS. 6B-6C. Rotation and/or radially collapse of the expanding linkage structure 160 may provide a smooth closure motion which may reduce splashing of bodily fluids thereby reducing contamination of the user 600.

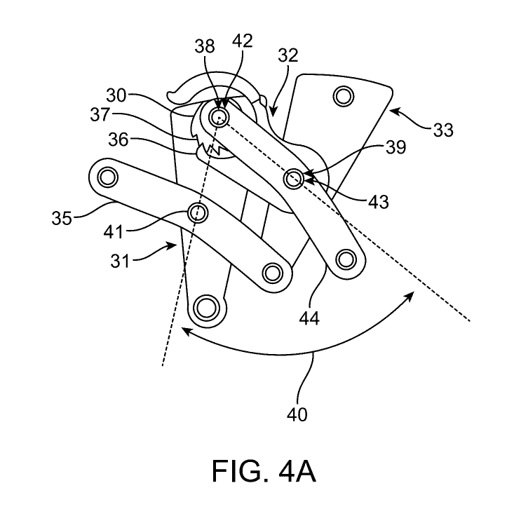

In many instances, it may be beneficial to provide a surgical device 8m which may be operated by a single user. Other wound protection devices, for example as in FIGS. 8-10, often require two or more people to operate as tension is provided by evenly rolling the pliable membrane 12 around the rolling ring 18. The surgical device 8m may be configured such that the user 600 may operate the device with one hand or both hands. Operation of the surgical device 8m may include expanding the device 8m, collapsing the device 8m, irrigating the wound, removing fluid, or any combination thereof.

The surgical device 8m may be expanded or collapsed more quickly than previous devices due to the ease of use as described herein. The surgical device may be expanded or collapsed in a single, contiguous motion. The surgical device 8m may be expanded (or collapsed) in less than about 10 seconds, for example less than about 5 seconds, for example less than about 1 second. For example, the surgical device 8m may be expanded (or collapsed) within a range of about 0.5 second to about 10 seconds, within a range of about 1 to about 5 seconds, within a range of about 0.5 to about 3 seconds, or about 1 second.

In many cases, the central channel running through the pliable membrane and providing access to the surgical site may remain open for the duration of the surgical procedure. In some embodiments, it may be beneficial to seal the wound and prevent access to the surgical site during surgery without removing the surgical device 8m from the wound, for example to temporarily seal the site against gas and/or liquids. During some laparoscopic surgeries, for example, it may be beneficial at times to access the surgical site through multiple openings. The surgical site may be accessed via a larger opening as provided by the central channel of the surgical device 8m. The larger opening may then be sealed so as to allow the surgical site, for example the abdomen, to be inflated with a fluid such as carbon dioxide in order to continue the surgery using laparoscopic techniques. By configuring the surgical device 8m to seal the wound and close the central channel while remaining in the surgical site, the surgical device 8m may reduce the amount of time required to complete a surgery as the surgeon or other healthcare provider may continue the surgery without removing the retractor and surgically sealing the wound. The number of surgical personnel required to complete a surgery may be reduced as the surgical device 8m may be easily and quickly sealed by the user 600 without the need for additional hands to help remove the device 8m and/or surgically seal the wound. The surgical device 8m may be configured such that rotation of the expanding linkage structure 160 without release of the locking mechanism 610 causes the pliable membrane 34 to twist between the expanding linkage structure 160 and the retention ring to effectively seal the central channel 78.

### Experiment 1

A prototype of the surgical device 8m was inserted into an abdominal surgical incisional wound in a human cadaver in order to closely mimic actual surgical conditions. Five board-certified surgeons, both general and colorectal surgeons, were asked to compare the surgical device to the commercially-available Alexis® Wound Protector device for clinical usability and functionality throughout the entirety of its intended use (e.g. from placement in the wound and expansion to removal from the wound). The surgical device provided wound retraction, barrier wound protection, circumferential fluid delivery to the wound margins, and circumferential fluid removal from the wound margins as described herein. The Alexis® Wound Protector only provided wound retraction and barrier wound protection, thus the fluid delivery and fluid removal features were not scored in comparison to the Alexis®.

Table 1 lists the questions asked of surgeon-users with respect to the usability and functionality of the surgical device compared to the commercially-available Alexis® Wound Protector device.

TABLE 1
Usability and Functionality Questions

| Question | |
|---|---|
| number | Question text |
| 1 | On a scale of 1 to 5, 1 being very poor and |
| | 5 being excellent, please rate your level of |
| | understanding of how to use the device. |
| 2 | On a scale of 1 to 5, 1 being very difficult |
| | and 5 being very easy, please rate how easy or |
| | difficult it was it for you to place the device |
| | in the wound. |
| 3.1 | On a scale of 1 to 5, 1 being very poor and 5 |
| | being excellent, please rate how well the device |
| | (by itself) retracts and expands the wound, to |
| | allow adequate visualization and exposure. |
| 3.2 | On a scale of 1 to 5, 1 being very poor and 5 |
| | being excellent, please rate how compatible you |
| | believe this device to be with other body habitus |
| | types as well as other incision sizes and |
| | characteristics. |
| 3.3 | On a scale of 1 to 5, 1 being very poor and 5 |
| | being excellent, please rate how well you believe |
| | this device functions to protect the wound margins |
| | from direct contamination at this point in the |
| | procedure. |
| 3.4 | On a scale of 1 to 5, 1 being very traumatic and 5 |
| | being atraumatic, please rate the level of tissue |
| | injury you believe the device is exerting on the |
| | wound margins at this point in the procedure. |
| 3.5 | On a scale of 1 to 5, 1 being very disruptive and 5 |
| | being very convenient, please rate how easy or |
| | difficult it was for you to set up and activate |
| | the fluid mechanism. |
| 3.6 | On a scale of 1 to 5, 1 being very poor and 5 being |
| | excellent, please rate how effectively you believe |
| | the fluid mechanism delivers fluid to and from the |
| | wound intraoperatively. |
| 3.7 | On a scale of 1 to 5, 1 being very poor and 5 being |
| | excellent, please rate how effectively the device |
| | minimizes fluid leakage and/or fluid collection |
| | deeper in the operative field. |
| 4 | On a scale of 1 to 5, 1 being very difficult and 5 |
| | being very easy, please rate how easy or difficult |
| | it was to extend the incision, with the device in |
| | place. |
| 5.1 | On a scale of 1 to 5, 1 being very poor and 5 being |
| | excellent, please rate how well you were able to |
| | achieve the exposure you desired with the aid of |
| | the handheld retractors. |
| 5.2 | On a scale of 1 to 5, 1 being very poor and 5 being |
| | excellent, please rate the compatibility of this |
| | device with the use of the handheld retractors. |
| 6.1 | On a scale of 1 to 5, 1 being very poor and 5 being |
| | excellent, please rate how well you were able to |
| | achieve the exposure you desired with the aid of |
| | the self-retaining retractors. |
| 6.2 | On a scale of 1 to 5, 1 being very poor and 5 being |
| | excellent, please rate the compatibility of this |
| | device with the use of the self-retaining retractors. |
| 7.1 | On a scale of 1 to 5, 1 being very poor and 5 being |
| | excellent, please rate how well you believe this |
| | device functions to protect the wound margins from |
| | direct contamination at this point in the procedure. |
| 7.2 | On a scale of 1 to 5, 1 being very difficult and 5 |
| | being very easy, please rate how easy or difficult |
| | it was to work with the exteriorized bowel with the |
| | device in place. |
| 7.3 | On a scale of 1 to 5, 1 being very disruptive and 5 |
| | being very convenient, please rate how easy or |
| | difficult it was for you to stop the fluid mechanism. |
| 7.4 | On a scale of 1 to 5, 1 being very poor and 5 being |
| | excellent, please rate how effectively you believe |
| | the fluid mechanism delivered fluid to and from the |
| | wound, throughout the procedure. |
| 7.5 | On a scale of 1 to 5, 1 being very poor and 5 being |
| | excellent, please rate how effectively the device |
| | minimized fluid leakage and/or fluid collection |
| | deeper in the operative field, throughout the |
| | procedure. |
| 8 | On a scale of 1 to 5, 1 being very disruptive and 5 |
| | being very convenient, please rate how easy or |
| | difficult it was for you to remove the device. |
| 9 | Please rate the overall effectiveness and ability of |
| | the device in meeting your objectives and |
| | requirements for this type of surgical procedure |
| | (good, adequate, or difficult). |

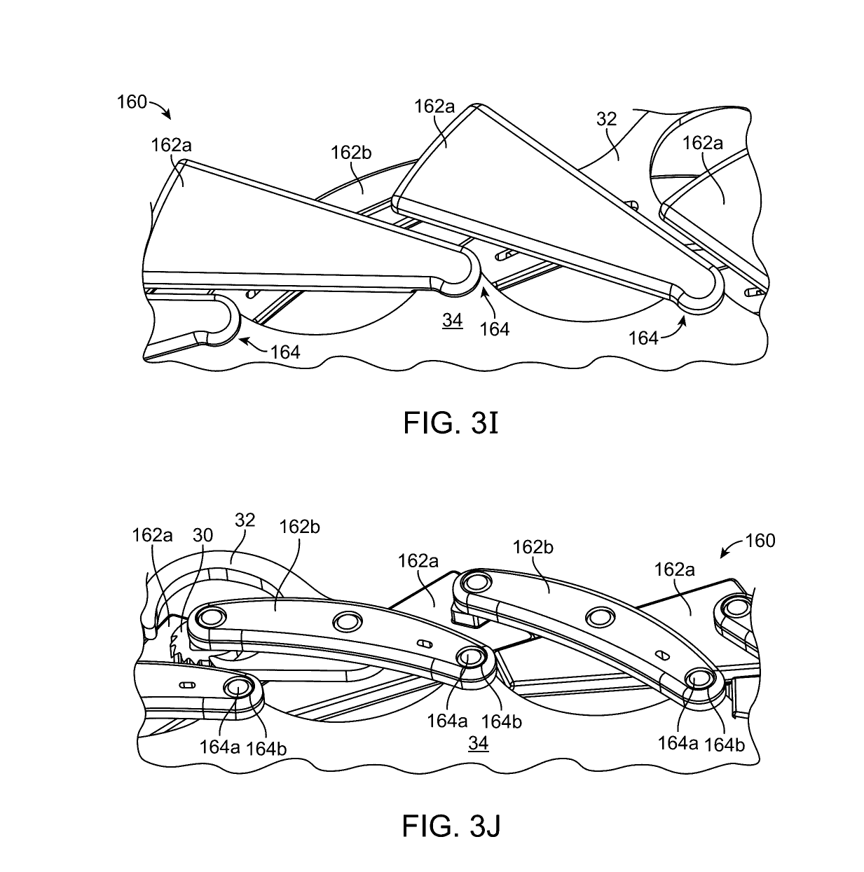

Table 2 shows a comparison of mean scores (plus or minus standard deviation (SD)) of the surgical device and Alexis® for each usability question asked of the test surgeons. The surgeons were asked to answer each of Questions 1-8 on a scale of 1 to 5, where 1 was the least usable (most difficult), 3 was acceptable (adequate), and 5 was the most usable (easiest). FIG. 7 shows a graphical representation of the results listed in Table 2 with the results in descending order of mean score difference between the surgical device and the Alexis®. Question 9 was rated on a scale from 1 to 3, with 1 being difficult overall, 2 being adequate overall, and 3 being good overall.

TABLE 2
Means Scores for Usability and Functionality Questions.

| Question | Mean score (+/−SD) | Mean score (+/−SD) for |
|---|---|---|
| number | for the Alexis ® | the surgical device |
| 1 | 5 ± 0 | 5 ± 0 |
| 2 | 4.8 ± 0.4 | 4.9 ± 0.2 |
| 3.1 | 4.4 ± 0.5 | 4.9 ± 0.2 |
| 3.2 | 3.6 ± 0.9 | 4.9 ± 0.2 |
| 3.3 | 5 ± 0 | 5 ± 0 |
| 3.4 | 4.2 ± 8 | 5 ± 0 |
| 4 | 3.8 ± 1.1 | 4.8 ± 0.4 |
| 5.1 | 4.5 ± 0.7 | 4.8 ± 0.3 |
| 5.2 | 5 ± 0 | 5 ± 0 |
| 6.1 | 4.6 ± 0.5 | 5 ± 0 |
| 6.2 | 4.8 ± 0.4 | 5 ± 0 |
| 7.1 | 5 ± 0 | 4.8 ± 0.4 |
| 7.2 | 4.8 ± 0.4 | 5 ± 0 |
| 8 | 5 ± 0 | 4.8 ± 0.4 |

The performance of the Alexis® for each of the individual user tasks was generally acceptable (e.g. received a rating of 3 or higher). Only 1 out of 5 surgeons gave an “unacceptable” rating (e.g. less than 3) to any of the questions (Question 4 regarding extension of the incision length with the device in place). On a three-point scale for the final summary question on the overall effectiveness of the device (Question 9), the Alexis® was rated a mean score of 2.8+/−0.4. The most prevalent qualitative comments about the Alexis® were that 1) the Alexis® was a familiar device, 2) rolling the outer ring of the Alexis® was quite difficult, 3) the Alexis® was an effective wound retractor if the incision size strictly matched the retractor size, and 3) the Alexis® created both usability and hospital inventory problems when the incision was lengthened past the diameter of that particular size model's non-adjustable outer ring.

The performance of the surgical devices for the same user tasks as those performed with the Alexis® was uniformly acceptable, with no “unacceptable” ratings on any of the questions. Specifically for the deployment function, surgeons found that the force required to deploy (e.g. expand) the surgical device, which was less than 10N for the prototypes used in this study, was acceptable. The surgical device performed at a level statistically equivalent to the Alexis® in all of the overlapping functions, and for two questions (Questions 3.2 and 4, which related to the fluid delivery and fluid removal functions which only the surgical device had) the surgical device scored higher than the Alexis® and approached statistical significance. Increasing the power of the study (e.g. by adding additional test surgeons) may make the differences between the usability and the surgical device and the Alexis® clearer. On the three-point summary effectiveness question (Question 9), the surgical device was given a unanimous perfect score of 3, compared to the score of 2.8+/−0.4 given to the Alexis®. Common comments included the following: 1) the actuation mechanism for wound retraction was easier in the surgical device than that of the Alexis®, with an acceptable force required for deployment, 2) the barrier sheath (e.g. pliable membrane) coverage offered a larger surface than the Alexis® to protect from contamination, 3) the surgical device device was more versatile for a wider variety of body habitus and incision lengths, and 4) the prototype surgical device was acceptable for clinical testing.

This human cadaver-based clinical usability study of the latest surgical device prototype demonstrated an overall high level of effectiveness in satisfying the Customer Requirement Specifications for this product. The surgical device performed at an equivalent or superior level compared to the Alexis® device in satisfying the Customer Requirements related to the surgical device's function as a barrier wound protector and retractor. Surgeons found that the force required to deploy the surgical device, which was less than 10N for the prototypes used in this study, was acceptable. In addition, the effectiveness of the fluid delivery and retrieval mechanism, which are unique to the surgical device, was rated at a uniformly high level by the surgeon-users. The feedback indicated that the device could prevent a majority of fluid from leaking into the abdominal cavity. In summary, the technical usability of the current surgical device prototype build has been deemed acceptable for clinical testing in human subjects.

Reduction of Wound Compression

The surgical device 8m may be configured to provide reduced tissue compression during tissue retraction compared to other surgical retractors or barrier wound protectors. The surgical device 8m may be configured to reduce compression and provide lower compressive forces to the tissue due to the geometry of the device, the low profile of the device, the direction of motion of retraction, and/or the angle between the pliable membrane 34 and the skin. Lower tissue compression may lead to a lower likelihood of pressure-induced necrosis of the skin and wound tissue (e.g. abdominal tissues in the case of abdominal surgery).

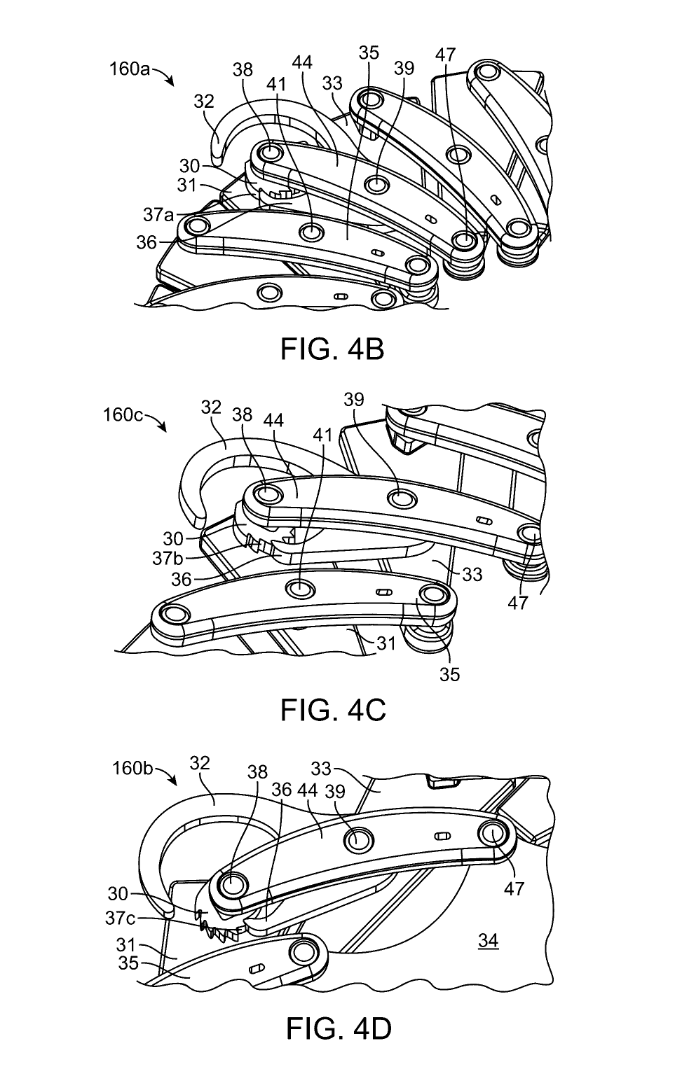

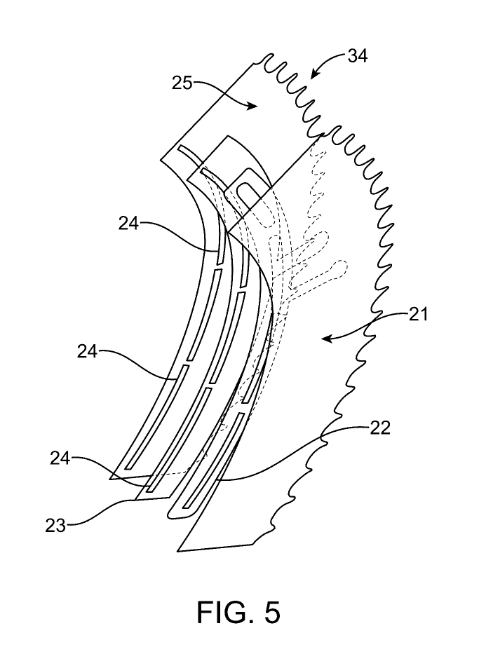

FIGS. 8-10 show an exemplary prior art barrier wound protecting surgical retractor. FIGS. 8-9 are cross-sectional views of a prior art surgical retractor which have been reproduced from U.S. Pat. No. 5,524,644 for illustration purposes. As shown, the surgical retractor comprises a sleeve 12, a rolling ring 18, and an inner ring 20. The outer end portion 12a of the sleeve 12 wraps around the rolling O-ring 18 and extends through the wound W₁ to the inner ring 20. The outer end portion 12a may be turned radially outwardly, as shown by arrows A, to roll the sleeve 12 about the rolling ring 18 from an initial position as shown in FIG. 8 until the sleeve 12 contacts the tissue and the ring 18 reaches skin level as shown in FIG. 9. Additional rolling of the sleeve 12 around the ring serves to tension the sleeve 12 and retract the wound W₁. FIG. 10 shows a cross-section of the surgical retractor comprising the rolling ring 18 and the sleeve 12. Tension in the sleeve 12 may be comprised of two component vectors—a component normal to the wound surface (e.g. aligned with the x-axis) and a component normal to the skin (e.g. aligned with the y-axis). The component normal to the wound surface may serve to retract the wound. The component normal to the skin may be detrimental, for example by compressing the wound tissue which may lead to pressure necrosis and/or tissue damage. The compression force may be defined by the amount of tension in the sleeve 12 and the angle θ₁ between the sleeve 12 and the tissue according to the Equation 1.

F_{compression}=T·sin θ₁ (1)

Given the fixed diameter of the rolling ring 18, retraction of the wound W₁ increases the angle θ₁ between the sleeve 12 and the rolling ring 18, thereby increasing the portion of the tension that compresses the tissue. Additionally, as the width of the incision approaches the diameter of the rolling ring 18, the majority or all of the tension could be applied as compressive force on the tissue (θ₁→90°).

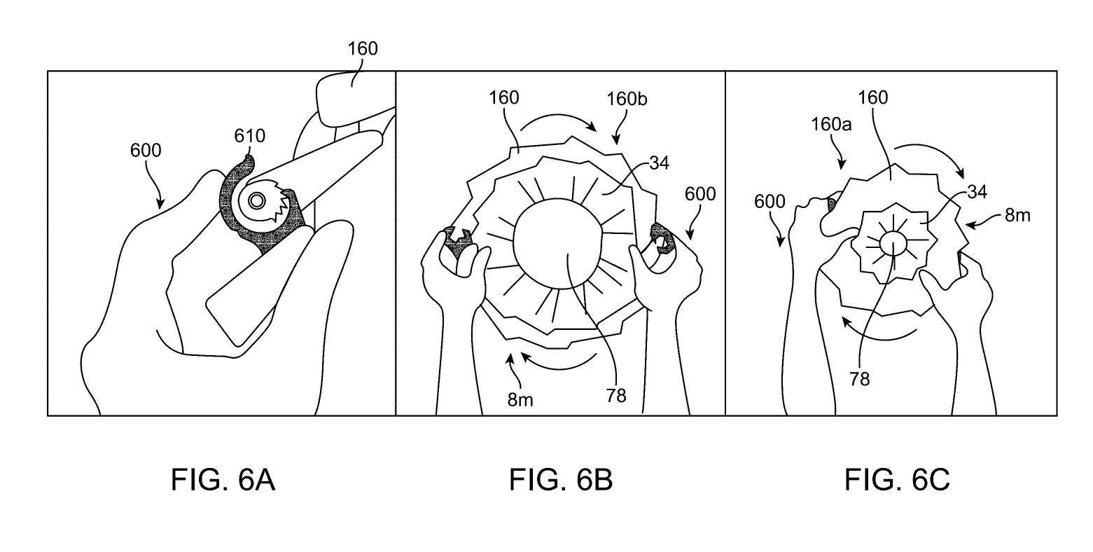

FIGS. 11-13 show cross-sectional views of the surgical device 8m being expanded to retract a surgical wound or incision 4. Radial or outward expansion of the expanding linkage structure 160 may apply tension to the pliable membrane 34 which may in turn apply tension to the wound 4 to expand the wound 4 in the direction indicated by the arrows. Unlike the surgical retractor shown in FIGS. 8-10, the surgical device 8m may be inserted and tension begun with the expanding linkage structure 160 is above the level of the skin 2 as shown in FIG. 11. A component of the tension applied against the tissue may be in a compressive direction. The compression force may be defined by the amount of tension in the pliable membrane 34 and the angle θ₁ between the pliable membrane 34 and the tissue 4 according to the Equation 2.

F_{compression}=T·sin θ₂ (2)

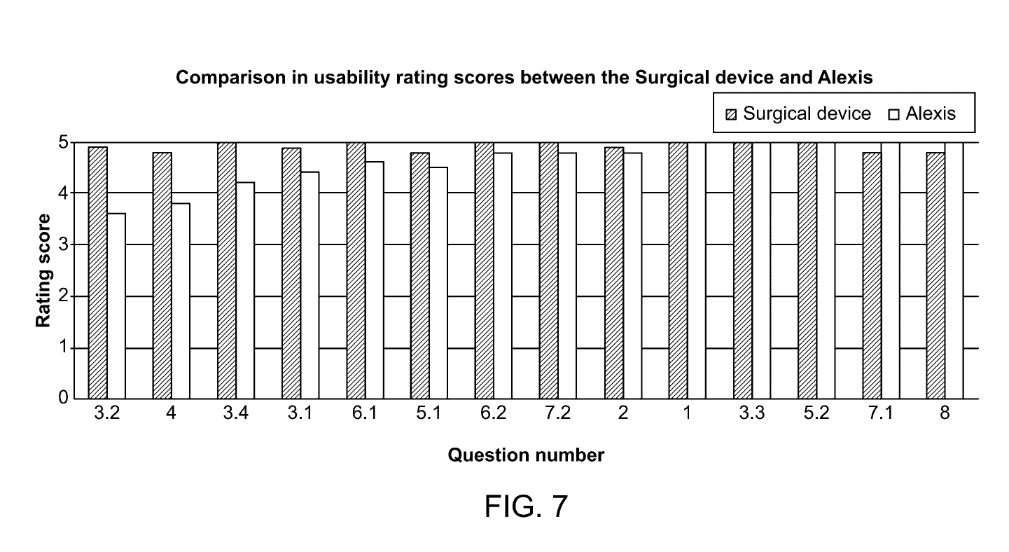

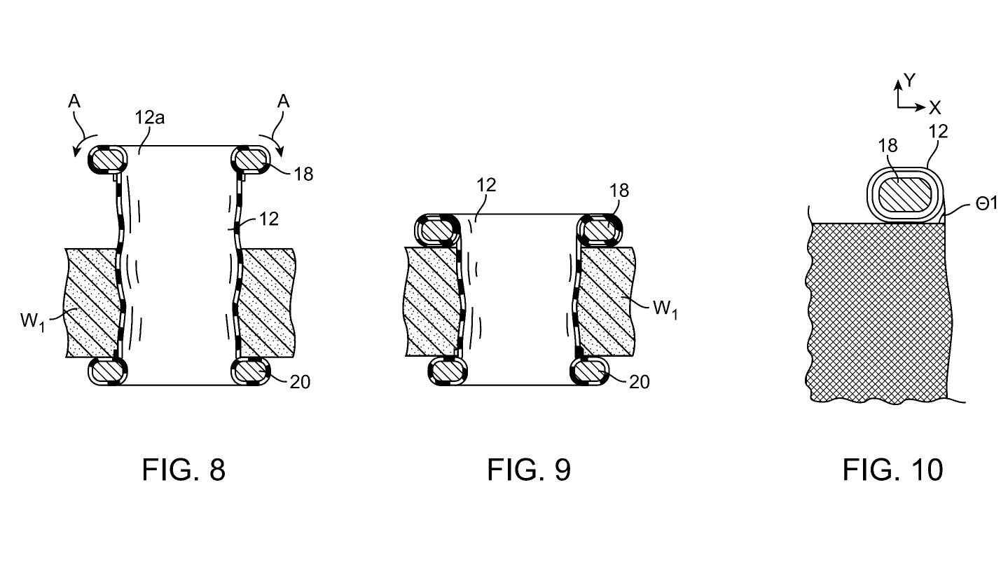

As the expanding linkage structure 160 is radially expanded, the angle θ₂ between the pliable membrane 34 and the tissue 4 may be reduced, thereby reducing the amount of compressive force applied against the wound tissue 4 as the expanding linkage structure expands and/or gets closer to the skin 2 as shown in FIGS. 12-13. For a given amount of tension necessary to retract a wound, the surgical device 8m may thus impart a lower amount of compressive force on the tissue compared to existing surgical retractors. The surgical device 8m may be configured to apply compression to the wound without causing tissue damage

The pliable membrane may be configured to apply a pre-determined amount of tension to the tissue in order to retract the wound. The pliable membrane may be configured to apply a force to the tissue within a range of about 10N to about 100N, for example about 50N. The pliable membrane may be configured to apply pressure to the tissue of less than about 30 mmHg, for example about 8 mmHg or less. It will be understood by one of ordinary skill in the art that the pliable membrane may be configured to apply any tension, force, or pressure to the tissue as desired.

Temperature Control

In many cases, it may be beneficial to maintain the temperature of the patient (e.g. normothermia) during surgery. Mild hypothermia can cause increased blood loss, prolonged post-anesthesia recovery, prolonged hospitalization, an increase in morbid myocardial events, increased wound infection, and/or longer healing duration. For example, during open abdominal surgery, the abdominal wall may facilitate access to the surgical site of interest. The open body cavity may create a large opening through which the body may lose heat. The blood vessels may be constricted and the internal organs may be exposed to the external environment. Normothermia can be maintained several ways including the use of blankets, hot pads, hot water fluid circulation devices, and other heating apparatuses. Using devices, drapes, blankets, etc. may provide heat to much of the body's surface are, however the surgical site may remain exposed to the environment in order for the surgery to be performed.

The surgical device 8m may optionally be configured to deliver a heated fluid and/or heat the wound tissue. In order to maintain normothermia, warm (or hot) liquid may flow through surgical device 8m and be delivered to the wound margin. The warm or hot liquid may then heat the wound, skin, and/or adjoining tissue to a pre-determined temperature, thereby increasing the open body cavity temperature and reducing the risk of developing localized hypothermia. The fluid delivered to the surgical site (e.g. the wound) may be warmed prior to delivery or during delivery so as to maintain the body temperature of a patient. Maintenance of a patient's body temperature may lower the occurrence of and/or prevent the incubation of bacteria. Alternatively or in combination, maintenance of a patient's body temperature may reduce the amount of time needed for the wound to heal after surgery. Alternatively or in combination, maintenance of a patient's body temperature may promote wound healing and/or immune function in the patient. Alternatively or in combination, maintenance of a patient's body temperature may promote tissue perfusion. Alternatively or in combination, maintenance of a patient's body temperature may reduce or prevent the formation of necrotic tissue in the wound.

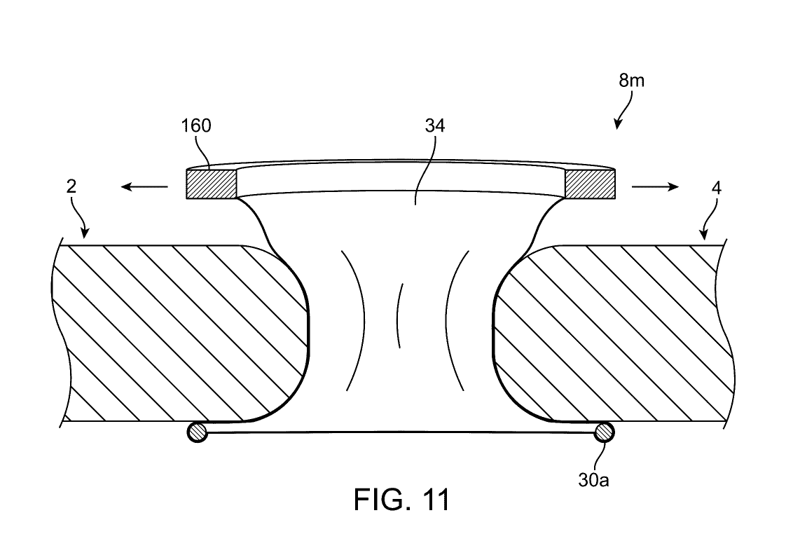

FIGS. 14A-14C show a cross-section of a surgical device 8m configured to deliver a heated fluid to the wound tissue 4. The surgical device 8m may comprise an expanding linkage structure 160, a pliable membrane 34, and a retention ring 30a. The surgical device 8m may be fluidly coupled to a fluid delivery path 1410, for example tubing, which may deliver the fluid from a fluid source 1430, for example an intravenous (IV) bag, to the surgical device 8m. The fluid may be delivered from the surgical device 8m to the wound tissue 4. Fluid may further be removed from the surgical site after irrigating the wound via the surgical device 8m and a fluid removal path 1420. The fluid may flow from the fluid source 1430 through the fluid delivery path 1410 to the surgical device 8m into the tissue 4 and out of the surgical device 8m through the fluid removal path 1420 as indicated by the arrows in FIG. 14A. The fluid may be pre-warmed prior to entering the fluid delivery path 1410. Alternatively or in combination, the fluid may be warmed within the fluid delivery path 1410 one or more heating elements 1400. FIG. 14B shows an exemplary fluid delivery path 1410 comprising a heating element 1400. The fluid delivery path 1410 may flow through the heating element 1400 (for example, the heating element 1400 may be disposed adjacent or around the fluid delivery path 1410. In some instances, it may be beneficial to increase the amount of time the fluid is exposed to the heating element 1400. FIG. 14C shows an exemplary fluid delivery path 1410 comprising a plurality of switchbacks in order to increase the length of the path 1410 in thermal contact with the heating element 1400 without increasing the size of the heating element 1400. The heating element 1400 may heat the fluid via RF via RF energy, light or IR energy, microwave energy, resistive heating, chemical heating, or any combination thereof.

The fluid may be warmed to a temperature within a range of about 35 C to about 45 C, for example about 36 C to about 42 C, preferably about 37 C. It will be understood by one of ordinary skill in the art that the fluid may be warmed to any temperature desired prior to and/or during delivery to the wound.

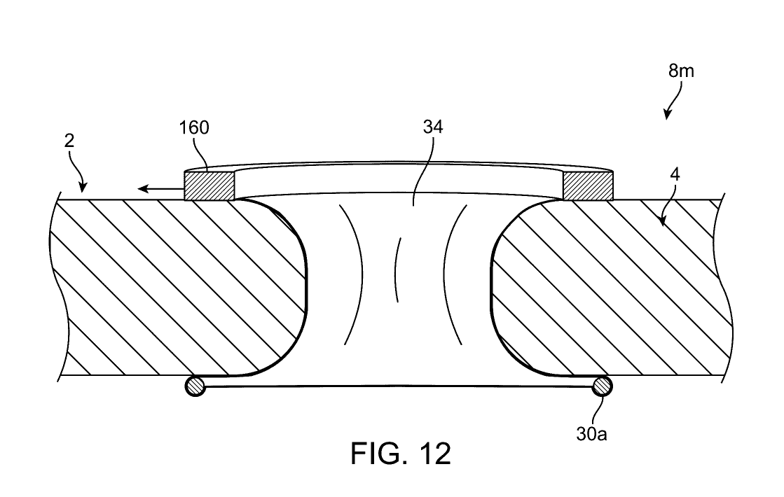

FIG. 15 shows a cross-section of a surgical device 8m comprising one or more heating elements. The one or more heating elements 1400 may be embedded within, adjacent to, and/or around the pliable membrane 34, expanding linkage structure 160, and/or the retention ring 30a. One or more heating elements 1400 may for example be disposed between the expanding linkage structure 160 and the skin 2. One or more heating elements 1400 may be embedded in or attached to the pliable membrane 34. One or more heating elements 1400 may be embedded in or attached to the retention ring 30a. The one or more heating elements 1400 may be disposed along the fluid delivery path of the fluid so as to heat the fluid during delivery. The one or more heating elements 1400 may heat the fluid via RF via RF energy, light or IR energy, microwave energy, resistive heating, chemical heating, or any combination thereof. The one or more heating elements 1400 may be integral to the surgical device 8m. Alternatively or in combination, the one or more heating elements 1400 may be separate from the surgical device 8m. For example, a heating element 1400 may be inserted along the pliable membrane 34 so as to heat the fluid as it passes through the pliable membrane 34. The heating element 1400 may be removed prior to collapsing and removal of the surgical device 8m from the wound.

Alternatively or in combination, the pliable membrane 34, expanding linkage structure 160, and/or the retention ring 30a may comprise one or more heating elements configured to heat the tissue directly. One or more heating elements may be embedded within and/or around the pliable membrane 34, expanding linkage structure 160, and/or the retention ring 30a such that they are in thermal contact (e.g. direct contact or in contact with a material capable of thermal transfer to the tissue).

Fluid Delivery

During open surgery, the skin is opened to gain access to the surgical site within the body. The opened tissue is typically exposed to the open air and the ambient environment of the surgical suite. Exposure of the wound tissue, especially at the margins, to the air may cause the tissue to lose moisture via evaporation. The surgical device 8m may be used to reduce contamination of the wound during surgery as described herein. The surgical device may cover the wound margins to reduce exposure of the wound tissue to the environment and prevent bacteria and diseased tissue from coming into contact with the wound. The surgical device may further be configured to seal the wound tissue and prevent evaporation and fluid loss from the tissue. The pliable membrane of the surgical device may comprise a fluid impermeable layer as described herein (e.g. impermeable layer 21 in FIG. 5). The fluid impermeable layer may mechanically prevent foreign liquids, including air, from contacting the wound margins as well as seal the moisture levels inside the wound margins. As surgical tools and tissue are manipulated and moved into and out of the surgical field, the wound tissue may be protected from incidental contamination during the surgery. However, bacteria may still reach the wound by circumventing the barrier as described herein.

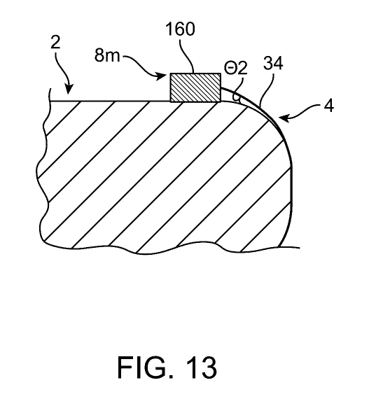

The surgical device 8m may be configured to delivery fluid to a tissue as described herein. FIGS. 16A-16C shows a surgical device 8m comprising a fluid delivery mechanism. FIG. 16A shows a cross-section of the surgical device 8m comprising fluid delivery. The surgical device may comprise an expanding linkage structure 160, pliable membrane 34, and retention ring 30a as described herein. The pliable membrane 34 may comprise a fluid delivery mechanism. The fluid delivery mechanism may comprise an active fluid delivery mechanism. Alternatively or in combination, the fluid delivery mechanism may be a passive fluid delivery mechanism. FIG. 16B shows a pliable membrane 34 configured to actively deliver a fluid 1610 to the wound 4, for example to counteract fluid loss 1600 due to evaporation. The pliable membrane 34 may comprise a plurality of perforations through which the fluid may pass to the tissue 4 as described herein. The perforations may be disposed in the layer of the pliable membrane 34 in contact with the wound tissue (for example the permeable or semi-permeable layer 25 of FIG. 5). The fluid may be delivered through the perforations to the wound tissue during surgery to reduce the contamination from bacteria which may reach the wound despite the barrier function of the pliable membrane 34 described herein. Fluid may be delivered at pre-determined time intervals or continuously throughout a surgery to reduce the contamination from bacteria that circumvented the barrier. FIG. 16C shows a pliable membrane 34 configured to passively deliver a fluid 1610. The pliable membrane 34 may comprise a sponge-like material 1620 disposed next to the wound margins 4. The sponge-like material 1620 may be configured to absorb and/or wick fluid along its surface and interior. The sponge-like material 1620 may be pre-wetted prior to insertion into the wound. Alternatively or in combination, the sponge-like material 1420 may be moistened after being placed in the wound. The sponge-like material 1420 may be an integral component of the pliable membrane 34. Alternatively or in combination, the sponge-like material 1420 may be a separate component of the surgical device 8m configured to sit between the pliable membrane 34 and the wound tissue 4.

The fluid may be delivered to the wound tissue margin 4 in order to irrigate the wound 4 and/or prevent the wound 4 from drying. Fluid may be delivered to the wound margins 4 in order to reduce tissue desiccation. Reduction of tissue desiccation or drying may reduce the time required for the wound to heal after surgery. Fluid delivery, alone or in combination with sealing of the wound tissue by the pliable membrane as described herein, may reduce or prevent tissue desiccation and/or replace any moisture lost from the tissue during surgery. Alternatively or in combination, fluid may be delivered to the wound tissue 4 in order to treat the tissue with a therapeutic agent as described herein. The fluid may comprise saline, antibiotics, or other therapeutic agents as described herein.

Alternatively or in combination, the fluid delivery function of the surgical device may be used for the cleansing of the wound surface by clearing away debris such as fat, bacteria, or other particles. Continuous delivery of a fluid to the wound surface may cause a continuous flow across the wound surface that could wash debris, bacteria, or particles off of the wound surface and into the abdominal cavity. Alternatively or in combination, the fluid removal function of the surgical device may be used to remove fluid and debris from the surgical site and reduce the accumulation of debris, bacteria, or other particles in the abdominal cavity.

Wound irrigation (e.g. fluid delivery) may be initiated by the user by manually triggering fluid delivery (e.g. by connecting the fluid source to the pliable membrane or by starting fluid flow on a pre-connected fluid source) or it may be initiated automatically (e.g. in response to the device being locked in an expanded position, etc.).

The surgical device 8m may comprise any of the features descried herein. For example, the expanding linkage structure 160 may be operable by a single user with a smooth closure motion, which may reduce splashing of bodily fluids and contamination of the wound and/or healthcare provider(s). The expanding linkage structure 160 may further be configured to reduce tissue compression as described herein. By reducing tissue compression, the amount of tension applied to the pliable membrane 34 may be reduced compared to previous designs which may result in reduced actuation forces necessary to achieve retraction of the wound 4. The larger surface area of the surgical device 8m compared to previous designs which involve rolling of the sleeve (e.g. pliable membrane) around a rolling ring with a small cross-sectional diameter may provide a greater mechanical advantage and thus allow wound retraction by a single user. These features in combination with fluid delivery and/or removal from the wound may improve efficiency of care by combining multiple features into a single device structure.

The fluid may comprise an antibiotic solution. The fluid may comprise a mixture of one or more of the following: water, saline, sterile water, Ringer's solution, Lactated Ringer's solution, Hartmann's solution, Tyrode's solution, phosphate buffered saline (PBS), gentamicin, kanamycin, antibiotics from the aminoglycoside family, or any other antibiotic effective against bacteria. The solution could comprise antibiotics with specific bacterial coverage of the various kinds of bacteria encountered during surgery including, but not limited to, enteric bacteria from the digestive tract, skin bacteria, gram-positive bacteria, gram-negative bacteria, aerobic bacteria, anaerobic bacteria, or any combination thereof. Delivery of an antibiotic solution to the wound site could reduce contamination, and subsequent infection, by neutralizing bacteria as soon as it reaches the wound tissue.

The surgical device may be used to deliver a fluid to the wound tissue and reduce or prevent contamination of the wound surface caused by enteric bacteria, skin flora, gram-positive bacteria, gram-negative bacteria, aerobic bacteria, anaerobic bacteria, and or any other microbial species known to one of ordinary skill in the art. The surgical device may be used to deliver a fluid to the wound tissue and neutralize enteric bacteria, skin flora, gram-positive bacteria, gram-negative bacteria, aerobic bacteria, anaerobic bacteria, and or any other microbial species known to one of ordinary skill in the art. The surgical device may be used to deliver a fluid to the wound tissue to cleanse the wound. The surgical device may be used to deliver a fluid to the wound tissue to clear bacteria and/or debris from the wound surface. Alternatively or in combination, at least a portion of the fluid may be removed from the wound surface by the fluid removal function of the surgical device as described herein.

Localized Therapeutic Fluid Delivery

Any of the devices described herein or in related applications may be used to intra-operatively delivery fluids to the wound site. The fluids may for example comprise one or more of a dissolved antibiotic solution, saline, Tyrode's solution, Lactated Ringer's solution, antimicrobial agents, diluted isopropyl alcohol, diluted ethanol, chlorahexainde, chlorahexadine gluconate, or other fluids commonly used to provide surgical irrigation or therapeutic benefit. The fluid may comprise one or more solute species which have been dissolved or suspended therein. Delivery of such fluids alone or in combination with fluid removal from the wound may reduce contamination beyond levels attainable with barrier protection alone. Delivery of therapeutic fluids may further reduce contamination during surgery and/or prevent growth of infectious species in the wound after surgery, thereby reducing the risk of developing surgical site infection.

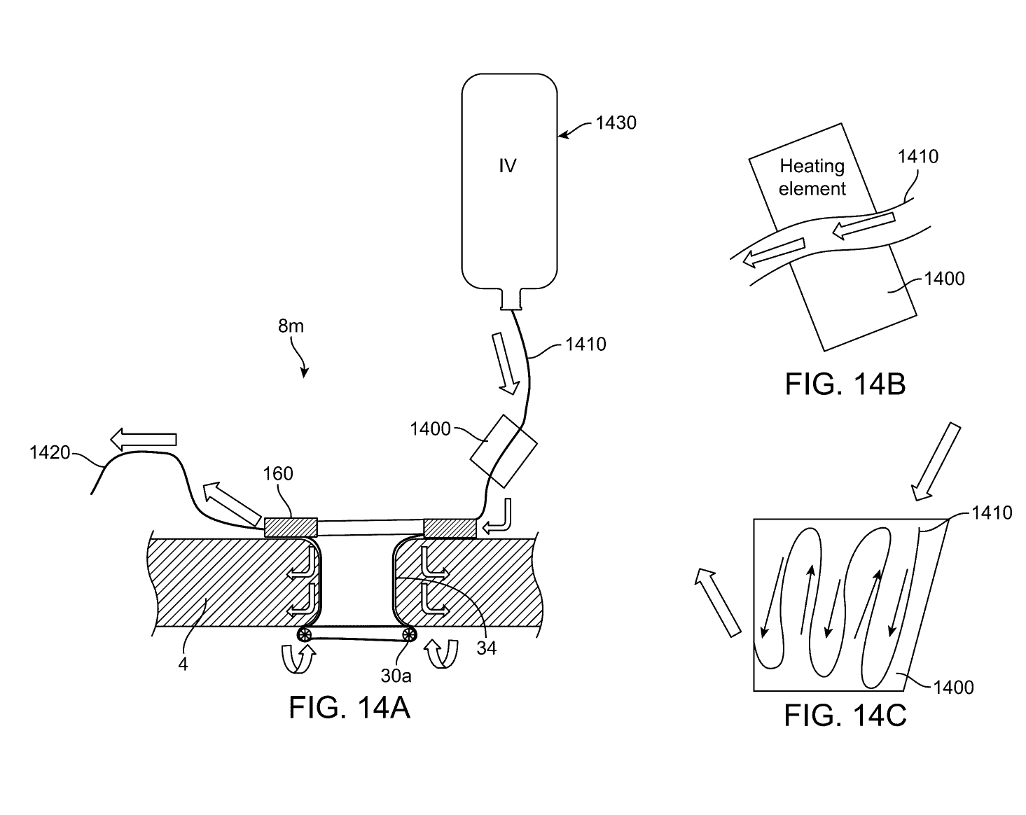

FIGS. 17A-17B show diagrams of therapeutic fluid delivery to a wound 4 to combat infection using a surgical device. During the surgical procedure, it is anticipated that the wound surfaces may become contaminated with bacteria or other organisms. The delivery of fluid to the wound margins during the surgical procedure may serve to inactivate and counteract the growth of bacteria that reach the wound margins during the procedures. Furthermore, after completion of the surgical procedure (including wound closure) the absorbed fluid and/or solute may remain in the wound and be effective to inactivate and counteract the growth of any bacteria or other organisms that may have reached the wound surface during surgery. FIG. 17A shows intraoperative delivery of a therapeutic fluid 1610 to the wound tissue 4. FIG. 17B shows post-operative action of the therapeutic agent to prevent microbial growth. The surgical device may be any of the surgical devices described herein. The surgical device may deliver a fluid 1610 to the wound 4 via a pliable membrane 34 as described herein. The fluid 1610 may for example comprise a therapeutic agent, for example a therapeutic solute 1700. The therapeutic solute 1700 may for example comprise one or more antibiotic compounds. The fluid 1610 may be delivered pliable membrane through direct contact with the wound 4. Alternatively or in combination, the fluid 1610 may be delivered into a space 1710 between the pliable membrane 34 and a margin of the wound 4. The fluid 1610 may subsequently contact the wound 4 and be absorbed by the tissue. Alternatively or in combination, the therapeutic solutes 1700 in the fluid 1610 may diffuse into the wound tissue 4 along a concentration gradient as described herein. The therapeutic solutes 1700 and/or fluid 1610 may be delivered into the interstitial space or fluid 1770 between cells 1720. The tissue 4 may retain the therapeutic solutes 1700 and/or fluid 1610 in order to combat invading bacterial species while the wound is opened or closed. Post-operatively, the closed wound 1730 may comprise a dead space 1740 located between opposing incision edges which may be filled with residual fluids and/or contaminants (such as bacteria) 1760. If a therapeutic agent was delivered to the tissue during surgery, the tissue may retain the therapeutic agent (solute or fluid) as described herein. The tissue may thus be able to counteract the growth of bacteria 1760 or other microorganisms trapped in the space 1740. The retained therapeutic agents may prevent bacteria 1760 from invading the tissue 4. Alternatively or in combination, the tissue 4 may release retained therapeutic agents 1750 into the space 1740 where they may act to inactivate or reduce the growth of bacteria 1760 in the space 1740.

The extent of absorption of a fluid or solute into the tissue may be a function of the rate of fluid delivery, concentration of solutes (if any) in the delivered fluid, physical characteristics of the solutes (if any), the porosity of the tissue, tortuousity of the tissue, systemic absorption via the capillary bed and subsequent renal clearance, and/or the time period over which the surgical device and delivered fluid are in contact with the wound tissues. For example, the local concentration of gentamicin within a tissue may be a function of distance from the wound margin or edge. The gentamicin may be delivered at a concentration of 240 mg/L to the wound surface using the surgical device for four hours. Minimum inhibitory concentrations (e.g. above about 16 mcg/ml) of gentamicin solute may be found approximately 3.8 millimeters (mm) into the tissue. The minimum inhibitory concentration (16 mg/L) was reached are the wound surface in about 3 minutes of fluid delivery. The minimum inhibitory concentration was reached at approximately 3.8 mm tissue depth within about 4 hours of fluid delivery. Upon removal of the surgical device, the gentamicin may be retained in the tissue at minimum inhibitory concentrations for a period of about 3 to 4 hours after wound closure. The surgical device may thus be able to reduce microbial growth in wounds even after being removed, which may result in a lower risk of developing a surgical site infection.

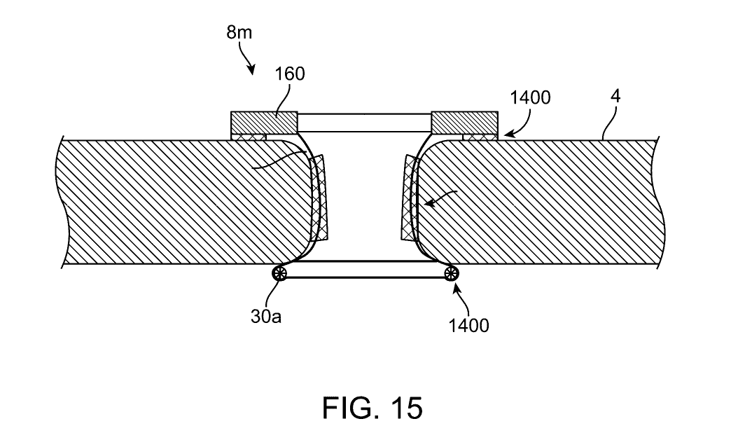

Local delivery of therapeutic agents, for example using any of the surgical devices described herein, may be preferable to systemic delivery in at least some instances. In some instances, the therapeutic agent delivered may be difficult to deliver systemically or may have undesirable side-effects which may limit dosing when delivered systemically. For example, gentamicin is associated with known oto-toxic and nephron-toxic side-effects which may limit the dosage that can be delivered via intravenous or other systemic administration methods. Limiting the dose of gentamicin given to a patient in order to reduce side effects may reduce the effective concentration at the wound due to systemic circulation and delivery to locations other than the wound, which may limit the ability of the antibiotic to inactivate and counteract bacterial growth in the wound. For example, a bolus intravenous injection of 100 mg gentamicin may result in maximum blood serum concentrations of up to 6 mg/L (e.g. mcg/ml) in healthy patients. The blood serum concentration may decrease exponentially after the initial dose as shown in FIG. 18 and would be further diluted at the wound surfaces due to diffusion through the tissue from the vasculature. Thus, systemic administration of gentamicin may require undesirably high systemic concentrations in order to achieve local tissue concentrations which may prevent surgical site infections. Systemic administration of therapeutic agents such as antibiotics may alternatively or in combination expose the entire patient's microbiome to the therapy which may increase the patient's risk of developing antibiotic-resistant bacterial strains and negatively affect the patient's health as a consequence. Local delivery, on the other hand, may provide the therapeutic agents at or near the wound surface in concentrations which may be effective to inactivate and/or prevent growth of the bacterial species without subjecting the patient to the potential side effects associated with systemic delivery. Alternatively or in combination, the therapeutics agents may be absorbed or diffused into the wound tissue as described herein, effectively creating a local reservoir of therapeutic agent within the tissue which may work to reduce bacterial growth even after the surgical device and associated fluid delivery functionality have been stopped or removed from the surgical site and/or the wound has been closed. This functionality may be beneficial for packed or open wounds that are not closed after surgery due to certain known patient risk factors, providing much needed additional prophylaxis to a wound that may be severely compromised. The total local dose of the therapeutic agent may be reduced or minimized using local delivery methods. Additionally, local delivery methods may reduce the system concentration of the therapeutic agent as systemic absorption through the tissue capillary bed may occur relatively slowly.

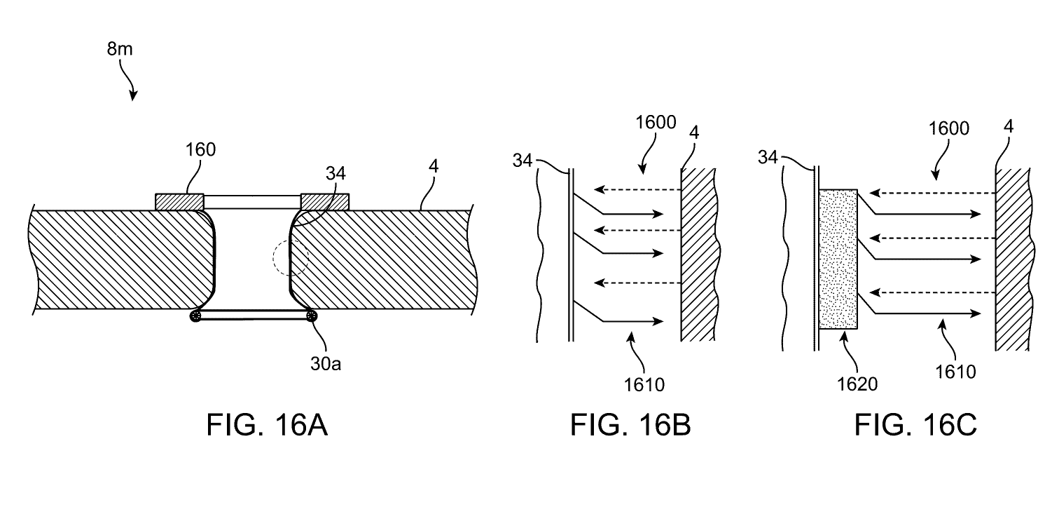

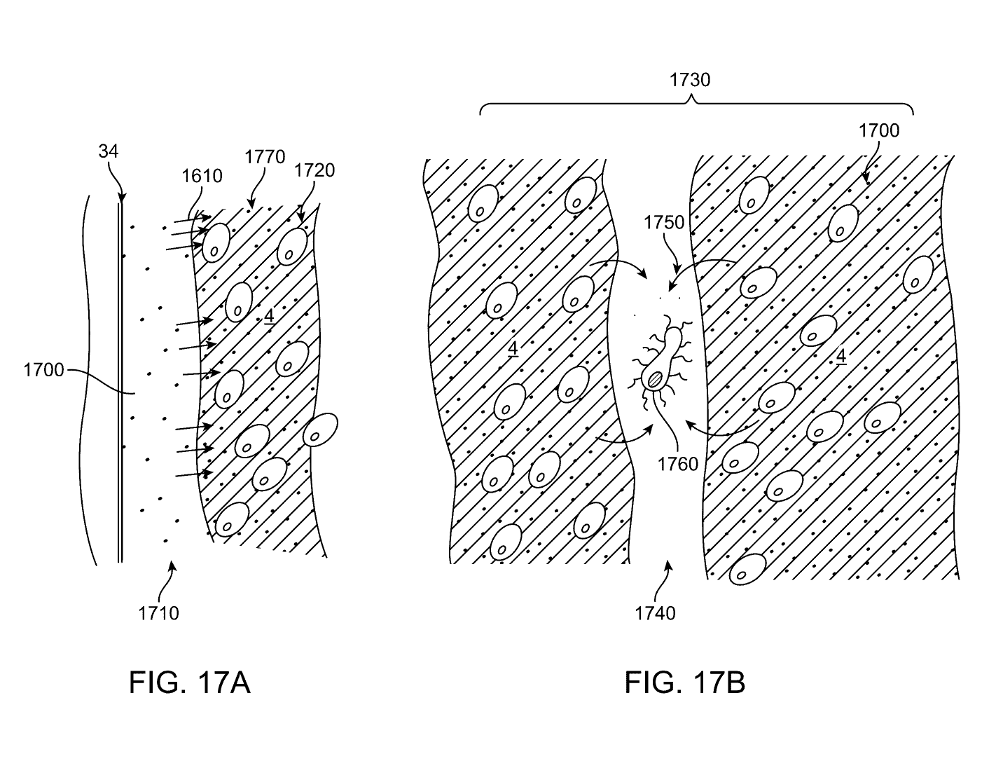

FIG. 19 shows a schematic of systemic antibiotic delivery compared to local fluid delivery using the surgical device over time. Current surgical practices may include administration of a dose of antibiotic intravenously prior to surgery. Such delivery relies on the transport and diffusion of the antibiotic throughout the body to reach the wound site to achieve a therapeutic level (e.g. a minimum inhibitory concentration) against the target bacteria. The antibiotic must diffuse from the bloodstream out through the subcutaneous tissue to reach the wound site, which requires a greater concentration in the bloodstream than in the tissue to provide the necessary gradient to achieve a sufficient concentration in the tissue and has a significant delay between delivering the antibiotic to the blood stream and the antibiotic reaching the tissue. This may require dosing a patient prior to surgery and/or during surgery. In many cases, an initial dose may be given to the patient prior to surgery in order to attain therapeutic levels in the wound. Follow-up maintenance doses 1920 may be given periodically in order to maintain the antibiotic concentration at or near the therapeutic level. FIG. 20 shows a schematic representation of tissue antibiotic concentration with periodic intravenous infusions. This may require precise timing to achieve the right antibiotic concentration at the right time (during surgery) in the right place (at the surgical site) due to the fact that the body begins filtering the antibiotic out of the blood as soon as it is delivered to the bloodstream (1910 of FIG. 19 shows the changes in concentration which would occur over time in no maintenance doses were given). This may result in fluctuating levels of antibiotic 1930 in the system (FIG. 19) and the wound (FIG. 20) which may impair therapy. Bolus injections at the wound site may have similarly fluctuating levels and may require maintenance dosing to achieve a therapeutic concentration. The variability of surgery timing and duration and/or unexpected delays may make it very difficult to achieve and maintain substantially constant antibiotic levels at or above the minimum inhibitory concentration during the course of a surgery. Multiple injections of a precise amount of antibiotic may need to be implemented to account for timing changes when using systemic delivery methods. Further, it may be difficult to maintain the therapeutic concentration in the wound without exceeding toxic levels systemically for antibiotics with known toxicities. Local delivery of the antibiotic fluid to the wound, using the surgical device described herein instead of systemic delivery may avoid the challenges of systemic delivery while providing higher local concentrations than may be possible to achieve with systemic delivery when toxicities are of concern. Local delivery of the antibiotic fluid to the wound may allow the concentration at the wound margin to remain constant throughout the duration of delivery which may be beneficial compared to the fluctuating concentrations of systemic delivery. Additionally, the fluid may be delivered during the entire surgery or any portion thereof and may reach therapeutic concentrations nearly instantly, thereby providing a time-insensitive method of antibiotic delivery. Therapeutics may be delivered directly to the site of interest (no need to wait for accumulation) and at the time it's needed (intraoperatively). Such an approach might be termed “on demand” therapeutic delivery. This may be desirable as delivery may be independent of delays or changes in surgical schedules. Delivery may be provided independent of delays or changes in surgery timing or duration as the antibiotic delivery may not start until the surgery begins and can be extended indefinitely by providing additional fluid (e.g. another fluid bag or source) to continue the antibiotic delivery if the surgery were to extend longer than anticipated.

The surgical device may be used to deliver a therapeutic fluid to a wound tissue to maintain a constant therapeutic concentration in the tissue. The therapeutic concentration in the tissue may be maintained for a portion of or the entire duration of the surgical procedure. The concentration of the therapeutic agent (e.g. antibiotic) may be maintained within a pre-determined range. The pre-determined concentration may depend on the type of therapeutic used as described herein. For example, the concentration of gentamicin within the tissue may be maintained within a of about 16 mg/L to about 250 mg/L to treat E. coli as described herein. The therapeutic concentration may be at or above the minimum inhibitory concentration. The therapeutic concentration may be within a range, for example within about +/−1 mg/L of the minimum inhibitory concentration. The therapeutic concentration may be within a range of +/−5%, 10%, 15%, 20%, 25%, 30%, 35%, 40%, 45%, or 50% of the minimum inhibitor concentration. It will be understood by one of ordinary skill in the art that the therapeutic concentration delivered may depend on the species being targeted or other factors described herein or known in the art. The therapeutic concentration may be reached more quickly than is possible with systemic delivery methods. The concentration of the therapeutic agent may be higher at the wound than in the bloodstream. Delivering the therapeutic agent directly to the wound may reduce potential risks associated with high systemic concentrations. The surgical device may be used to deliver a therapeutic fluid to a wound tissue to achieve a desired therapeutic concentration at the wound surface without the therapeutic fluid passing through the circulatory system. The surgical device may be used to deliver a therapeutic fluid to a wound tissue to achieve a desired therapeutic concentration at the wound surface without systemic diffusion.

The therapeutic concentration may be determined based on the therapeutic agent being delivered. For example, the therapeutic concentration of gentamicin may be within a range of about 100 mg/L to about 400 mg/L, for example about 240 mg/L. The therapeutic concentration of metronidazole may be within a range of about 250 g/L to about 7500 g/L, for example within a range of about 500 g/L to about 5000 g/L. The therapeutic concentration of clindamycin may be within a range of about 300 mg/L to about 1000 mg/L, for example about 640 mg/L. The therapeutic concentration of neomycin may be within a range of about 20 mg to about 60 mg, for example about 40 mg. The therapeutic concentration of polymyxin B sulfate may be within a range of about 100,000 units to about 300,000 units, for example about 200,000 units. It will be understood by one of skill in the art that any therapeutic concentration may be delivered as desired as the therapeutic concentration needed to target a desired microorganism may differ between therapeutic agents due to any of the factors described herein or known in the art.

The therapeutic fluid delivered locally may be more concentrated than would be possible to deliver intravenously, thereby achieving higher tissue concentrations levels than intravenous routes if desired for high-risk patients as intravenous concentrations may be limited by the risk of systemic toxicity. The bloodstream concentration could be monitored and used as a feedback mechanism to adjust the antibiotic concentration delivered to the wound in order to ensure a safe bloodstream level is maintained at all times. Several patient characteristics, such as Body Mass Index (BMI) or other factors described herein, may affect the distribution of an antibiotic fluid in the wound tissue. Such characteristics may be difficult to estimate and adjust for when using a systemic delivery approach. A local delivery approach can be applied directly to the wound tissue for all patients and may reliably achieve a therapeutic concentration of antibiotic in the tissue as dosing may be quickly and easily tuned to the needs of the individual patient. Alternatively or in combination, local delivery of the antibiotics may result in lower amounts of systemic absorption which may reduce the risk of developing antibiotic resistance compared to systemic delivery methods as the therapeutic agent may only be present in the wound tissue at meaningful concentrations.

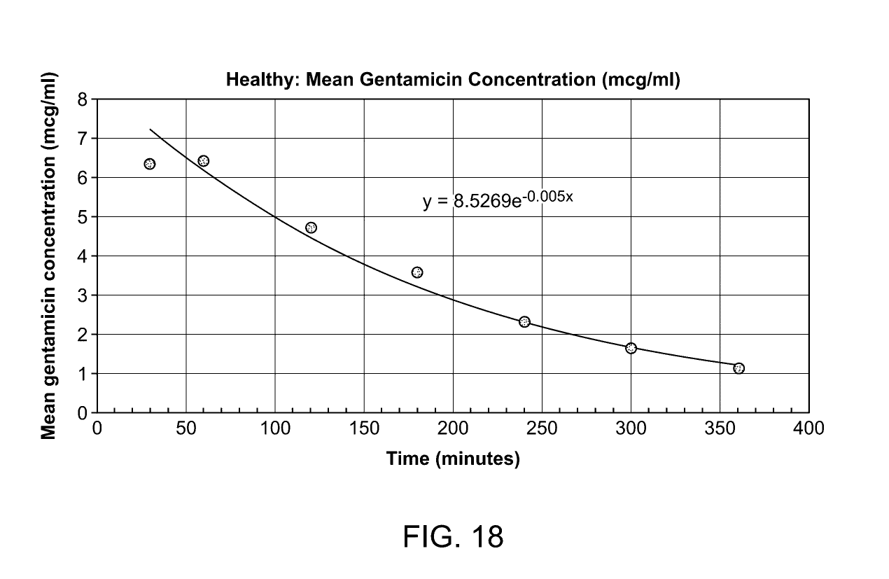

FIG. 21 shows differences in antibiotic concentration between the tissue and the bloodstream when locally delivered over time. An antibiotic fluid was delivered to porcine wound tissue at a concentration of 240 mg/L for 4 hours using the surgical device as described herein. Three female adult pigs were treated as described in Experiment 3, with one pig in each of the treatment groups (control, E-NF, and E-F). It was expected that over the course of the procedure the antibiotic would slowly diffuse into the tissue of the pig receiving antibiotic fluid (E-F) and, eventually, reach the systemic circulation. The concentrations of the antibiotic in the bloodstream and in the wound of the E-F pig were measured following removal of the surgical device and 4 hours after. The concentration of the antibiotic was higher in the tissue than in the bloodstream at both time points assessed. Further, the tissue retained a portion of the delivered antibiotics at 4 hours after treatment stopped, indicating that local delivery may have beneficial effects even after removal of the source of antibiotic.

### Experiment 2

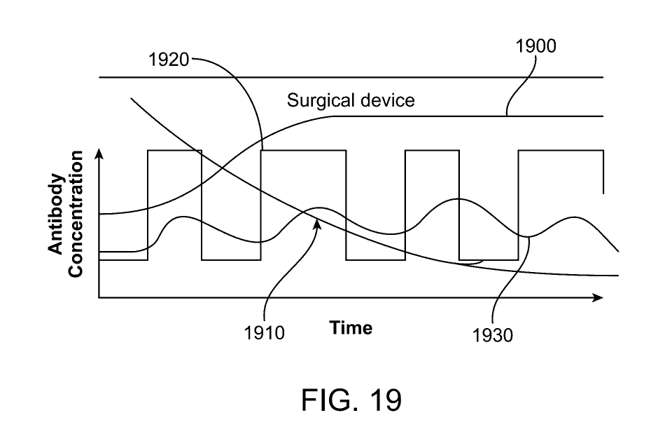

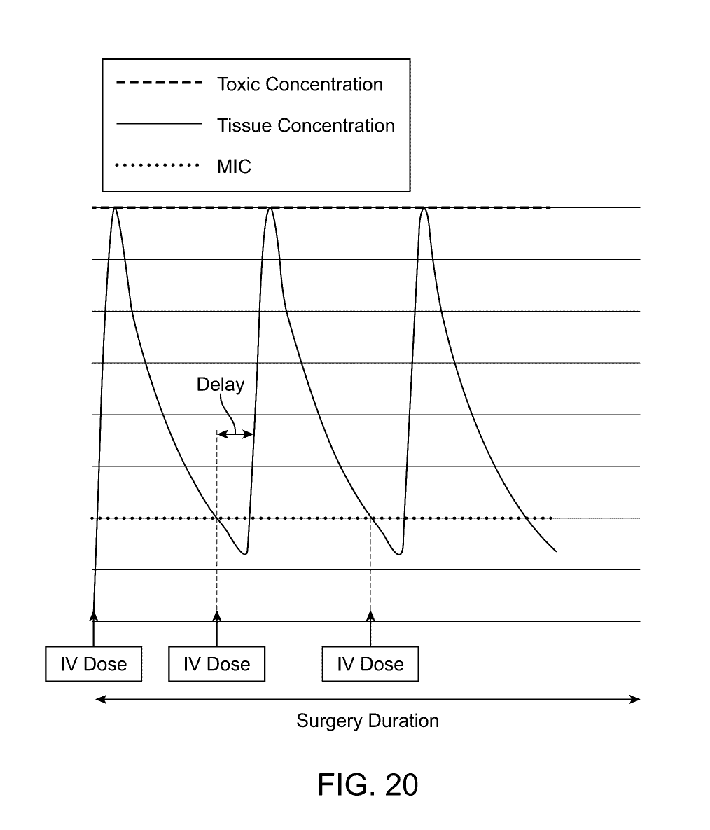

In order to better understand the differences between systemic delivery and local delivery of therapeutic agents, a finite-element analysis software package (COMSOL) was used to generate a three dimensional (3D) model of a retracted surgical wound with a 12 cm incision. FIG. 22 shows the surgical device 8m comprising an expanding linkage structure 160 and pliable membrane 34 with fluid delivery as described herein inserted into a wound created in a cadaver model. The 3D model shown in FIG. 23 was modeled in a Computer Aided Design (CAD) package (Solidworks) based on the geometry of the device and the retracted wound tissue in a cadaver.

Gentamicin, a member of the aminoglycoside family, was used as the antibiotic in the model due to its prevalence in clinical use to combat the enteric, gram-negative bacteria that cause wound infections. A concentration of 240 mg/L was delivered during the simulated 4 hr surgery based on standard clinical protocols.

Model Parameters

Diffusion

The classical differential equation that describes the diffusion of particles into a solution was first described by Adolf Fick, and is known as Fick's Law as shown in Equation 3,

Φ=−D∇C (3)

where Φ, D, and C are the particle flux, diffusion coefficient, and concentration, respectively. This relation may be extended to apply to porous media, such as biologic tissues, by accounting for the effect of the relevant impacts of the specific tissue. The diffusion of solutes in subcutaneous wound tissue may be subject to greater resistance due to the decreased volume into which the solute can diffuse (porosity) as well as the restricted flow paths that the solute particles can follow (tortuosity) due to the presence of the adipocytes that comprise the tissue. Therefore, the diffusion coefficient in a porous medium may be adjusted to account for these effects. Equation 4 can be to calculate an “effective” diffusion coefficient,

$${D}_{eff} = \frac{D}{{λ}^{2}} \qquad ( 4 )$$

where λ is the tortuosity of the tissue and D is the diffusion coefficient in the pure solution. The tortuosity can be described in terms of the porosity (ε) of a closely-packed bed of cells according to Archie's Law as shown in Equation 5.

$$λ = \frac{1}{{ε}^{n}} \qquad ( 5 )$$

This may be further expanded with experimental data to reach a relation in a tissue with a “topologically dense arrangement” of cells as shown in Equation 6,

$$λ = \frac{1}{{ε}^{0.23 + 0.3 \cdot ε + {ε}^{2}}} \qquad ( 6 )$$

where ε is the porosity of the tissue. Therefore, the effective diffusion coefficient can be calculated based on the porosity of the tissue alone. The porosity of a tissue is a non-dimensional ratio of the volume of extracellular fluid divided by the total volume of the medium as shown in Equation 7.

$$ε = \frac{{V}_{fluid}}{{V}_{total}} \qquad ( 7 )$$

While this is a three-dimensional (3D) parameter by definition, it has been shown that the ratio is the same in two dimensions as three. Therefore, a two-dimensional slice can be used to ascertain the porosity of a material using image analysis of a slice of tissue.

System Absorption

As the antibiotic diffuses into the tissue, the capillaries in the tissue will absorb the antibiotic into the blood stream, being driven by the same passive concentration gradient that drives the diffusion into the tissue. Steady-state diffusion of a particle through a membrane can be characterized macroscopically by Equation 8,

Φ=P·(ΔC) (8)

where P is the permeability of the capillary to the given solute and ΔC is the concentration difference across the membrane. The permeability term accounts for the combined effects of the hindered diffusion through the membrane as well as the thickness of the membrane itself.

Parameter Calculation

Diffusion Coefficient

The diffusion coefficients of various solutes and solvents have been experimentally obtained and published. However, for solute-solvent combinations that have not been published or experimentally determined, the molecular weight of a given solute may be correlated to the diffusion coefficient in the same solvent using a logarithmic relation. The interstitial fluid that resides in between adipocytes in wound tissue is comprised primarily of water therefore, the diffusion of gentamycin in water is representative of its diffusion coefficient in interstitial fluid and can be used to calculate the baseline diffusion coefficient. The porosity of adipose tissue was obtained using image analysis (ImageJ) of a stained slide of adipose tissue.

Capillary Permeability

The capillary permeability value was obtained from experimental capillary diffusion data for glycerol in subcutaneous tissue. Glycerol has a similar molecular mass as gentamicin and may, therefore, be representative of gentamicin diffusion across a capillary. This data was combined with the ratio of capillary surface area to tissue volume for fat tissue to produce the final permeability value.

Table 3 shows the calculated model parameters used in the finite-element analysis of gentamicin delivery to a wound.

TABLE 3
Calculated Model Parameters

| Parameter | Value |
|---|---|
| Effective Diffusion Coefficient | 1.88E−6 cm²/s |
| Capillary Permeability | 3.14E−4 1/s |

Model Constraints

The model used a surface concentration on the inner surfaces of the wound to model the fluid contact and a no flux boundary condition the outer edges of the wound model, which were far enough from the fluid source that it would not affect the diffusion. The diffusion coefficient was applied to the entire tissue domain. The capillary permeability was included as a reaction term that consumes the antibiotic in the entire volume of tissue according to Equation 8.

Model Mesh

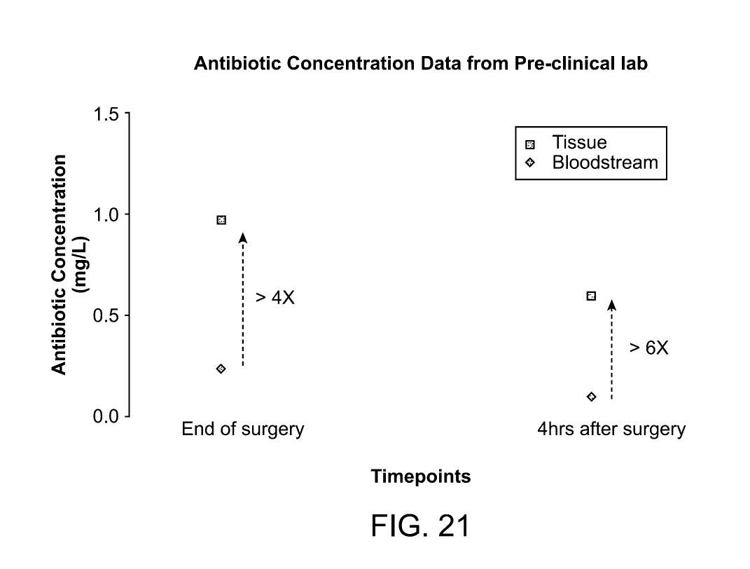

The model shown in FIG. 23 was meshed with a coarse mesh on the periphery of the model and a refined mesh near the inner surface of the wound to ensure a smooth concentration gradient as shown in FIG. 24.

Simulation Results

The simulation was run for four hours to mimic a standard colorectal procedure and the antibiotic flux and tissue concentration was recorded at each time step.

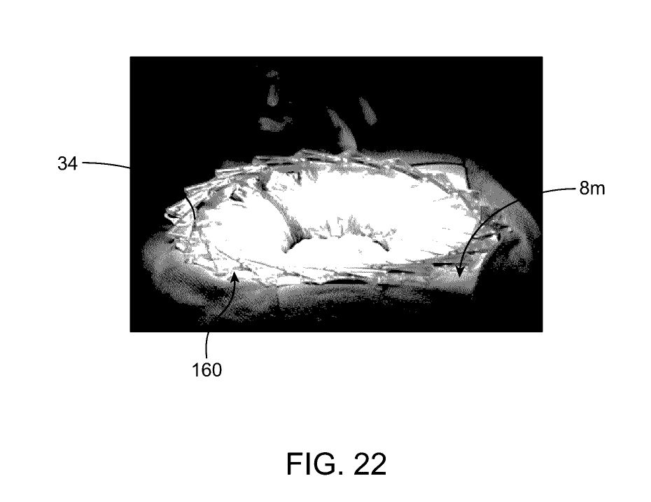

FIG. 25 shows the results of the simulation of antibiotic concentration within the tissue at 1 mm deep over the four hour simulated period. Simulated tissue concentration results are shown for local (topical) delivery of gentamicin compared with data from the literature of measurements of gentamicin concentration in a tissue following a single intravenous dose. The minimum inhibitory concentration (MIC) of 0.003 mol/m³, which is the minimum inhibitory concentration required to neutralize gram-negative bacteria present in colorectal surgeries, is shown for reference. The concentration 1 mm from the wound edge was extracted at all time points and plotted with the tissue concentration data obtained with a standard intravenous dose (240 mg) administered before the surgery. The antibiotic was shown to be present up to a distance of 3.8 mm at the minimum inhibitory dose. The tissue concentration exceeded the minimum inhibitory concentration after 6 minutes of application and reached a maximum value 46 times greater than the minimum inhibitory concentration. The concentration of the antibiotic in tissue at a depth of 1 mm from the surface rapidly increased over the minimum inhibitory concentration level and approached a steady state value within 2 hours after surgery. With intravenous administration however, the concentration reached a peak value of 4.5 times greater than the minimum inhibitory concentration roughly 20 minutes after the antibiotic administration and decreased over time as the gentamicin was filtered out of the bloodstream. The model additionally only required a cumulative dose of 14.3 mg of gentamicin to be delivered compared to the 240 mg intravenous dose.

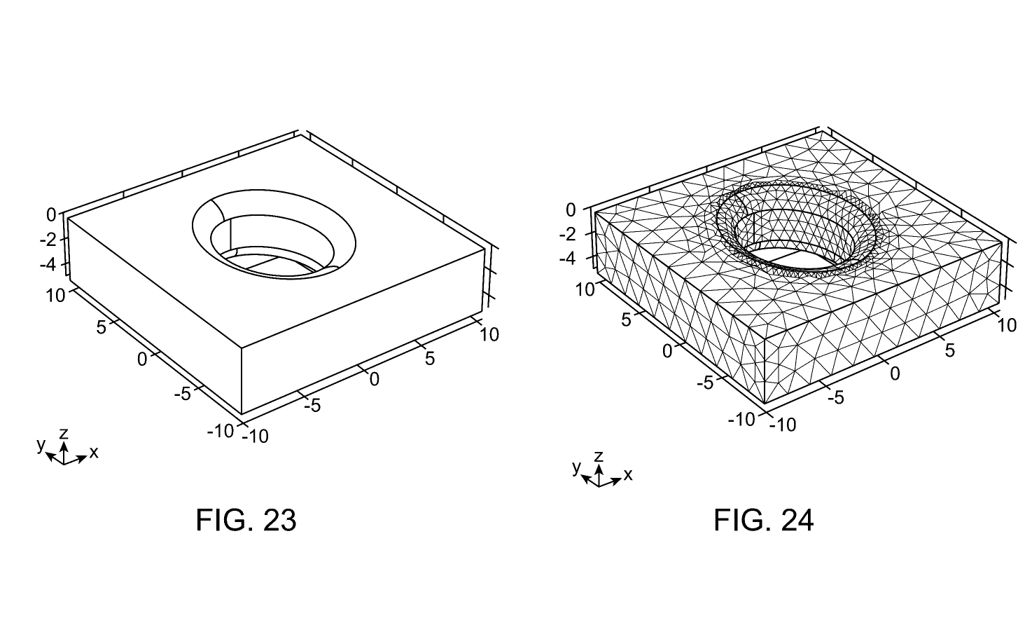

FIG. 26 shows the results of the simulation of antibiotic concentration within the bloodstream over the four hour simulated period. Simulated blood serum concentration results are shown for local (topical) delivery of gentamicin compared with data from the literature of measurements of gentamicin concentration in a tissue following a single intravenous dose. The concentration in the blood was extracted at all time points and plotted with the blood concentration data obtained with a standard intravenous dose (240 mg) administered before the surgery. As the gentamicin diffuses out of the tissue, it enters the bloodstream to be filtered out by the kidneys. Gentamicin is nephrotoxic at a peak bloodstream concentration of 12 mg/L, thus it may be desirable to maintain therapeutic concentrations at the wound while minimizing the concentration in the blood. The concentration of gentamicin in the bloodstream following intravenous injection showed an initial, significant spike above the toxic threshold which persisted for about 40 minutes until enough of the gentamicin was filtered out of the blood by the kidneys. Local delivery showed blood concentrations well below the toxic threshold (and the intravenous concentrations) for the entirety of the simulated surgery. These results suggest significant benefits to patients may be achieved with local fluid delivery by providing a significantly higher local concentration (and lower risk of infection) in combination with a lower systemic concentration (and lower risk of toxicity or resistance). The results from the tissue concentration data also demonstrate that a given concentration can be targeted based on the concentration of antibiotic in the fluid. This range could be maintained in a range of 1 mg/L, 10 mg/L, or 100 mg/L.

It will be understood by one of skill in the art that the model described herein may be tailored to account for any number of surgical situations. For example, the fluid delivered by the surgical device may contact both the wound surface and the contents of surgical site (e.g. the abdominal cavity), thereby effectively working to inactivate and/or counteract the growth of the target microbial species colonizing the surgical site. Alternatively or in combination, external sources of infection such as gloves or surgical tools may come into contact with the fluid delivered to the surgical site which may further inactivate and/or counteract the growth of microbes on these surfaces and reduce the risk of developing a surgical site infection.

In some instances, it may be desirable to tailor the delivery of an antibiotic fluid in order to limit the bacterial concentrations in the wound to 10⁴ colony forming units (CFU) per gram or CFU/ml or below. Reducing the concentration below 10⁴ CFU/g may reduce the risk (or rate) of surgical site infection.

### Experiment 3

The anti-contamination effects of the surgical device both with and without gentamicin antibiotic irrigation delivered through the device were tested in an acute abdominal surgical model in seven adult pigs. In the gentamicin group, the degree of systemic absorption of the antibiotic was quantified.

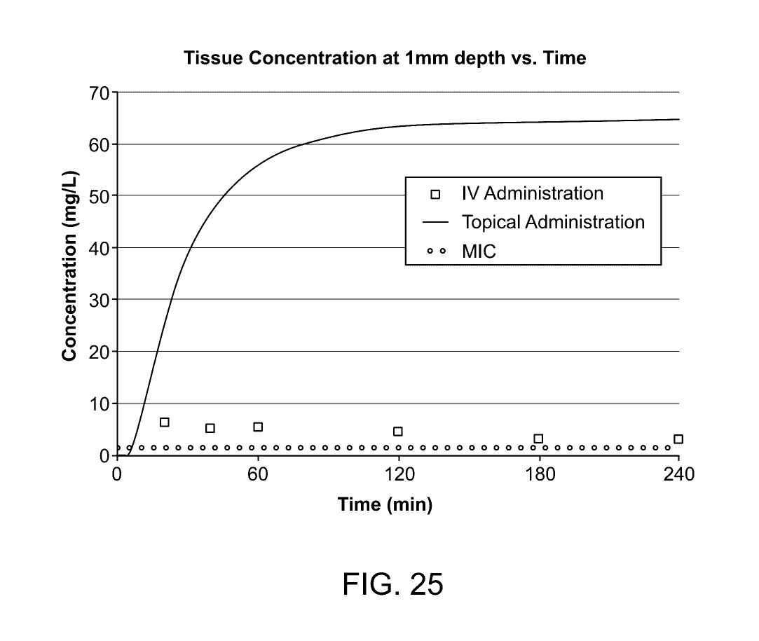

Seven female adult pigs weighing 50-70 kg were placed under general anesthesia and monitored for the duration of the experimental protocol. The abdomen was shaved, sterilely prepped with chlorhexidine solution, and draped under sterile conditions. A 12-cm periumbilical midline incision was made and the abdominal cavity was entered. The surgical device was placed in the wound, and expanded to open and retract the wound edges as shown in FIG. 27. After approximately 2.5 hours of wound retraction to simulate a prototypical length of time for colonic dissection, a gentamicin-sensitive E. coli suspension at a standardized concentration of 10⁹ colony-forming units (CFU) in 10 mL normal saline was dripped onto the central colon. E. coli is classified as an enteric bacteria species. The colon was then grasped with a Babcock clamp, exteriorized out of the abdomen and allowed to rest on the superior, left, inferior, and right aspects of the incision, respectively, for 20 minutes on each location. This set of maneuvers simulated the contamination that occurs during colonic division and anastomosis. The abdominal cavity was then irrigated with 1 L normal saline, and the surgical device was removed. Fascial closure was performed with a 0 PDS monofilament running suture. The skin was closed with staples. The pig was then kept anesthetized with the wound closed for 4 hours. The wound was then reopened, including the skin and fascia, for additional experimental data collection prior to sacrifice as described below.

The 7 pigs were divided into two groups each: 1. Control (n=1) where no device was used and a sham procedure was performed. 2. “Experimental-No Fluid” (E-NF) group (n=3) where the surgical device was used without any irrigation delivered through the device in order to mimic conditions with other non-irrigating surgical retractors. 3. “Experimental-Fluid” (E-F) group (n=3) where a solution of 240 mg gentamicin sulfate in 1 L normal saline was delivered through the surgical device to the circumference of the retracted wound at a rate of approximately 5 mL/min (per the design specifications of the device). The irrigation fluid was delivered via gravity drainage from an IV bag hung on a pole at a height of 6 feet, and removal of fluid from the device was achieved via wall suction into a standard surgical canister. Both the fluid delivery and fluid removal mechanisms were integrated into the surgical device via separate tubing connections. The total time of irrigation averaged approximately 4 hours.

Experimental data was collected at two time points. Time 1 was after 4 hours of simulated surgery and/or irrigation and at the time of device removal. Time 2 was 4 hours after wound closure (e.g. at the time of wound reopening).

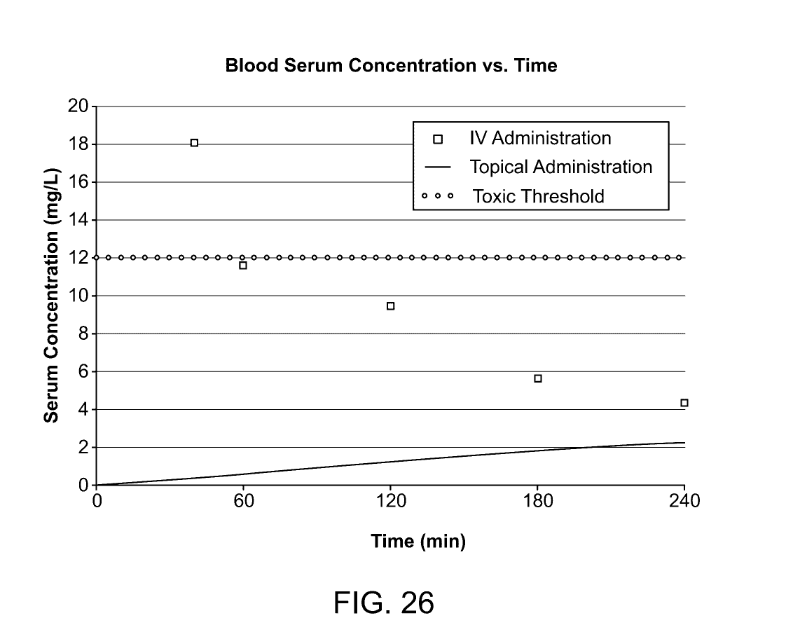

FIGS. 28A-28B show the locations at which two swab samples were taken just prior to device removal (e.g. at Time 1). FIG. 28A shows the “exposed” swab which was taken along the entire circumference of the side of the pliable membrane exposed to the surgical environment. FIG. 28B shows the “protected” swab which was taken along the entire circumference of the retracted and protected wound which was in direct contact with the pliable membrane during the simulated surgery. The swabs were placed in sterile transport tubes with PBS and stored on ice until analysis. Culture-based analysis was performed. Briefly, the tissue and swab samples were vortexed in PBS, and serially diluted onto places in duplicate on TSA media. The plates were incubated at 30-35 degrees C. for 18 hours. Colonies were identified as E. coli, enumerated, and recorded, and the results for serial dilutions were internally validated. One colony was Gram-stained to confirm that the organism was a Gram-negative rod. Tissue culture results were expressed as colony-forming units (CFU) per gram.

For tissue evaluation, a 1 cm×1 cm full-thickness abdominal wall tissue block (from skin to peritoneum) was excised at the midpoint of the incision at each time point (taken from opposite sides of the incision per each time point). The tissue samples were stored in sterile centrifuged tubes on ice until analysis.

In the E-F group, peripheral blood samples were drawn by the anesthetists at Times 1 and 2. The blood was collected in serum-separator tubes and centrifuged. The serum was isolated, serially diluted, and analyzed with an indirect-competitive gentamicin ELISA kit for quantitation of gentamicin concentration. In addition, punch biopsies were taken of the subcutaneous fat at Times 1 and 2 in a preliminary experiment to detect gentamicin levels in tissue using ELISA. Two 4 mm×8 mm punch biopsies at each time point were taken, homogenized, and diluted in 1 mL phosphate buffered saline (PBS). The sample was centrifuged, and the supernatant was isolated for ELISA analysis.

Quantitative culture results were expressed in mean±standard deviation (SD). Statistical comparisons of quantitative culture results were performed using the Student's t-test. Significance was determined based on a value of p<0.05.

Results

Swabs of the exposed side of the device sheath, representative of intraoperative (pre-closure) contamination led to high levels of E. coli growth, with a mean of 1.68±1.71×10⁴ CFU/swab. The protected side showed exponentially lower levels of E. coli growth, with a mean 1.00±0×10² CFU/swab).

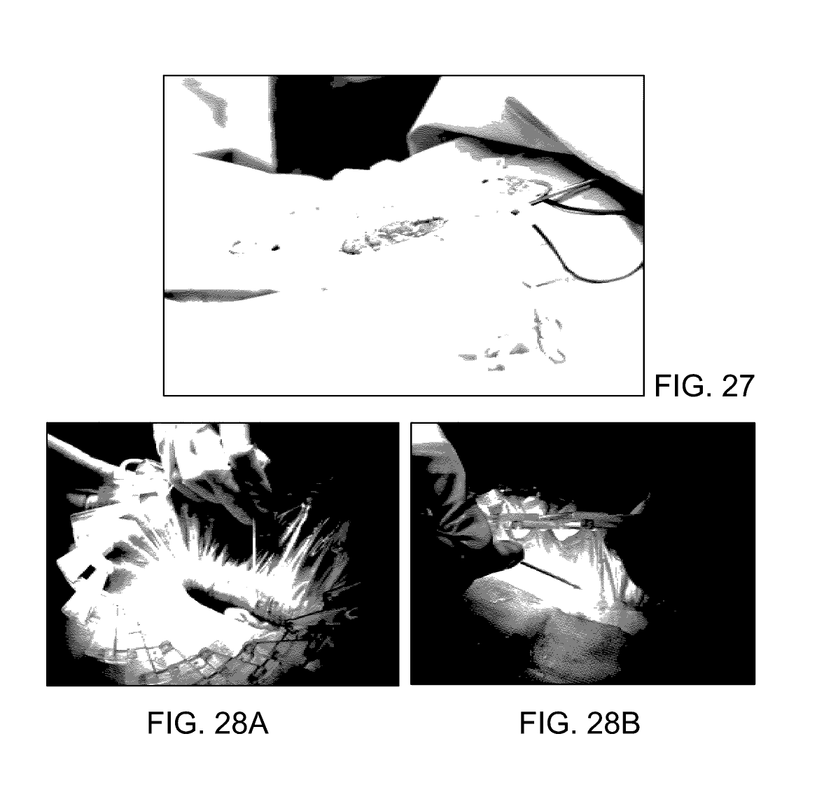

FIG. 29A shows results of comparison testing between surgical device with and without gentamicin delivery. Use of the surgical device, both with and without antibiotic irrigation, was associated with an exponential reduction in quantitative wound culture (measured via the collected tissue sample), compared to the non-surgical device sham control. Both the E-NF and E-F groups had minimal bacterial growth at Time 1 (2.04±0.61×10² CFU/gm in E-NF and 1.25±1.55×10² CFU/gm in E-F, respectively). However, the E-NF group developed exponential bacterial growth at Time 2 (2.60±1.41×10⁴ CFU/gm) compared to Time 1, the difference of which was statistically significant (p=0.041). The E-F group, on the other hand, had a durable, sustained suppression of bacterial growth at Time 2 (2.08±3.01×10² CFU/gm). The difference between E-F and E-NF at Time 2 was statistically significant (p=0.041).

FIG. 29B shows the serum gentamicin concentrations at Time 1 and Time 2 for the E-F treatment group. In the E-F group, systemic gentamicin absorption remained minimal and began to decrease at Time 2. The mean serum gentamicin concentrations were 0.24±0.11 mg/L and 0.10±0.05 mg/L at Times 1 and 2, respectively. It should be noted that the clinically-relevant threshold for serum gentamicin concentration is 1 mg/L.

FIG. 29C shows the tissue gentamicin concentrations at Time 1 and Time 2 for the E-F treatment group. Gentamicin concentrations at the wound margins Times 1 and 2 were 0.97±0.33 mg/kg and 0.60±0.37 mg/kg, respectively, which are 4-6× the corresponding serum levels, and the true concentration of gentamicin closer to the cut surface of the wound was likely far higher (approaching the 240 mg/L of the delivered fluid). It should be noted that because the bactericidal interface between the gentamicin fluid and E. coli may be primarily at the surface of the cut incision, the functional concentration of gentamicin may be by definition 240 mg/L (the dose of the delivered fluid), which is about 60 time greater than the typical minimum inhibitory concentration of E. coli. This relevant concentration may explain the significant inhibitory effect on bacterial growth seen in the tissue culture results.

In summary, use of the surgical device was associated with a significant decrease in bacterial contamination at the incision at the time of closure, and the addition of gentamicin led to an additional exponential benefit that was sustained 4 hours after closure. Serum gentamicin absorption was minimal at its peak.

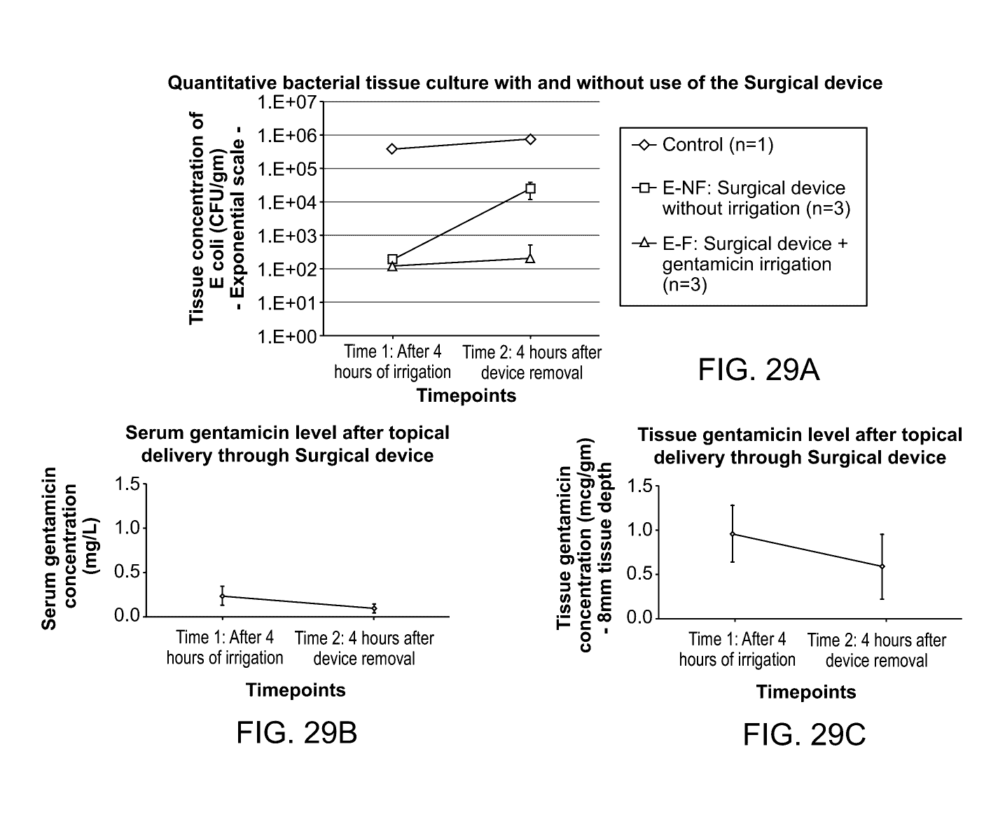

FIG. 30 shows a hematoxylin and eosin-stained tissue section collected from a wound in order to assess the extent of tissue damage with fluid delivery. Fluid was delivered to the wound margins of a porcine model at a rate of 13 mL/min. No tissue injury was observed with microscopic evaluation. Control of the fluid delivery and removal functions may be critical to implement the proper therapy to reduce bacterial contamination and reduce the risk of developing infection while minimizing the risks of systemic absorption. Tissue irritation may be considered as well, since the delivered fluid may remain in contact with the tissues of the surgical incision for an extended period of time. Too high of a flow rate could lead to skin leakage or pooling of fluid within the abdominal cavity and/or pressure dissection. Too low of a flow rate may result in inadequate absorption or diffusion of therapeutic species into the wound tissue. Similarly, too low of a fluid removal rate may result in pooling of fluid within the abdominal cavity or skin leakage, while too high of a fluid removal rate could result in tissue injury or inadequate absorption or diffusion of therapeutic species into the wound tissue. In situations where the flow rate is too high or the fluid removal rate is too low, systemic absorption of therapeutic species may occur at an enhanced rate, increasing the risk of systemic toxicities and antibiotic resistance (as a consequence of exposing a broader range of microflora to the therapeutic species, e.g. acquired resistance). In a preferred embodiment, fluid may delivered to the tissue at a rate within a range of 5-16 mL/min, for example at a rate of about 13 mL/min. The fluid removal rate may be greater than the fluid delivery rate so as to ensure that a majority of the fluid is removed. It will be clear to one of ordinary skill in the art that other flow rate and suction rate ranges may be readily achievable utilizing this design.

In an effort to further determine the degree of absorption of local therapeutic species, a radiopaque (e.g. contrast agent) or fluorescent agent (e.g. indocyanine green) may be delivered in conjunction with the therapeutic agent to permit real-time visualization of the dispersion of the therapeutic agent. For example, an IV bag containing gentamicin could be mixed with indocyanine green and connected to the surgical device, bringing the IV bag fluid in contact with the wound. Via diffusion and absorption, the therapeutic species and the indocyanine green may be distributed spatially into the local tissues as described herein. Utilizing an appropriate imaging system (such as the SPY Elite System), the extent of fluorescent indocyanine green uptake can be visualized readily and quantified as a function of pixel intensity. Fluid flow and fluid removal flow rates can be adjusted as desired to achieve the desired local tissue concentrations.

Therapeutic Applications

It has been well-documented that a regimen of antibiotics before surgery and antibiotic lavage during surgery can lower the occurrence of SSI. Antibiotics may be systemically given to a patient prior to surgery (e.g. one hour before) in order to allow the antibiotic to distribute throughout the body and be present at the wound site during surgery. Currently, antibiotics may be chosen based on a determination of the bacteria or other skin flora typically present in the surgical site. For example, colorectal surgery patients may be treated with a different combination of antibiotics than cardiac surgery patients due to the differences in the flora commonly found in those sites. A doctor may for example use cefazolin, metronidazole, cefoxitin, cefotetan, ampicillin, sulbactam, ceftriaxone, ertapenem, or any combination thereof to prepare for a colorectal surgery while the preparation for a cardiac surgery may include cefazolin and/or cefuroxime. It may be beneficial to deliver antibiotics directly to the surgical site in addition to or instead of delivering the antibiotics systemically.

Any of the surgical devices described herein may be used to deliver one or more antibiotics directly to the wound. The antibiotic(s) may be delivered in the form of a fluid, a gas, a gel, powder, and/or a dissolvable solid. The surgical device may deliver the antibiotic(s) or other antimicrobial agents described herein to the wound such that the antibiotic(s) are dispersed onto the surfaces of the wound in order to inactivate the bacteria (and/or other microbes). The antibiotic(s) may be selected to target specific bacteria for inactivation and/or prevent incubation of specific bacteria in the wound (during and/or after surgery).

Any of the surgical devices described herein may be used to deliver one or more antimicrobial agent to the wound. The antimicrobial agent may comprise an antibacterial, an antifungal, an antiviral, an antiparasitic, or the like, or any combination thereof. The antimicrobial agent may comprise a plurality of antimicrobial agents. The antimicrobial agent may be used to target one or more microorganisms in the wound. The antimicrobial may comprise an antibacterial such as an antibiotic from any combination of the following antibiotic classes: ansamycins, carbapenems, beta-lactams, carbacephems, glycopeptides, lincosamides, licopeptides, monobactams, nitrofurans, penicillins, cephalosporins, macrolides, quinolones, oxazolidinones, fluoroquinolones, tetracyclines, polypeptides, or aminoglycosides.

The risk of developing a surgical site infection may be influenced by a range of factors attributable to the patient (e.g. nutritional status, immunocompromised status, blood sugar concentration, etc.) and invading organisms (e.g. virulence, concentration, classification, etc.). It is anticipated that the bacteria colonizing a surgical site may differ significantly from patient to patient. As an example, the surgical site may be a patient's abdomen or GI tract during a colorectal surgery. Different patients' GI tracts may differ significantly from one another. In many cases the choice of antibiotic (and/or whether to treat a patient prophylactically) may be based on general knowledge of the physician regarding the cause and/or likelihood of infection rather than on test results analyzing the patient microbiome and/or susceptibility to treatment. Such tests are rarely performed prior to symptoms of infection and may be beneficial if performed earlier so as to inform prophylactic treatment of patients and possibly prevent infections. In order to increase the effectiveness of a local therapeutic approach and choose the optimal therapeutic agent(s) for reducing the contamination of the surgical site and ultimately reducing surgical site infections, it may be desirable to determine which bacterial species colonize each patient's GI tract. Identification of the colonizing bacterial species may inform the choice of therapeutic regimen to target the bacteria most-likely to cause surgical site infection. The targeted therapeutic regimen maybe delivered systemically or locally to the surgical site as described herein. Selection of a therapeutic regimen following identification of the bacterial species may reduce patient exposure to excessive antimicrobials and/or reduce the risk of negative side-effects due to antibiotic overexposure.

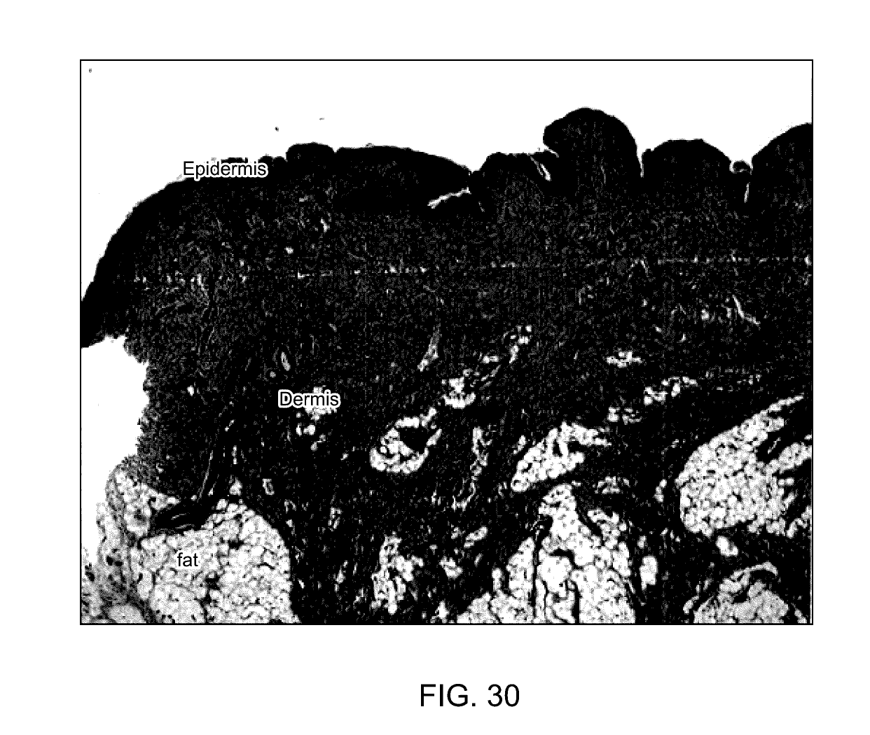

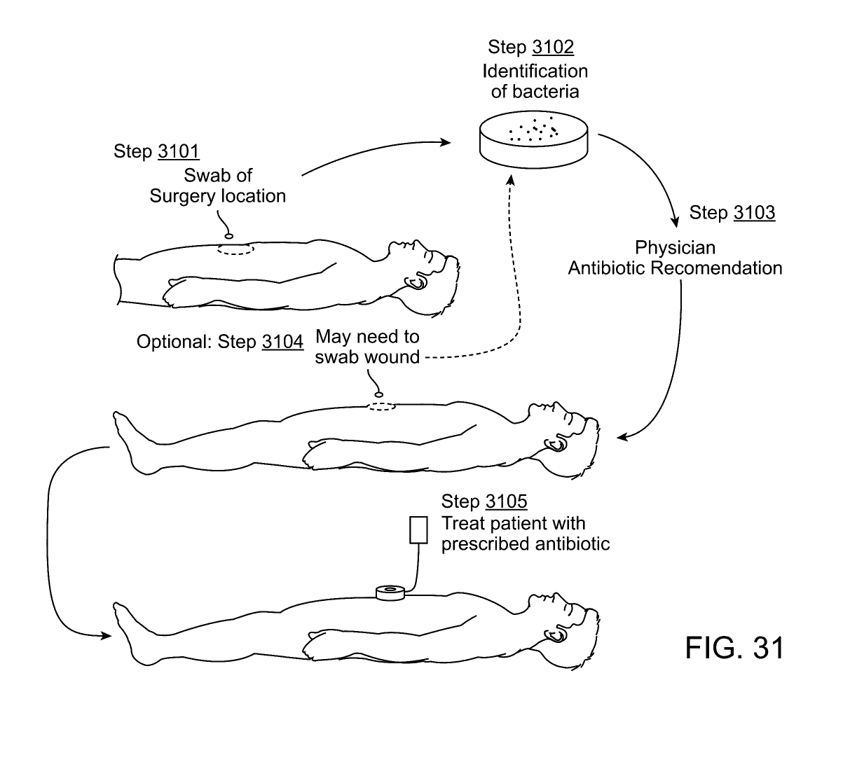

Methods of prophylactically treating a patient to prevent or reduce the risk of developing surgical site infections may comprise one or more steps such as collection of a sample, analysis of the sample, identification of the microbiotic make-up of the sample, determination of the risk of developing a surgical site infection, determination of a targeted treatment regimen, and/or implementation of the targeted treatment regimen before any symptoms of infection are detected (i.e. prophylactically). Exemplary methods are shown in FIGS. 31-32. It will be understood by one of ordinary skill in the art that any of the steps may comprise any of the techniques to perform the step described herein.

FIG. 31 shows a flowchart of a method for delivering a therapeutic agent to a surgical site using a surgical device. The surgical device may be any of the surgical devices described herein. Operative or post-operative treatments can be tailored to specific organisms found in the surgical wound, rather than the broad range of organisms that colonize each individual patient. This would generally be considered an on-demand point of care (POC) approach.

At Step 3101, a swab of the surgical site may be provided. During surgery, a culture swab of the surgical incision can be obtained and sent to the hospital's microbiology lab for analysis.

At Step 3102, the swab may be analyzed and the bacterial contents of the sample may be identified as described herein.

At Step 3103, the identification results may be provided to the physician and an antibiotic regimen recommendation may be made. Because bacterial wound contamination is a demonstrated risk factor for the development of surgical site infection, positive culture results may suggest the need for therapeutic intervention to reduce this risk. The therapeutic intervention selected may be tailored to combat the type of contamination identified in the surgical incision.

At Step 3104, the wound may optionally be swabbed after surgery. The wound may be swabbed in addition to or instead of taking a swab during surgery. Steps 3102 and 3103 may be repeated to identify the bacteria in the wound after surgery. When swabs have been take both during and after surgery, the identification results may be compared in order to determine if the bacterial population has changed and better inform post-surgical treatment decisions.

At Step 3105, the patient may be treated with the antibiotic regimen identified in Step 3103. For example, if E. coli were found to be present in the incision, intravenous gentamicin could be administered at 100 mg twice daily post-surgically in order to reduce the risk of developing surgical site infection. Alternatively or in combination, topical gentamicin could be delivered intraoperatively to directly counteract the activity and growth of the E. coli present in the surgical wound.

Any of the surgical devices described herein may be used to deliver the antibiotic regimen to the wound. A system or kit may be provided comprising the surgical device incorporating fluid delivery (and/or removal) with a culture swab and media (e.g. E-Swab by Copan, Murrietta, Calif.). Throughout the duration of the surgical procedure, the surgical device may be used with or without fluid delivery functionality enabled. Furthermore, the irrigation fluid may or may not comprise therapeutic agents such as antibiotics. At any point during the procedure, and preferably prior to closure of the incision, the culture swab can be used to sample the bacteria or other organisms residing within the incision, which can be sent to the hospital microbiology lab for identification of organisms present. Upon determination of the type and/or number of organisms present (or various other factors as described herein), the irrigation fluid may be replaced with a more appropriate irrigation fluid having properties known to inhibit the growth of the identified organisms(s). For example, if E. coli was found to be contaminating the wound, gentamicin could be used as a therapeutic agent delivered to the wound tissue through the fluid delivery function of the surgical device.

Although the steps above show a method of delivering a therapeutic agent to a surgical site in accordance with embodiments, a person of ordinary skill in the art will recognize many variations based on the disclosure provided herein. The steps may be completed in a different order. Steps may be added or deleted. Some of the steps may comprise sub-steps. Many of the steps may be repeated to achieve the desired therapeutic regimen.

FIG. 32 shows a flowchart of a method for identifying the bacterial species present at the surgical site in order to direct therapy.

At Step 3201, a stool sample may be provided by the patient. The stool sample may be provided pre-operatively or post-operatively.

At Step 3202, the stool sample may be cultured or otherwise prepared as described herein for sample identification.

At Step 3203, one or more pathogenic organism (e.g. fungi, bacteria, virus, etc.) may be identified using any of the methods described herein. The sample may for example be tested to identify the type and number (colony forming units) of aerobic and anaerobic bacteria and other organisms colonizing the patient's GI tract.

At Step 3204, one or more antimicrobial agents may be selected based on the identification of the pathogenic organisms in the sample. For example, the culture results might indicate that a patient is colonized with S. aureus and E. coli. Thus, an antibiotic regimen may be selected which targets both S. aureus and E. coli. Alternatively or in combination, the antibiotic regimen may be selected based on other factors besides the strict presence or absence of a bacterial strain. The method may further comprise an optional step 3204a in order to compare the identified organism with other factors which may affect the risk of developing an infection or otherwise inform a treatment strategy. For example, data published by the CDC reporting the frequency with which certain organisms are cultured from surgical site infections, stratified by procedure type, may be used to determine which bacterial species identified in the sample is most likely to cause an infection in a given procedure type. In colon surgery, if 67% of surgical site infection are found to be colonized with E. coli, then E. coli may be considered to be a virulent strain and its presence in the sample may indicate that the patient is at elevated risk of developing a surgical site infection. Antimicrobial agent(s) may be selected so as to target a broad range of organisms cultured from the stool sample or a selected subset of virulent strains (such as E. coli in colon surgery patients). Selection of an antimicrobial agent to target the virulent strain(s) may reduce the risk of developing antimicrobial resistance or other side-effects of antimicrobial agent use. As an illustrating example, gentamicin might be selected to target the E. coli colonizing a patient's GI tract, the E. coli have been flagged as a virulent strain because the patient is scheduled for colon surgery.

At Step 3205, the selected antimicrobial agent may be delivered to the wound via the surgical device as described herein in order to prophylactically treat the tissue and reduce the risk of wound contamination during surgery. Alternatively or in combination, the selected antimicrobial agent may be delivered to the wound after surgery.

Although the steps above show a method of prophylactically delivering a therapeutic agent to a surgical site in accordance with embodiments, a person of ordinary skill in the art will recognize many variations based on the disclosure provided herein. The steps may be completed in a different order. Steps may be added or deleted. Some of the steps may comprise sub-steps. Many of the steps may be repeated to achieve the desired therapeutic regimen.

It will be clear to one skilled in the art that the methods described herein may permit a range of permutations. For example, in Step 3201, instead of a stool sample, a skin or nasal swab, or any of the sample types described herein, could be taken. At Step 3204, the determination of a treatment method may include comparing the identified sample microbes with the antibiogram of the hospital. The antibiogram may indicate microbial patterns within that hospital which may be informative as hospitals often have distinct patterns which may differ from the patterns seen in larger country-wide studies as in the case of the CDC data. The antibiogram may detail the activity (as measured in minimum inhibitory concentrations) of certain antimicrobial agents against common bacterial species. Information from the CDC, sample analysis, and other sources as described herein may be combined with information from the antibiogram in order to select an appropriate antimicrobial having a lower (or the lowest) minimum inhibitory concentration against a specific organism.

A patient sample may for example comprise a skin or wound swab from the surgical site. The sample may comprise a biopsy of the wound or tissue near the surgical site. The patient sample may be collected using any sample collection method described herein or known to one of ordinary skill in the art with reference to the surgical site of interest. The patient sample may be collected prior to, during, and/or after surgery. While reference is made to a single patient sample being acquired and analyzed, it will be understood that the patient sample may comprise a plurality of patient samples. The plurality of patient samples may be collected over a period of time. Alternatively or in combination, the plurality of patient samples may be collected from a plurality of sample locations or with a plurality of sample collection methods.

In the exemplary example of colorectal surgery, the patient sample may comprise one or more or a stool sample, a mucosal biopsy, a mucosal brushing, an aspirate, a colonoscopy fluid aspirate, a rectal swab, a nasal swab, a wound swab, a bowel prep collection, a sample from a color-changing bandage (as described herein), or any combination thereof.

Stool samples have been the traditional sampling method used for microbiome research, however there may be significant differences between the microbiota found in stool compared to other locations in the patient's GI tract. Some sample types, for example stool sample or rectal swabs (i.e. luminal samples collected after mechanical mixing throughout the colon), largely contain transient bacteria. While these bacteria contribute to the microbiome's dynamic nature, there may be species which are more persistent deep within the mucosal tissue of the gut which may be more relevant in the surgical setting as typically the contents of the colon are emptied (along with the transient bacteria residing the lumen) at the time of surgery. Many gastrointestinal surgeries are done after mechanical or oral bowel preparations that empty the colon for example. Sampling methods such as taking a mucosal biopsy or mucosal brushing may provide samples comprising more persistent species. A mucosal biopsy or brushing sample may be taken prior to surgery and the microbiome of the patient may be analyzed as described herein. A biopsy sample may for example be obtained during a pre-surgical screening procedure such as a diagnostic colonoscopy which may occur one to two weeks prior to the date of surgery. Alternatively or in combination, a biopsy sample may be obtained on the date of surgery. In some instances, a stool sample may be able provide information on mucosal microbiota if collected under the right conditions. The stool microbiota may be very similar to mucosal microbiota if the stool sample is taken after the patient has undergone bowel-preparation. Following bowel-preparation and prior to surgery, a stool sample may be obtained from the patient and analyzed in order to determine if (and what) therapeutic intervention may be desired prophylactically (e.g. during or after surgery) as described herein. Such a sample may be beneficial as it may be easily obtained and may not require a healthcare provider or additional procedures to obtain.

In contrast to obtaining a pre-operative sample, the clinical intervention selected could be tailored to combat the type of bacterial contamination identified from sampling within the surgical incision itself, for example by swabbing the wound or obtaining a biopsy or brushing during surgery. This would generally be considered an on-demand point of care (POC) approach. Upon characterization of the microbiome within the surgical site, a standard intra-operative irrigation fluid could be replaced with a more appropriate therapeutic agent having properties known to inhibit the growth of the identified organism(s). This could be delivered through the surgical device described herein, an intravenous line, topical administration, or any delivery method known to one of ordinary skill in the art.

Any of the methods described herein may be adapted for post-surgical therapeutic intervention. The wound may be monitored for bacterial growth and therapeutic agents may be provided to the patient if an infection is suspected or identified. The wound may be monitored such that bacterial growth is identified prior to the development of clinical symptoms of infection. For example, a swab of the wound may be taken one or more times after treatment and tested for bacterial growth as described herein. Alternatively or in combination, a new “intelligent” color-changing bandage, such as that developed recently at the University of Bath, could serve as an early post-surgical detection system for infection. The bandage may comprise tiny dye-containing capsules that respond to contact with populations of pathogenic bacteria. The outer layer of the capsules may be designed to mimic certain aspects of a cell membrane such that when a pathogenic microbe puncture the capsules (as they would eukaryotic cells) the capsules release a visible dye. A healthcare provided may monitor the color of the bandage in order to identify if bacterial growth is occurring. Samples may be collected from the bandage in order to determine which potentially harmful bacterial species reside in the patient. The species identified may then be targeted for therapeutic intervention as described herein. For example, if E. coli were found to be present within the bandaged incision, intravenous gentamicin could be administered at 100 mg twice daily post-surgically to reduce the risk of developing a full-blown surgical site infection. The bandages may continue to be used and monitored during treatment in order to monitor the efficacy of therapy as well as to survey for emergence of other (resistant) bacterial species which may require additional intervention.

In order to determine the risk of developing a surgical site infection and/or to inform treatment regimen decisions, the patient sample collected may be analyzed to provide information about the patient's microbiome. This may entail, at a general level, assessing the patient sample for information about pathogen abundance and resistance in the sample. The sample may be collected using any of the sample collection methods described herein or known to one of ordinary skill in the art. The sample may be analyzed using culture-dependent methods, culture-independent methods, or any combination thereof.

Culture-dependent methods may comprise plating on selective media, biochemical identification, gram staining, phenotypic characterization, polymerase chain reaction (PCR), real-time PCR, quantitative real-time PCR (qPCR), reverse transcription PCR (RT-PCR) mass spectrometry, matrix-assisted laser desorption-time of flight (MALDI-TOF) mass spectrometry, DNA sequencing, ELISA, reverse genome probing, any other culture-based method known to one of ordinary skill in the art, or any combination thereof.

Culture-independent methods may comprise, 5.8S rDNA sequencing, 18S rDNA sequencing, 28S rDNA sequencing, 16S rDNA phylogenetic surveys, 16S rRNA next generation sequencing, 5S rRNA sequencing, 23S rRNA sequencing, PCR, qPCR, RT-PCR 454-pyrosequencing, bTEFAP pyrosequencing, high-throughput sequencing, deep sequencing, next generation sequencing, fungal ITS amplicon analysis, mass spectrometry, MALDI-TOF mass spectrometry, mass spectrometry in selected reaction monitoring mode (MS-SRM), high performance electrospray ionization mass spectrometry (ESI-MS), phage-based detection, polarization anisotropy diagnostics, chromatography, any other culture-independent method known to one of ordinary skill in the art or any combination thereof.

After a microbiome sample is acquired, the sample may be cultured for further analysis or the bacterial sample may be processed for analysis without culture. In some instances, DNA or RNA may be extracted from the sample using any extraction technique known to one of ordinary skill in the art. Many different approaches could be used to characterize or sequence the prokaryotic/viral genomes within the sample, identifying the type and number (colony forming units per gram) of colonized microorganisms, as well as whether they carry any known virulent or antibiotic resistant genes. Some of these approaches are outlined herein. It will be understood that the sample may be characterized using any technique known to one of ordinary skill in the art.

Whole-cell mass spectrometry (e.g. MALDI-TOF, MS-SRM, etc.) may for example be used for microbial identification and susceptibility testing. Such techniques may provide relatively quick result turnaround, such as within about 24 hours or less, which may allow a healthcare provider to identify components of the microbiome prior to or shortly after performing a surgical procedure. Intact bacterial or bacterial particles may be purified from the patient sample and fragmented by enzymatic digestion into peptides. The peptides may be isolated and subsequently resolved using chromatography separation and electrospray triple quadrupole (ESI-QqQ) MS or other mass spectrometry techniques. In ESI-QqQ, the three most intense pair of precursors for each peptide may be filtered based on the mass-to-charge ratio. Proteotypic peptides may track features specific to one or more bacterial species in the sample. In the example of S. aureus characterization, the peptides may be used to identify the bacterium down to the species level, detect resistance mechanisms (for example penicillin binding proteins (PBP) 2a and/or PBP2c characteristic of methicillin-resistant S. aureus (MRSA)), and/or detect virulence mechanisms (such as toxin production like panton-valentine leucocidin (PVL) and/or toxic shock syndrome toxin (TSST-1)).

Alternatively or in combination, next-generation sequencing techniques may be used to analyze DNA and/or RNA isolated from the patient sample. Sequencing techniques may be done on DNA or RNA isolated directly from the patient sample or which has been amplified prior to sequencing using any technique known to one of ordinary skill in the art. Techniques such as 16S rRNA next generation sequencing may enable analysis of the entire microbial community within the sample. Deep sequencing methods may be high-throughput and provide information on the individual sequences for millions of DNA molecules, thereby enabling each to be classified independently. 16S rRNA gene sequencing may be used to identify types of bacteria and archaea as the 16S ribosomal RNA gene is ubiquitous. Sequences from a 16S rRNA-targeted amplicon read may be used for genus and/or species level taxonomic identification.

Alternatively or in combination, techniques such as polarization anisotropy. Genetic material from the patient sample may be collected and loaded onto a plastic cube. DNA probes may be loaded into the cube to detect genetic sequences characteristic of a disease-causing bacterium. Reporter probes comprising fluorescent tags may be added to the cubes to track to the DNA probes (and/or target bacterial RNA sequences when RNA is of interest) to generate distinct polarizing light signals. The light signals may be measured, digitized, and transmitted to a processor for further analysis, for example the processor described herein. Such a method could be completed quickly (e.g. in two hours or less) making it easily implementable for bacterial identification prior to, during, or after a surgical procedure. For example, a typical colorectal surgery may take about 4 hours, thus a sample taken at the start of the procedure may return results which may inform the choice of intra-operative or post-operative prophylactic therapeutic regimen for the patient as described herein.

Many different approaches could be used to characterize or sequence the microbiota or a patient sample. A few approaches are outlined herein. It will be understood by one of ordinary skill in the art that any analysis method may be used to generate information about the composition of the microbial species in the sample. Data may be generated in order to identify the type of microbes present in the sample, the relative abundance of the microbes in the sample, the virulence of the microbes (including whether they carry any known pathogenic mutations or genes), the antibiotic susceptibility of the microbes (including whether they carry any known antibiotic resistance mutations or genes), or any other identifying characteristic known to one of skill in the art, or any combination thereof.

The treatment regimen may be determined based on characterization and/or sequence results of the microbiota or patient sample as described herein. For example, the composition of a patient's microbiome may be determined using any of the methods described herein. The composition may comprise a list of bacterial species and their relative abundance in the patient sample. The most abundant species (or plurality of species) may be selected as the target microorganism(s) and one or more antibacterial agents known to be effective on the target microorganism(s) may be selected as the therapeutic regimen. Alternatively or in combination, the list of bacterial species may be compared to one or more datasets (like that produced by the CDC or other risk database described herein) in order to determine which species (or plurality of species) may be most likely to cause a surgical site infection and the species identified may be targeted. Alternatively or in combination, the virulence of the microorganisms in the microbiome may be determined in order to inform the choice of target microorganism. More virulent species (e.g. those with antibiotic resistance or virulence genes) may be targeted instead of a more abundant species as likely to cause surgical site infection. The choice of therapeutic agent may be based on common therapeutic susceptibilities of the target microorganism and/or based on an antibiogram of the hospital or other susceptibility database as described herein. Alternatively or in combination, the choice of therapeutic agent may be based on the virulence of the target species, particular when the target microorganism has one or more antibiotic resistances which may suggest non-standard therapeutic regimen. Alternatively or in combination, the choice of therapeutic agent may be made following susceptibility testing with the antibiotics themselves as known to one of ordinary skill in the art.

The treatment regimen may be tailored to treat more than one bacterial species or identified risk factor for the development of a surgical site infection. The treatment regimen may comprise one or more of the following antimicrobial techniques: antibiotics or other antimicrobial agents as described herein, phage therapy, peptide phosphorodiamidate morpholino oligomers (PPMOs), antimicrobial peptides, osmoregulation, probiotics, bacteriocin, bacteriocin-producing probiotics, gene therapy (CRISPR, electroporation, microinjection, lipofection, polyplexes, lipoplexes, viral delivery, etc.), microbiome deletion with macrophage cocktail, or any other antimicrobial technique known to one of ordinary skill in the art.

Neural Networking

The methods and systems described herein may further utilize advanced computing techniques such as machine learning and/or neural networking in order to better understand the nature of surgical site infections and better predict surgical outcomes.

Identification of the sample's microbial makeup may be used to inform subsequent treatment decisions. As described herein, in a simplistic example, the presence or absence of a particular bacterial species (or combination of species) may strongly correlate with the development of a surgical site infection. Given the identification of such a species in the sample, it may be desirable to prophylactically provide therapeutics (e.g. antibiotics) to the patient prior to, during, or after surgery. In many cases, many factors may affect a patient's risk of developing a surgical site infection. The way these factors contribute to the risk of developing a surgical site infection or how they interact within each other may be poorly understood or often completely unknown. Because little may be known about how the microbiome operates macroscopically, it may be difficult to correctly predict surgical outcomes based solely on the knowledge of abundance and resistance. Bacteria may neither appear abundant nor resistant but their ability to synergize and exploit a stressed tissue environment can accelerate their proliferation or mutation.

An artificial neural network or other machine learning technique may leverage the multifactorial computing capability of a processor in order to assess the risk of developing a surgical site infection and/or inform treatment decisions for a variety of input factors. The neural network may further provide information about the relative contribution of the input factors to the desired output, which may be beneficial in furthering understanding about surgical site infections. The neural network may be configured with instructions to analyze a large dataset of information (e.g. factors) with the potential to identify microbial patterns of certain environments and correlate them to surgical outcomes. The network may act as a database, storing individual patients' microbiome characterization data. Alternatively or in combination, the network may act as a branch of machine learning, using a set of algorithms (e.g. instructions) to attempt to model high-level abstractions in the input data for example by using a deep graph with multiple processing layers to allow for a level of information about the microbiome not yet harnessed or understood with currently available methods.

The neural network may comprise a radial basis function (RBF) network, a Kohonen self-organizing network, a leaning vector quantization, a recurrent neural network, a modular neural network, a physical neural network, a holographic associative memory, an instantaneously trained network, a spiking neural network, a dynamic neural network, a cascading neural network, a neuro-fuzzy network, a compositional pattern-producing network, a one-shot associative memory, a hierarchical temporal memory, or any other neural network known to one of ordinary skill in the art. The neural network may be configured for unsupervised learning, continued learning, fixed learning, or deep learning.

An artificial neural network works to mathematically mimic human neural architecture in order to provide the network with “learning” and “generalization” capabilities (and as such may be considered part of the field of artificial intelligence). An artificial neural network may be useful in modeling the causes and/or determining how to treat surgical site infections as they can model highly non-linear systems in which the relationship between input variables is unknown and/or complex.

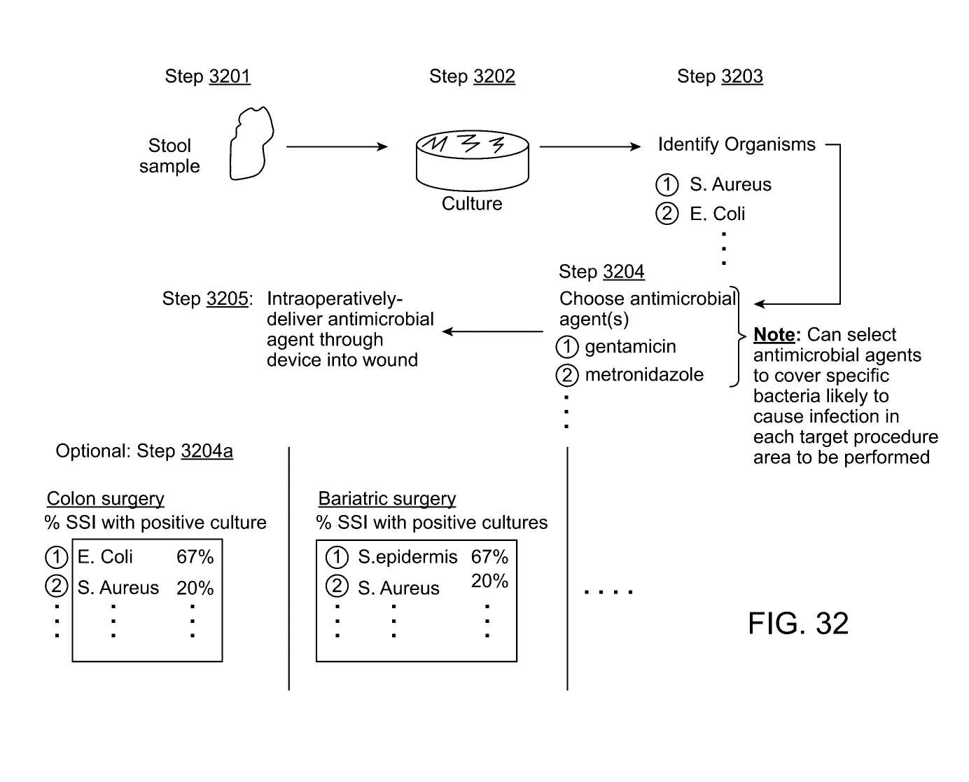

FIG. 33 shows a schematic diagram of an exemplary artificial neural network which may be used to provide a desired output. The network may comprise a series of neurons (also referred to as nodes) which are organized into a layered structure. Each neuron in a layer may be connected to one or more of the neurons in the next layer by a synapse. The synapse may be configured to apply a weight to the input variable prior to delivery to the next layer. The weight may indicate the strength of the connection between the two neurons connected by the synapse. The neural network may be structured so as to comprise an “input” layer, one or more “hidden” layers, and an “output” layer. The number of neurons in each layer and the number of layers in the neural network may depend on the complexity of the system being studied and/or the question being asked (e.g. the desired output). Neurons in the input layer may receive data from a user in the form of an input. The user may for example input the data via a user interface as described herein. The input data may be transferred to the first hidden layer through a weighted synapse. Data in the hidden layer may be mathematically processed and the results may be transferred to the next layer (e.g. another hidden layer or the output layer). This may be repeated for each of the connections between layers until the output layer is reached. Although three layers are shown here as an illustrative example, it will be understood by one of ordinary skill in the art that any number of layers may be provided. Adding additional layers may provide a more refined analysis, which may in turn provide a more reliable output.

At the input layer, a user may input values for any number of variables. For example, the input layer may comprise three neurons in which data for species quantity, virulence rank, and prophylaxis used may be input. Each synapse may apply a weight to the data from the input neuron. For example, the input of neuron “a” may comprise a species quantity. The species quantity may be weighted by the synapse using Equation 9.

Inputs_{species quantity}*W(a)=X(a) (9)

where W(a) is the weight applied to the input value and X(a) is the weighted synapse output. The weighted synapse output X(a) may then be sent to hidden layer “a”. The weighted synapse outputs from each of the synapses connected to hidden layer “a” (e.g. X(a) from synapse a, X(b) from synapse b, and X(c) from synapse c) may be combined to become the hidden layer neuron input Z(1) for hidden layer 1 using the Equation 10.

X(a)+X(b)+X(c)+0(1)=Z(1) (10)

The synapse may further add a “bias” term θ(1) when calculating the weighted sum Z(1) if desired. The weighted sum Z(1) may then be transformed by a mathematical transfer function and transferred to the next layer (in this example the output layer). The transfer function may for example be a sigmoid transfer function shown in Equation 11

$$\frac{1}{1 + {e}^{Z ( 1 )}} = A ( 1 ) \qquad ( 11 )$$

where A(1) is the result of the transfer function which may then be transferred to the next layer, in this case the output layer. At the output layer, the transfer function results of the hidden layer, for example A(1), A(2), and A(3), may be combined to reach the predicted outcome ŷ, for example the risk of developing a surgical site infection, as the output. Although only one output node is depicted, it will be understood by one of ordinary skill in the art that any output desired may be determined.

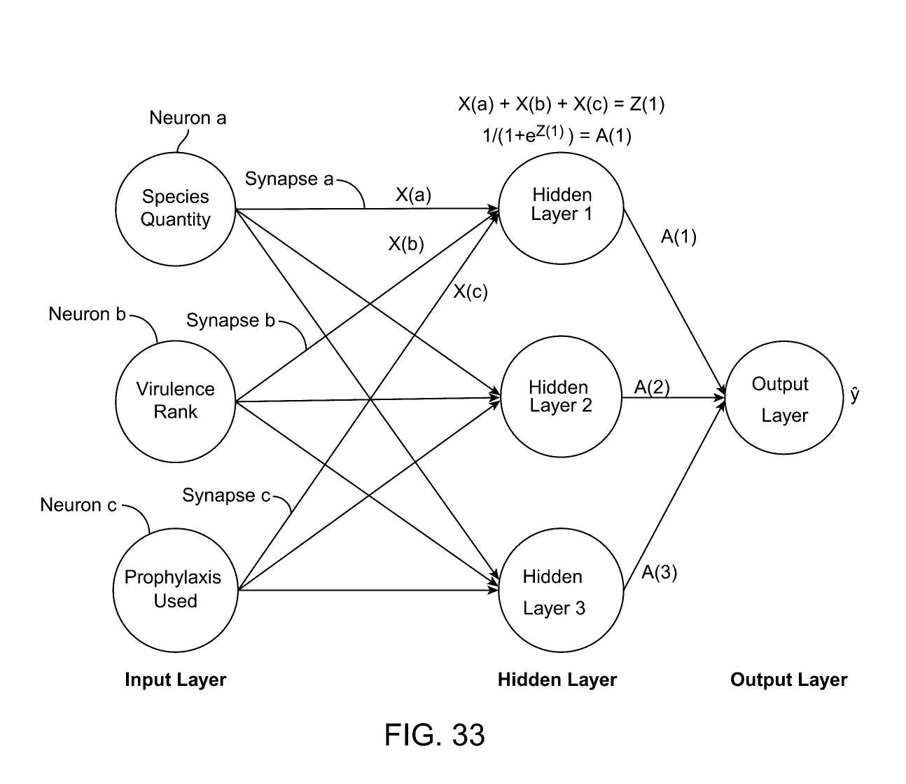

FIG. 34 shows exemplary matrices which may be used to structure and train the neural network. The neural network may be configured to receive a vector (e.g. matrix) with a number of inputs (for example two per patient as shown) and provide a vector with a number of outputs (for example one—surgical site infection—as shown). The neural network may be trained using a training data set in which the patient data includes both the desired input factors as well as known outcomes (e.g. development of surgical site infection). The network may “learn” from the exemplary training data set. While the training data set shown comprises example data from 4 patients, it will be understood that the number of examples in the training data set may be much higher, for example on the order of hundreds or thousands of patient examples. Each patient example in the training data set may comprise an input matrix and an output matrix. The input matrix may for example comprise data representing the abundance of an identified bacterial species (e.g. corresponding to neuron a in FIG. 33) and the virulence of the identified bacterial species (e.g. corresponding to neuron b in FIG. 33). The objective of the training process may be to approximate the function ƒ between the input matrix and the output matrix of a patient in order to later use the network to predict output matrix values with high accuracy. This may be achieved by iteratively changing the values of the synapse weights W according to a training algorithm. The training algorithm may for example comprise a cost function used to compare the output generated by the neural network to the known value of the output each patient. The training data set may be used which comprises data from multiple patients with known outcomes, for example whether or not the patients developed a surgical site infection. A comparison of the calculated outcome with the actual output may be done using the delta function shown in equation 12.

\hat{y}*(1−ŷ)*(y)=Δ (12)

The difference Δ between the calculated outcome ŷ and the actual patient outcome y may then be back-propogated into the hidden layer(s) and used to change the weights applied by the synapses. The network may run through multiple iterations (e.g. hundreds or thousands) until the Δ value approaches or reaches 0 and the cost function is minimized, thereby training the network to generate an output ŷ approximately equal to the known outcome y of the patient.

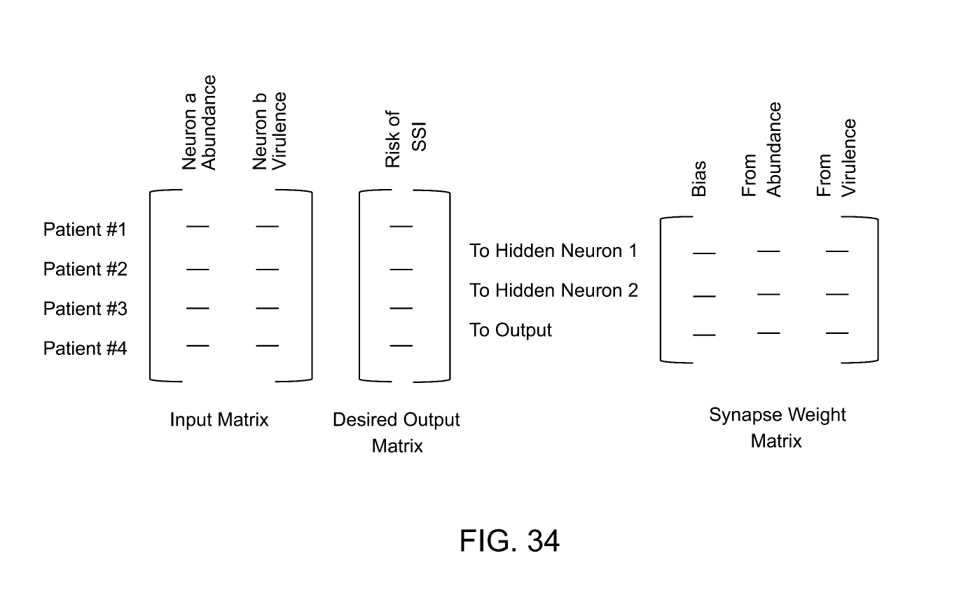

FIG. 35 shows a graphical representation of a possible relationship between input variables and the desired output which was be obtained using the network trained as in FIG. 34 with a training dataset comprising sham patient data. The prototype network may be configured to return the synaptic weights that create an output approximately equal to the desired output. The weights may indicate how much their respective input nodes contribute to the output of whether a patient received an infection or not. Inputs with high associated weights may be targeted for prophylaxis as described herein. When there are enough examples in the network (at least 10 times as many examples as degrees of freedom), the weights can be used on their own, without a desired output matrix, to determine a patient's output (e.g. predict the output based on training with prior patient data). The network may be configured to report out binary outputs (e.g. “no risk of infection” or “will be infected”). The network may be configured to report out non-binary outputs (e.g. percent risk of infection).

Once trained, the network may be configured with instructions to determine the most likely source of infection in a patient and/or targeted treatment regimen to treat the source of infection. For example, a user may enter an patient's enteric bacterial species identified with associated quantity (abundance) and virulence, as well as that individual's surgical outcome (surgical site infection or no surgical site infection). Other possible nodes of information that may be useful input include hematocrit, ferritin levels, patient body mass index, the type/strength of intra-operative prophylaxis that was used, the classification of the wound, or any other input described herein or known to one of ordinary skill in the art. The network could be trained to calculate which input(s) most significantly contribute(s) to an output of infection based on past examples (within the network itself) of patients who had infection. Prophylaxis could then be tailored towards specific inputs identified. Alternatively or in combination, the program could calculate the percent likelihood that a person with inputs “a, b, and c” will acquire an infection. This information could advise about the level of prophylaxis optimal for a particular patient, saving resources and money for patients that don't require a strong course of action. Alternatively or in combination, the network could be configured with instructions to detect patterns in patients who did not acquire a surgical site infection, thereby potentially identifying prophylactic microorganisms that may be used to help revert the surgical environment to symbiosis during the re-proliferation period (post-bowel preparation and post-surgical stress). Once these microorganisms have been identified, they may inform toward probiotic prophylaxis in place of antibiotic prophylaxis.

Inputs may include information about the makeup of the patient's microbiome risk factors for developing surgical site infections, net rank of sources of surgical site infection given a particular surgery or surgical site, patient susceptibility factors, hospital susceptibility factors, or any combination thereof, or any other factor described herein or known to one of ordinary skill in the art. The artificial neural network may process the inputs as described herein to generate an output. The output may for example comprise a calculated risk of developing a surgical site infection, a suggested treatment regimen to use, a ranking of inputs in order of their relative contribution to the risk determination, or any other output described herein or desired by one of ordinary skill in the art in order to inform treatment of a patient or understanding of surgical site infections.

Inputs to the neural network may comprise bacterial count, relative bacterial abundance, bacterial virulence (for example the presence/absence of virulence genes or toxin production), bacterial rank in frequency of causing surgical site infection in a population, patient demographics, patient risk factors, patient allergies, patient allergies to therapeutic agents, patient vitals, patient body mass index (BMI), surgical procedural details, persistant microbiome species, transient microbiome species, antibiotic susceptibilty, hospital antibiogram, environment-specific antibiotic susceptibility, any other input or factor described herein, or any combination thereof, or any other factor known to one of ordinary skill in the art.

Outputs generated by the neural network may comprise any of the factors described herein or known to one of ordinary skill in the art. The output may comprise any of the factors described herein as possible inputs, for example the output may include information about how each input contributes to the risk of developing surgical site infection, the provide therapeutic regimen, or any other output of interest to one of ordinary skill in the art. Outputs generated by the neural network may comprise bacterial count, relative bacterial abundance, bacterial virulence (for example the presence/absence of virulence genes or toxin production), bacterial rank in frequency of causing surgical site infection in a population, patient demographics, patient risk factors, patient vitals, patient body mass index (BMI), surgical procedural details, persistant microbiome species, transient microbiome species, antibiotic susceptibilty, hospital antibiogram, environment-specific antibiotic susceptibility, any other output (or input or other factor) described herein, or any combination thereof, or any other factor known to one of ordinary skill in the art.

The input may comprise raw lab results or the lab results may first be screened to select the most relevant results prior to input. For example, identification of the abundance of bacterial species in a sample may be compared to a list of known players responsible for surgical site infections at the surgical site (e.g. the CDC report described herein). The raw data may for example be compared to a database comprising information about surgical site risk factors. In some instances, the most abundant bacterial species may not be the most likely cause of surgical site infections at the surgical site of interest. In that case, the most likely infectious agent(s) may be selected as an input over the most abundant infectious agent(s). Alternatively or in combination, the most abundant bacterial species may not be the most virulent species present prior to or during surgery. Comparison of the bacterial species identified with their relative virulence may point to species other than the most abundant species as a likely cause of (or contributor to) infection. Optionally, comparison of the identified organisms with a antimicrobial susceptibility database (for example comprising the hospital antibiogram) may point to treatment regimen (dose, timing, etc.) with improved targeting of the identified organisms.

A number of factors have been correlated with increased risk of surgical site infection including microbial density (e.g. greater than 10⁴ colony forming units when cultured), microbial species (with species such as S. aureus causing about 16%, Enterococcus spp. 14%, and E. coli 12% of abdominal surgical site infections), microbial synergy (e.g. oxygen consumption by aerobic bacteria inducing tissue hypoxia and allowing anaerobic bacterial growth), the host immune response (e.g. macrophage activity, white cell count, etc.), the quality of the tissue (e.g. hypoxia, perfusion), drug resistance, and/or virulence factors of the microbial species (e.g. toxicity, aggressiveness, replication, adherence/attachment to tissue, antigenic variation, immunologic reactions, etc.). Any of the factors described herein may be used as inputs or outputs in the neural network.

Factors which may correlate with surgical site infection may comprise one or more characteristic of the wound (e.g if the wound comprises non-viable tissue and/or foreign material), characteristics of the operation (e.g. surgery performed, techniques used, surgical suite temperature, presence of exogenous infectious species in the surgical suite, etc.), systemic patient factors (e.g. malnutrition, obesity, poor tissue perfusion, etc.), composition of the microbiome (e.g. abundance and/or virulence of microbial species), or any combination thereof.

Surgical factors may include surgical classification, skin preparation, site of surgery, duration of surgery, complexity of surgery, presence of suture or other foreign body material, quality of suturing, pre-existing local and/or systemic infection, prophylactic antibiotic use, haematoma, mechanical stress on the wound, duration of surgery, incision size, extent of blood loss, pre-operative shaving, type of skin closure (e.g. staple, suture, or other technique), surgical instruments used, experience or competency of the surgeon or other healthcare provider, intra-operative complications such as ureter injury or electrocautery injury, or any combination thereof.

Anesthetic factors may include the extent of tissue perfusion, normovolaemia or hypovolaemia, per-operative body temperature, intra-operative body temperature, concentrations of inspired oxygen, pain, blood transfusion, or any combination thereof.

Patient-related factors may include diabetes, smoking, poor nutrition, alcoholism, weight, obesity, chronic renal failure, jaundice, age, advanced age, poor physical condition, medication, previous chemotherapy, previous radiotherapy, immunosuppression, or any combination thereof.

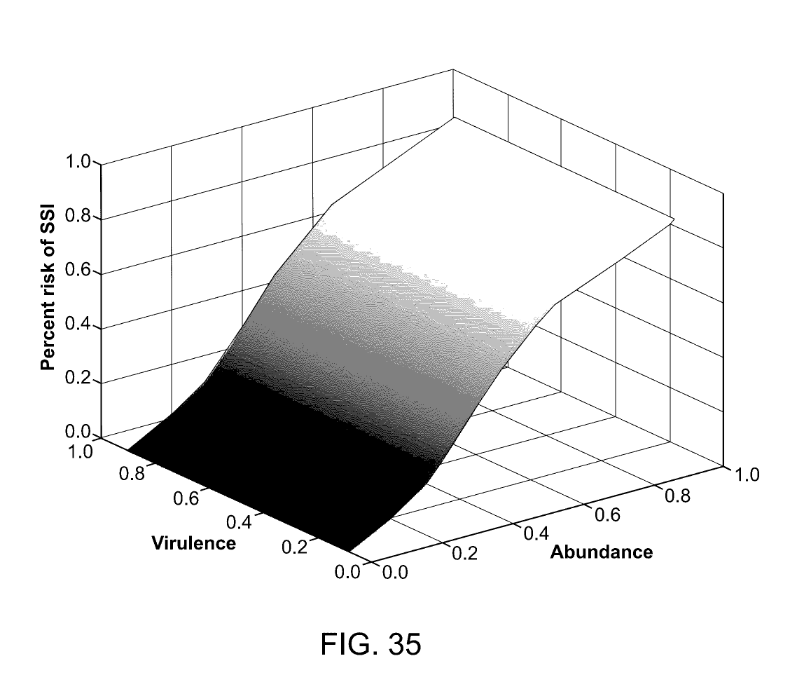

The present disclosure may provide computer control systems that can be programmed to implement methods of the disclosure. FIG. 36 shows a computer system 3601 that can be programmed or otherwise configured to analyze data comprising information about a patient's microbiome composition and determine whether (and/or how) prophylactic treatments should be administered to the patient. The computer system 3601 can regulate various aspects of any of the methods to determine the risk of infection and/or a prophylaxis treatment regimen of the present disclosure, such as, for example, running the neural network described herein in order to take a set of relevant inputs from a user (and/or database) and transform them into an output such as a risk of a patient developing a surgical site infection as described herein. The computer system 3601 can be an electronic device of a user or a computer system that is remotely located with respect to the electronic device. The electronic device can be a mobile electronic device.

The computer system 3601 may include a central processing unit (CPU, also “processor” and “computer processor” herein) 3605, which can be a single core or multi core processor, or a plurality of processors for parallel processing. The computer system 3601 also may include memory or memory location 3610 (e.g., random-access memory, read-only memory, flash memory), electronic storage unit 3615 (e.g., hard disk), communication interface 3620 (e.g., network adapter) for communicating with one or more other systems, and/or peripheral devices 3625, such as cache, other memory, data storage and/or electronic display adapters. The memory 3610, storage unit 3615, interface 3620 and/or peripheral devices 3625 may be in communication with the CPU 3605 through a communication bus (solid lines), such as a motherboard. The storage unit 3615 can be a data storage unit (or data repository) for storing data. The computer system 3601 can be operatively coupled to a computer network (“network”) 3630 with the aid of the communication interface 3620. The network 3630 can be the Internet, an internet and/or extranet, or an intranet and/or extranet that is in communication with the Internet. The network 3630 in some cases is a telecommunication and/or data network. The network 3630 can include one or more computer servers, which can enable distributed computing, such as cloud computing. The network 3630, in some cases with the aid of the computer system 3601, can implement a peer-to-peer network, which may enable devices coupled to the computer system 3601 to behave as a client or a server.

The CPU 3605 can execute a sequence of machine-readable instructions, which can be embodied in a program or software. The instructions may be stored in a memory location, such as the memory 3610. The instructions can be directed to the CPU 3605, which can subsequently program or otherwise configure the CPU 3605 to implement methods of the present disclosure. Examples of operations performed by the CPU 3605 can include fetch, decode, execute, and writeback.

The CPU 3605 can be part of a circuit, such as an integrated circuit. One or more other components of the system 3601 can be included in the circuit. In some cases, the circuit is an application specific integrated circuit (ASIC).

The storage unit 3615 can store files, such as drivers, libraries and saved programs. The storage unit 3615 can store user data, e.g., user preferences and user programs. The computer system 3601 in some cases can include one or more additional data storage units that are external to the computer system 3601, such as located on a remote server that is in communication with the computer system 3601 through an intranet or the Internet.

The computer system 3601 can communicate with one or more remote computer systems through the network 3630. For instance, the computer system 3601 can communicate with a remote computer system of a user (e.g., a smart phone). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 3601 via the network 3630.

Methods as described herein can be implemented by way of machine (e.g., computer processor) executable code stored on an electronic storage location of the computer system 3601, such as, for example, on the memory 3610 or electronic storage unit 3615. The machine executable or machine readable code can be provided in the form of software. During use, the code can be executed by the processor 3605. In some cases, the code can be retrieved from the storage unit 3615 and stored on the memory 3610 for ready access by the processor 3605. In some situations, the electronic storage unit 3615 can be precluded, and machine-executable instructions are stored on memory 3610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 3601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

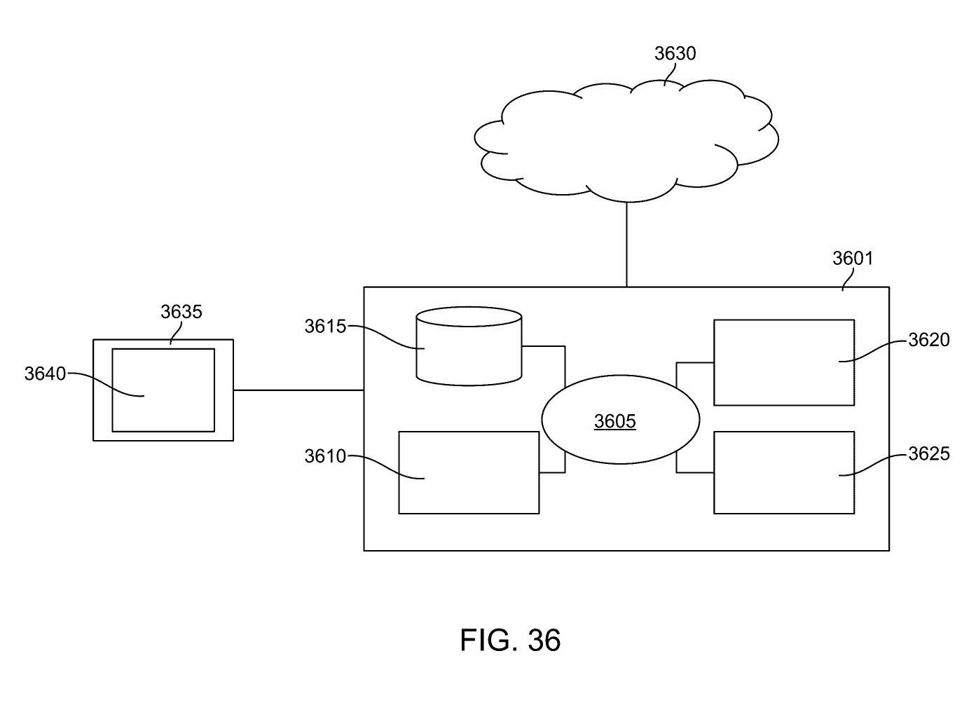

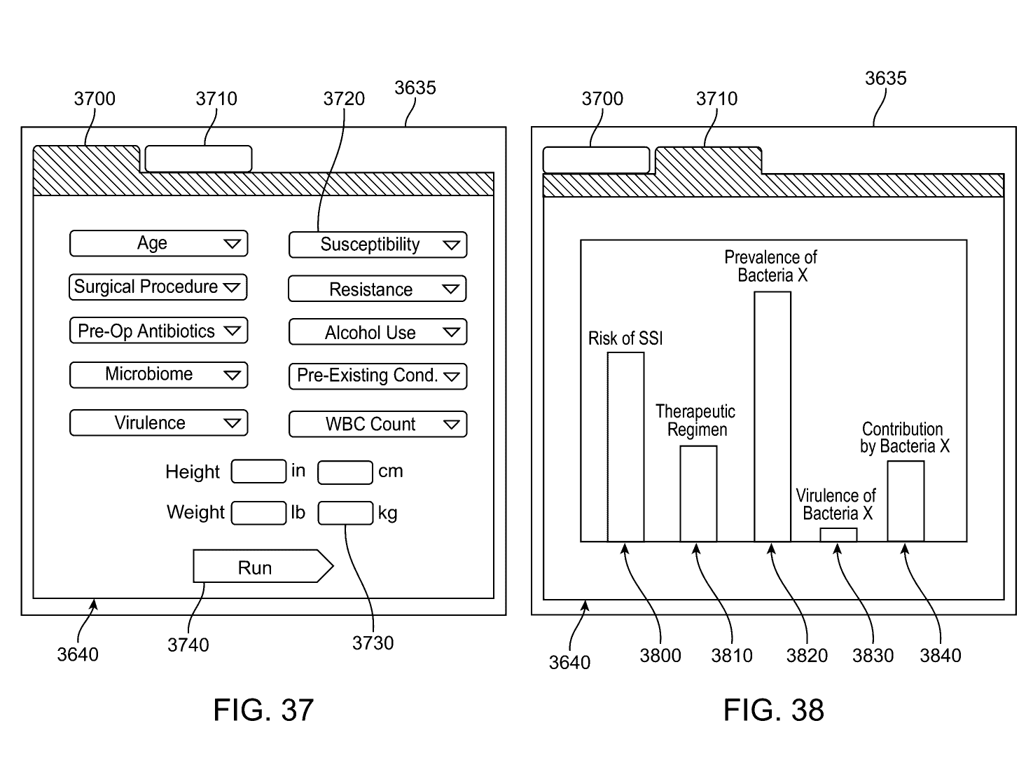

The computer system 3601 can include or be in communication with an electronic display 3635 that comprises a user interface (UI) 3640 for providing for example, patient sample input data or other input data to the neural network. Examples of UI's include, without limitation, a graphical user interface (GUI) and web-based user interface. FIGS. 37-38 show an exemplary user interface 3640 which may be provided to the user on the display 3635. Data input by a user into the user interface may be sent to the processor. The processor may be configured with instructions to run the neural network as described herein to generate one or more outputs. The output(s) of the neural network may be sent by the processor to a display which displays the outputs to a user with the user interface.

FIG. 37 shows an exemplary input user interface. The user interface 3640 may comprise a single page displaying a combination of dropdowns and text boxes which allow the user to supply various input data to the neural network and a graphical representation of the output of the nerual network. Alternatively or in combination, the input screen may be distinct from the output screen. For example, the input screen may be on an input tab 3700 and the output screen may be an output tab 3710. Alternatively of in combination, the input and output screens may be webpages. The input screen may comprise a number of dropdowns or text boxes which a user may use to deliver inputs to the neural network. For example, the input tab 3700 may comprise ten dropdowns 3720 which allow the user to input data. The inputs to the dropdowns 3720 may comprise any of the inputs or other factors described herein. The inputs may comprise the age of the patient (e.g. age or age range), the surgical procedure the patient is undergoing or underwent (e.g. surgical location, procedure type, specific procedure), whether or not the patient received pre-operative antibiotics (and when or how much), one or more components of the patient's microbiome (e.g. most prevelant species, most likely species), virulence measurements of the one or more components of the patient's microbiome (e.g. presence or absence of virulence genes or toxin production), susceptability of the one or more components of the patient's microbiome to one or more therapeutics, resistance of the one or more components of the patient's microbiome to one or more therapeutics, use of alcohol by the patient (e.g. frequency or amount), pre-existing conditions, white blood cell (WBC) counts, or any other input desired. The input tab 3700 may comprise any number of text boxes 3730, for example four as shown. Inputs to the text boxes 3730 may comprise a patient's height in inches (in) and/or in centimeters (cm) and a patient's weight in pounds (lb) or kilograms (kg). In some cases, the user may input a patient's height in either inches or centimeters and the processor may auto-populate the other text box by multiplying the input data by a conversion factor. Similarly, the user may input a patient's weight in either pounds or kilograms and the processor may auto-populate the other text box. Once the user has provide one or more input, the user may select the run button 3740 to send to the input data to the processor and input the data into the neural network as described herein. The user interface may be configured to return an error message if any of the input variables have not been provided. Alternatively, the user interface may provide the neural network with an incomplete input dataset or matrix and the neural network may be configured to instructions to provide an output with the provided subset of input variables.

FIG. 38 shows an exemplary output user interface. A user may select the output tab 3710 to switch to viewing user interface displaying the output of the neural network. The output may be displayed in the form of a graph, a list, a set of instructions or recommendations, or the like. The output data may for example be displayed as a graph. The neural network may be configured to return outputs which indicate the risk of developing surgical site infection, indicate a therapeutic regimen, identify a target microorganism likely to require therapeutics, identify risk factors which contribute to development of surgical site infection, or be any of the outputs described herein. The outputs may for example include the risk of developing a surgical site infection 3800, a recommended therapeutic regimen 3810, the prevalence of a target microorganism (“bacteria X”) 3820, the virulence of the target microorganism 3830, and the contribution of the target microorganism to the calculated risk of developing a surgical site infection 3840. The user may for example chose the output variables they would like to be displayed by the output tab 3710 or the output variables may be fixed based on the constraints of the neural network. An option to chose the output variables (not shown) may be provided on the input tab 3700, the output tab 3710, or on another user interface tab. One or both of the output tab 3710 or input tab 3700 may further be configured to enable the user to adjust input variables to determine how the outputs change in response. For example, one of the input variable may be a prophylactic therapeutic regimen and the output may be risk of surgical site infection. The user may use the user interface prior to delivering the prophylactic therapeutic in order to select a therapeutic regimen that reduces the patient's risk of surgical site infection. The user may adjust the input value upwards or downwards from a pre-determined value or from the value initially output by the neural network in order to determine how the patient's risk responds. This may be done by returning to the input page 3700 and adjusting the desired input value or by interacting directly with the outputs (e.g. by selecting the therapeutic regimen 3810 and dragging the value up or down in order to visualize the corresponding changes in the risk of surgical site infection 3800).

It will be understood by one of ordinary skill in the art that the user interface described herein may have many variations in order to provide the user with a way to input data and read an output. For example, one or more of the dropdowns 3720 may be replaced with buttons, scroll bars, steppers, radio groups, switches, sliders, text boxes, or other input mechanisms. The user interface may comprise any number or any combination of input mechanisms as desired to provide the input variables to the neural network. The output may comprise one or more graphics, one or more sets of instructions or recommendations, or the like.

Methods and systems of the present disclosure can be implemented by way of one or more algorithms. An algorithm can be implemented by way of software upon execution by the central processing unit 3605. The algorithm can, for example, comprise the neural network described herein.

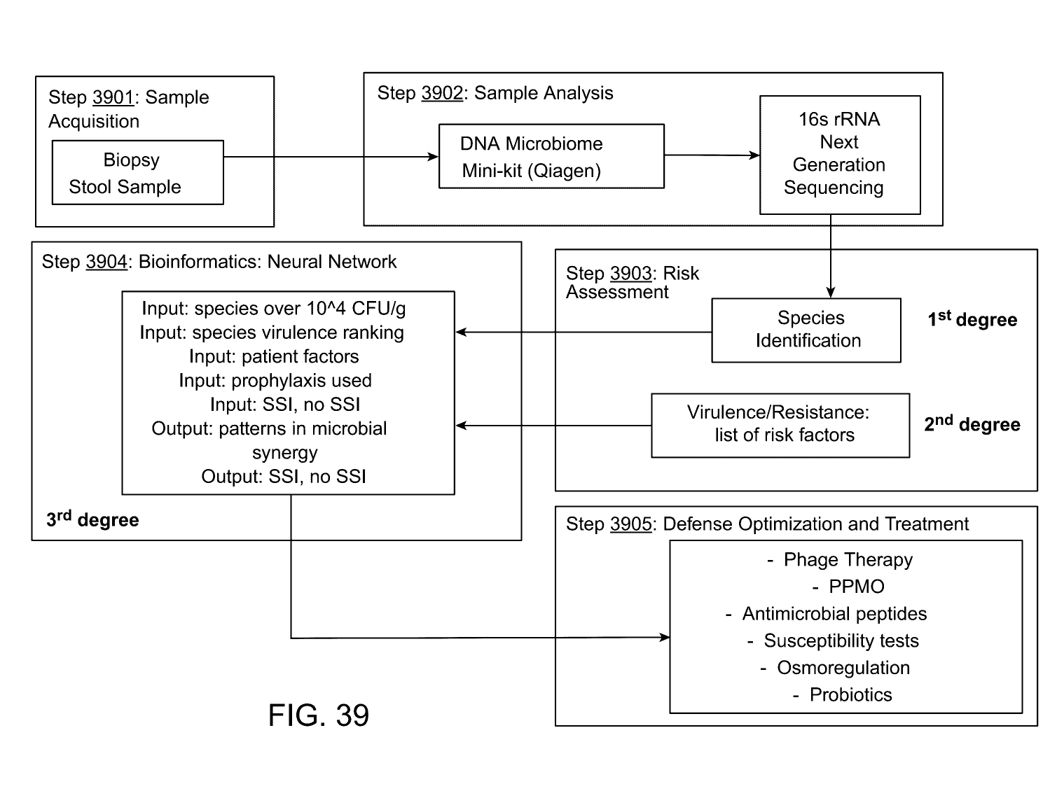

FIG. 39 shows a flowchart of a method for determining a patient's risk of developing a surgical site infection to inform prophylactic treatment.

At Step 3901, a patient sample may be acquired. The sample may comprise any of the samples described herein and may be collected using any of the methods described herein. The sample may for example comprise a stool sample and/or tissue biopsy.

At Step 3902, the sample may be analyzed. The sample may be analyzed using any of the analysis techniques described herein. For example, bacterial microbiome DNA may be purified and enriched using a commercially-available extraction kit such as the Qiagen QIAamp DNA Microbiome Kit. Extracted DNA may be sequenced using next generation 16S rRNA sequencing to classify and identify bacterial species in the sample DNA. The DNA may further be analyzed for the presence or absence of mutations or genes known to be related to virulence and/or antibody susceptibility.

At Step 3903, an optional risk assessment step may be performed on the data generated by the sample analysis. The data may for example be compared to a bacterial risk database relevant to the surgical procedure in order to identify which bacterial sample components may be most likely to cause infection as described herein. Alternatively or in combination, the data may be compared to a susceptibility database relevant to the known risk factors contributing to virulence and/or resistance of the bacterial species in the sample as described herein. The optional risk assessment step(s) may be carried out by a processor configured with instructions to compare the sample data to a bacterial risk database and/or a susceptibility database and provide the user (or one or more input nodes in the neural network directly) with a weighted dataset(s) based on the database comparisons performed. The weighted dataset may for example comprise a list of bacterial species in order of their known risk of contributing to surgical site infection.

At Step 3904, the sample data as well as other factors may be input into a neural network which may provide the user with an output as described herein. The input may for example comprise any abundant (e.g. greater than 10⁴ colony forming units/g) bacterial species found, a ranking of the virulence of the species, various patient factors, whether or not (and what) prophylaxis was used, whether or not a surgical site infection occurred in the patient, any combination thereof, or any other input described herein or known to one of ordinary skill in the art. The output may for example comprise data identifying patterns in microbial synergy, the risk (e.g. percent risk) of the patient developing a surgical site infection, a recommended therapeutic strategy, or any output (or combination of outputs) described herein or known to one of ordinary skill in the art.

At Step 3905, the output data may be used to determine a course of treatment (or treatment regimen) for the patient. The treatment may be delivered prophylactically to the patient prior to, during, or after surgery. The treatment may be delivered by any of the delivery methods described herein or known to one of ordinary skill in the art. The treatment may for example be delivered using any of the surgical devices as described herein. The course of treatment may be determined using any of the methods described herein. The course of treatment may for example be determined by consulting the hospital antibiogram as described herein. The course of treatment may be determined by the healthcare professional. Alternatively or in combination, the neural network may output a recommended course of treatment, for example based on training with a susceptibility database comprising known bacterial responses given varying virulence and resistance patterns. For example, if one of the inputs to the network is a mutation for a bacterial species known to cause resistance to a particular antibiotic, the network may output a recommendation to use an alternative therapy (or alternative dosing regimen). The treatment regimen may comprise any of the treatment strategies described herein, for example phage therapy, peptide morpholinos (PPMOs), gene therapy, anti-microbial peptides, traditional susceptibility tested antibiotics, osmoregulation, probiotics, or any combination thereof.

Although the steps above show a method of prophylactically determining the risk of a surgical site infection and treatment regimen in accordance with embodiments, a person of ordinary skill in the art will recognize many variations based on the disclosure provided herein. The steps may be completed in a different order. Steps may be added or deleted. Some of the steps may comprise sub-steps. Many of the steps may be repeated to achieve the desired therapeutic regimen.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-24. (canceled)

25. A surgical method for retracting a tissue, the method comprising:
providing a surgical device comprising a superior retention member, an inferior retention member, and a pliable membrane coupled therebetween.
inserting the inferior retention member into an wound in a body of a patient such that the superior retention member lies in a plane above the wound; and
delivering an irrigation fluid or an antibiotic to the wound using the surgical device.

26. The surgical method of claim 25, wherein delivering an antibiotic comprises delivering the antibiotic at a constant concentration.

27. The surgical method of claim 25, wherein delivering the antibiotic comprises delivering the antibiotic without the antibiotic passing through a circulatory system of the patient prior to delivery to the wound.

28. The surgical method of claim 25, wherein delivering the antibiotic comprises generating a concentration of the antibiotic in a tissue of the wound which is greater than a concentration of the antibiotic in a bloodstream of the patient.

29. The surgical method of claim 25, wherein delivering the antibiotic comprises delivering the antibiotic with minimal systemic absorption of the antibiotic.

30. The surgical method of claim 29, wherein delivering the antibiotic with minimal systemic absorption of the antibiotic reduces the risk of negative side effects to the patient.

31. The surgical method of claim 29, wherein delivering the antibiotic with minimal systemic absorption of the antibiotic reduces a risk of acquired resistance.

32. The surgical method of claim 25, wherein delivering the antibiotic provides at least a minimum inhibitory concentration of the antibiotic in a tissue of the wound.

33. The surgical method of claim 32, wherein the minimum inhibitory concentration in the tissue of the wound is reached within about 3 minutes of antibiotic delivery.

34. The surgical method of claim 32, wherein the minimum inhibitory concentration in the tissue of the wound is maintained for about 4 hours.

35. The surgical method of claim 32, wherein the minimum inhibitory concentration in the tissue of the wound is reached faster than by systemic delivery of the antibiotic.

36. The surgical method of claim 35, wherein systemic delivery comprises intravenous delivery.

37. The surgical method of claim 25, further comprising maintaining a concentration of the antibiotic in a tissue of the wound at a constant concentration while the antibiotic is being delivered to the tissue of the wound.

38. The surgical method of claim 25, further comprising maintaining a concentration of the antibiotic in a tissue of the wound at a constant concentration without using intravenous delivery.

39. The surgical method of claim 25, further comprising maintaining a concentration of the antibiotic in a tissue of the wound within a pre-determined range.

40. The surgical method of claim 25, further comprising maintaining a concentration of the antibiotic in a tissue of the wound within a pre-determined range of about 16 mg/L to about 25 mg/L.

41. The surgical method of claim 25, further comprising maintaining a concentration of the antibiotic in a tissue of the wound within a pre-determined range without using intravenous delivery.

42. The surgical method of claim 25, further comprising maintaining a concentration of the antibiotic in a tissue of the wound within about 1 mg/L of a minimum inhibitory concentration of the antibiotic to a target microorganism.

43. The surgical method of claim 25, further comprising a concentration of the antibiotic in a tissue of the wound within a pre-determined range without intervention by a user.

44. The surgical method of claim 25, further comprising maintaining a concentration of the antibiotic in a tissue of the wound within a pre-determined range independent of a surgical procedure of the patient.

45. The surgical method of claim 25, further comprising removing the fluid from the wound.

46. The surgical method of claim 45, wherein removing a fluid from the wound clears one or more microorganisms or debris from the wound.

47. The surgical method of claim 25, further comprising reducing or preventing contamination at a surface of the wound due to enteric bacteria, skin flora, gram-positive bacteria, gram-negative bacteria, aerobic bacteria, or anaerobic bacteria with the delivered antibiotic.

48. The surgical method of claim 25, further comprising neutralizing enteric bacteria, skin flora, gram-positive bacteria, gram-negative bacteria, aerobic bacteria, or anaerobic bacteria at a surface of the wound with the delivered antibiotic.

49. The surgical method of claim 25, further comprising inactivating one or more microorganisms at a surface of the wound with the delivered antibiotic.

50. The surgical method of claim 25, further comprising targeting one or more microorganisms at a surface of the wound with the delivered antibiotic.

51. The surgical method of claim 25, further comprising preventing incubation of one or more microorganisms at a surface of the wound with the delivered antibiotic.

52. The surgical method of claim 25, wherein delivering the irrigation fluid or antibiotic cleanses the wound.

53. The surgical method of claim 25, wherein delivering the irrigation fluid or antibiotic clears one or more microorganisms or debris from the wound.

54.-76. (canceled)
